(19) United States
(12) Patent Application Publication
(10) Pub. No.: US 2026/0317726 A1
(43) Pub. Date: Oct. 15, 2026

(54) METHODS AND COMPOSITIONS OF THERAPEUTIC EXTRACELLULAR VESICLES

(71) Applicant: The Florida State University Research Foundation, Inc., Tallahassee, FL (US)

(72) Inventors: Yan Li, Tallahassee, FL (US); Justice Ene, Tallahassee, FL (US)

(21) Appl. No.: 19/644,636

(22) Filed: Apr. 10, 2026

Related U.S. Application Data
(60) Provisional application No. 63/787,404, filed on Apr. 11, 2025.

(51) Int. Cl. C12N 5/071

(52) U.S. Cl. CPC C12N 5/0692; C12N 2501/115; C12N 2501/415; C12N 2506/45; C12N 2513/00; C12N 2531/00

(57) ABSTRACT

The present disclosure comprises methods and compositions for scalable production of extracellular vesicles (EVs) from human blood vessel organoids using vertical-wheel bioreactors, yielding EVs with defined lipidomic profiles and demonstrated therapeutic potential for cell-free therapies.

### CROSS REFERENCE TO RELATED APPLICATIONS

This application claims priority to U.S. Provisional Application No. 63/787,404, filed on Apr. 11, 2025, the disclosure of which is incorporated herein by reference in its entirety.

### STATEMENT REGARDING FEDERALLY SPONSORED RESEARCH

This invention was made with government support under R01 NS125016 awarded by the National Institutes of Health and NSF1917618 awarded by the National Science Foundation. The government has certain rights in the invention.

### FIELD

The present disclosure comprises methods and compositions for scalable production of extracellular vesicles (EVs) from organoids using vertical-wheel bioreactors, yielding EVs with defined lipidomic profiles and demonstrating therapeutic potential for cell-free therapies.

### BACKGROUND

Extracellular vesicles (EVs) are cell-derived, membrane-bound structures that have emerged as important participants in intercellular communication and molecular transport. EVs are known to contain a variety of biomolecules, including nucleic acids, proteins, enzymes, and lipids, and have been associated with numerous physiological and pathological processes. Their reported involvement in areas such as inflammation, immunity, cancer, and neurodegenerative disease has led to increasing interest in their composition, formation, and functional properties.

To date, substantial attention has been directed to the protein and nucleic acid contents of EVs. By comparison, the lipid content of EVs has remained less fully characterized. Accordingly, EV lipid composition is believed to have important implications for EV identity, behavior, and biological function. However, the extent to which lipid composition governs or reflects EV properties remains incompletely understood. This lack of understanding has limited the ability to fully characterize EV populations and to interpret the biological significance of lipid-associated variation among EVs.

Conventional approaches for lipid isolation, detection, and analysis present additional challenges. For example, existing techniques may be limited by one or more of sensitivity, reproducibility, throughput, standardization, accessibility, cost, or analytical resolution. These limitations are particularly relevant in the EV context, where vesicle populations may be heterogeneous and where subtle lipidomic differences may be biologically meaningful. As a result, the generation of robust, comparable, and information-rich lipidomic datasets for EVs remains difficult.

Further, the role of lipids in EV generation has not been fully resolved. Lipids are believed to participate in membrane remodeling processes associated with vesicle biogenesis, budding, release, and trafficking. However, the relationships among lipid structure, membrane properties, and EV generation remain insufficiently defined. Improved understanding in this area would be beneficial for both fundamental investigation and practical applications involving controlled or scalable EV production.

Alterations in EV lipid composition have also been associated with various pathological conditions, including cancer and neurodegenerative disorders. Such findings suggest that EV lipid profiles may have value in diagnostics, disease monitoring, and therapeutic development. Nevertheless, the art still lacks sufficiently developed analytical frameworks, standardized methodologies, and reference datasets for reliably identifying and interpreting lipidomic signatures associated with particular EV populations or disease conditions.

Thus, a need remains for improved approaches relating to lipidomics and EV generation. In particular, a need exists for approaches that enable EV generation for diagnostic, therapeutic, and bioengineering applications. There exists a need for scalable platforms for EV production at clinical-grade quality, which will potentially transform the landscape of regenerative medicine and drug delivery systems.

### SUMMARY

The present disclosure relates to a scalable, reproducible platform for the production of extracellular vesicles (EVs) derived from human blood vessel organoids (hBVOs), with particular emphasis on defining and controlling their lipidomic composition. In some examples, the platform integrates microcarrier-based and aggregate-based differentiation of hBVOs within vertical-wheel bioreactors (VWBRs), enabling dynamic culture conditions that support high-yield EV production.

In one example, disclosed herein is a method of producing extracellular vesicles (EVs), comprising: culturing human blood vessel organoids (BVOs) derived from induced pluripotent stem cells in a vertical-wheel bioreactor (VWBR) under dynamic conditions; wherein the BVOs are cultured either as aggregates or on microcarriers; and wherein the culturing is conducted under controlled shear stress conditions at a rotation speed of 40 to 80 rpm, thereby increasing EV yield compared to static culture conditions.

Some examples, further comprising isolating EVs from the culture medium and characterizing the EVs by nanoparticle tracking analysis, electron microscopy, and Western blotting to confirm exosomal characteristics.

In some examples, the BVOs are cultured on Synthemax II microcarriers.

In some examples, the EV yield is at least 2-fold higher than that obtained from static aggregate culture.

In some examples, culturing at 40 rpm results in higher EV yield and more aerobic metabolism compared to culturing at 80 rpm or static controls.

Some examples further analyze gene expression of EV biogenesis markers to confirm increased EV production activity.

In some examples, the EVs exhibit therapeutic activity in reducing oxidative stress and promoting cell proliferation in a D-galactose-induced cellular senescence model.

In one example, disclosed herein is a method of producing extracellular vesicles, comprising: culturing stem cells in a vertical-wheel bioreactor (VWBR) containing culture media to differentiate the stem cells into organoids, wherein cells within the organoids secrete extracellular vesicles (EVs) into the culture media; collecting a culture media supernatant, wherein the culture media supernatant comprises EVs secreted by the cells within the organoids; filtering the culture media supernatant to obtain the EVs, thereby obtaining filtered culture media supernatant; isolating the EVs from the filtered culture media supernatant, thereby obtaining isolated EVs; and concentrating the isolated EVs, wherein the EVs are produced in a greater amount per unit volume of culture medium than EVs produced from a corresponding static culture of organoids.

In some examples, the stem cells are human induced pluripotent stem cells.

In some examples, the organoids comprise human induced blood vessel organoids (iBVOs). In some examples, the human induced pluripotent stem cells are differentiated into organoids on microcarriers in a vertical wheel bioreactor.

In some examples, the microcarriers comprise synthetic matrix-coated microcarriers.

In some examples, the microcarriers are present at a concentration of about 15 g/L to about g/L.

In some examples, the human induced pluripotent stem cells are seeded into VWBR at about ×104 cells/mL to about 8×10⁴ cells/mL.

In some examples, the VWBR is operated at an agitation rate of about 40 rpm to about 80 rpm during differentiating stem cells.

In some examples, the stem cells are cultured in mTeSR medium for about 2 days and subsequently in DMEM/F12 medium comprising B27, Wnt activator, and fibroblast growth factor for about 10 days.

In some examples, the Wnt activator comprises CHIR99021.

In some examples, the fibroblast growth factor comprises FGF2.

In some examples, the iB VOs express one or more vascular markers selected from the group consisting of CD31, ZO-1, VE-cadherin, VEGF, and CD105.

In some examples, the culture media supernatant is collected after about 12 days.

In some examples, filtering the culture media supernatant comprises centrifuging at about g for about 5 minutes.

In some examples, isolating the filtered EVs comprise ultracentrifugation.

In some examples, concentrating the isolated EVs comprises contacting the isolated EVs with polyethylene glycol (PEG) and NaCl to precipitate the EVs.

In some examples, the EVs have a mode particle size of about 150 nm to about 250 nm.

In some examples, the EVs express one or more markers selected from the group consisting of HSC70, TSG101, and CD81.

In some examples, the EVs are produced from about 2-fold to about 6-fold greater amount per mL of culture media than EVs produced from the corresponding static culture of organoids.

In some examples, the VWBR upregulates one or more EV biogenesis genes in the cells within the organoids relative to the corresponding static culture of organoids.

In some examples, the one or more EV biogenesis genes are selected from the group consisting of Rab7a, Rab27b, SMPD2, SMPD3, ALIX, HRS, STAM1, and STAM2.

In some examples, the VWBR upregulates one or more glycolysis-associated genes in the cells within the organoids relative to the corresponding static culture of organoids.

In some examples, the one or more glycolysis-associated genes are selected from the group consisting of HK2, PDK1, LDHA, and PKM2.

In some examples, the vertical wheel bioreactor upregulates one or more autophagy-associated genes in the cells within the organoids relative to the corresponding static culture of organoids.

In some examples, the one or more autophagy-associated genes are selected from the group consisting of AMPK, ATG5, ATG16L1, BECN1, LAMP1, and TFEB.

In some examples, the EVs comprise lipids associated with sphingolipid metabolism, wherein the lipids associated with sphingolipid metabolism comprise one or more ceramide lipids, sphingomyelin lipids, hexosylceramide lipids, or sulfated hexosyl ceramide lipids.

In some examples, the EVs comprise one or more ceramide lipids selected from ceramide-non-substituted lipids (Cer-NS) and ceramide-NDS lipids (Cer-NDS).

In some examples, the EVs comprise a lipid composition comprising at least 18 upregulated lipid species or at least 14 downregulated lipid species relative to EVs produced from a corresponding static culture of organoids.

In some examples, the at least 18 upregulated lipid species comprise at least 3 ceramide lipids and the at least 14 downregulated lipid species comprise at least 1 lysophosphatidylcholine lipid or a lysophosphatidylcholine ether lipid (LPC-O).

In some examples, the lipid composition further comprises at least 1 differentially expressed sphingomyelin lipid relative to EVs produced from the corresponding static culture of organoids.

In one example, disclosed herein is a method of producing extracellular vesicles, comprising:
differentiating stem cells in a bioreactor to obtain organoids;
collecting conditioned medium from the obtained organoids; and
isolating extracellular vesicles from the conditioned medium, wherein the extracellular vesicles are produced in a greater amount per unit volume of conditioned medium than extracellular vesicles produced from a corresponding static culture of organoids.

In some examples, the stem cells are human induced pluripotent stem cells. In some examples, the human induced pluripotent stem cells are differentiated as cell aggregates in a vertical wheel bioreactor (VWBR). In some examples, the organoids comprise blood vessel organoids obtained from human induced pluripotent stem cells. In some examples, the human induced pluripotent stem cells are differentiated on microcarriers in the vertical wheel bioreactor. In some examples, the microcarriers comprise synthetic matrix-coated microcarriers. In some examples, the microcarriers are present at a concentration of about 15 g/L to about 25 g/L. In some examples, the human induced pluripotent stem cells are seeded into the VWBR at about 5×10⁴ cells/mL to about 8×10⁴ cells/mL. In some examples, the VWBR is operated at an agitation rate of about 40 rpm to about 80 rpm during the differentiating of stem cells. In some examples, the differentiating the stem cells comprises culturing the stem cells in mTeSR medium for about 2 days. In some examples, the differentiating the stem cells further comprises subsequently culturing the stem cells in DMEM/F12 medium with B27, Wnt activator, and fibroblast growth factor for about 10 days. In some examples, the Wnt activator comprises CHIR99021. In some examples, the fibroblast growth factor comprises FGF2.

In some examples, the blood vessel organoids express one or more vascular markers selected from the group consisting of CD31, ZO-1, VE-cadherin, VEGF, and CD105.

In some examples, the conditioned medium is collected after about 12 days of differentiation.

In some examples, isolating the extracellular vesicles comprises polyethylene glycol enrichment and ultracentrifugation.

In some examples, isolating the extracellular vesicles comprises centrifuging the conditioned medium at about 500 g to about 10,000 g; adding polyethylene glycol and salt to precipitate extracellular vesicles; and ultracentrifuging at greater than 100,000 g.

In some examples, extracellular vesicle isolation includes one or more centrifugation steps at about 100 g to about 20,000 g, about 200 g to about 15,000 g, about 500 g to about 10,000 g, or combinations thereof, optionally followed by ultracentrifugation at about 50,000 g to about 200,000 g, about 75,000 g to about 175,000 g, or about 100,000 g to about 150,000 g. In some examples, PEG concentration is about 1% to about 20%, about 2% to about 15%, about 4% to about 12%, about 6% to about 10%, or about 8%. Salt concentration may be from about 0.05 M to about 2.0 M, about 0.1 M to about 1.0 M, about 0.2 M to about 0.8 M, or about 0.5 M.

In some examples, the extracellular vesicles have a mode particle size of about 150 nm to about 250 nm. In some examples, extracellular vesicle mode size is about 30 nm to about 500 nm, about 50 nm to about 400 nm, about 75 nm to about 300 nm, about 100 nm to about 250 nm, about 150 nm to about 250 nm, about 175 nm to about 225 nm, or about 200 nm.

In some examples, the extracellular vesicles comprise exosomes.

In some examples, the extracellular vesicles express one or more markers selected from the group consisting of HSC70, TSG101, and CD81.

In some examples, the extracellular vesicles are depleted in calnexin.

In some examples, the extracellular vesicles are produced from about 2-fold to about 6-fold greater amount per mL of conditioned medium than extracellular vesicles produced from the corresponding static culture. In some examples, increased yield means at least about 1.1-fold, 1.2-fold, 1.5-fold, 2-fold, 2.5-fold, 3-fold, 4-fold, 5-fold, 6-fold, 8-fold, or 10-fold relative to a control. In some examples, the vertical wheel bioreactor culture upregulates one or more extracellular vesicle biogenesis genes relative to the corresponding static culture.

In some examples, the one or more extracellular vesicle biogenesis genes are selected from the group consisting of Rab7a, Rab27b, SMPD2, SMPD3, ALIX, HRS, STAM1, and STAM2.

In some examples, the vertical wheel bioreactor culture upregulates one or more glycolysis-associated genes relative to the corresponding static culture.

In some examples, the one or more glycolysis-associated genes are selected from the group consisting of HK2, PDK1, LDHA, and PKM2.

In some examples, the vertical wheel bioreactor culture upregulates one or more autophagy-associated genes relative to the corresponding static culture. In some examples, the one or more autophagy-associated genes are selected from the group consisting of AMPK, ATG5, ATG16L1, BECN1, LAMP1, and TFEB.

In one example, disclosed herein is a population of extracellular vesicles obtained from blood vessel organoids cultured as aggregates in the vertical wheel bioreactor (VWBR).

In some examples, the blood vessel organoids are cultured at about 40 rpm.

The In some examples, the extracellular vesicles are obtained from blood vessel organoids cultured on microcarriers in the vertical wheel bioreactor.

In some examples, the blood vessel organoids are cultured at about 40 rpm.

In some examples, the extracellular vesicles obtained from the VWBR have a mode particle size of about 200 nm.

In some examples, the extracellular vesicles obtained from the VWBR comprise one or more markers selected from the group consisting of HSC70, TSG101, and CD81 and are depleted in calnexin.

In some examples, the extracellular vesicles obtained from the VWBR have a lipid composition that differs from a lipid composition of extracellular vesicles produced from a corresponding static culture of organoids, wherein the extracellular vesicles obtained from the VWBR comprise differentially expressed lipids associated with one or more metabolism or signaling pathways.

In some examples, the extracellular vesicles obtained from the VWBR comprise at least 18 upregulated lipid species or at least 14 downregulated lipid species relative to extracellular vesicles produced from the corresponding static culture of organoids.

In some examples, the extracellular vesicles obtained from the VWBR comprise at least 3 upregulated ceramide lipid species relative to extracellular vesicles produced from the corresponding static culture of organoids.

In some examples, the extracellular vesicles obtained from the VWBR comprise at least 1 downregulated lysophosphatidylcholine lipid species relative to extracellular vesicles produced from the corresponding static culture of organoids.

In some examples, the extracellular vesicles obtained from the VWBR comprise at least 1 differentially expressed sphingomyelin lipid species relative to extracellular vesicles produced from the corresponding static culture of organoids.

In some examples, the lipid composition comprises altered abundance of one or more sphingomyelin species relative to extracellular vesicles produced from the corresponding static culture.

In some examples, the one or more metabolism or signaling pathways comprise sphingolipid metabolism, sphingolipid signaling, neurotrophins signaling, AGE-RAGE signaling, necroptosis, or metabolic pathways.

In one example, disclosed herein is a pharmaceutical composition comprising: an extracellular vesicle population; and a pharmaceutically acceptable carrier, wherein the extracellular vesicle population is isolated from conditioned medium collected from blood vessel organoids differentiated from human induced pluripotent stem cells in a vertical wheel bioreactor. In some examples, the pharmaceutically acceptable carrier comprises phosphate-buffered saline.

In some examples, the extracellular vesicle population has a mean particle size of about 250 nm to about 400 nm.

In some examples, the extracellular vesicle population has a mode particle size of about 180 nm to about 200 nm.

In some examples, the extracellular vesicle population comprise exosomal markers, wherein the exosomal markers comprise HSC70, TSG101, or CD81.

In some examples, the extracellular vesicle population does not comprise calnexin.

In some examples, the extracellular vesicle population is present at a concentration of about 10⁸ particles/mL to about 10⁹ particles/mL.

In some examples, the extracellular vesicle population comprises lipids associated with sphingolipid metabolism, wherein the lipids associated with sphingolipid metabolism comprise one or more ceramide lipids, sphingomyelin lipids, hexosylceramide lipids, or sulfated hexosyl ceramide lipids.

In some examples, the extracellular vesicle population comprises one or more ceramide lipids selected from ceramide-non-substituted lipids (Cer-NS) and ceramide-NDS lipids (Cer-NDS).

In some examples, the extracellular vesicle population comprises one or more sphingomyelin lipids.

In some examples, the extracellular vesicle population comprises a lipid composition comprising at least 18 upregulated lipid species or at least 14 downregulated lipid species relative to extracellular vesicles produced from a corresponding static culture of blood vessel organoids.

In some examples, the at least 18 upregulated lipid species comprise at least 3 ceramide lipids.

In some examples, the at least 14 downregulated lipid species comprise at least 1 lysophosphatidylcholine lipid or a lysophosphatidylcholine ether lipid (LPC-O).

In some examples, the lipid composition comprises at least 1 differentially expressed sphingomyelin lipid relative to extracellular vesicles produced from the corresponding static culture of blood vessel organoids.

In one example, disclosed herein is a composition comprising extracellular vesicles (EVs), wherein the EVs comprise a defined lipidomic profile with chain lengths and saturation levels indicative of sphingolipid and neurotrophin signaling pathway activity.

In some examples, the EVs contain a phospholipid bilayer and markers selected from the group consisting of CD63, CD81, and TSG101.

In some examples, the lipid composition of the EVs shows less than 0.36% variation in total lipid content across production batches.

In some examples, the lipid profile comprises chain lengths and saturation levels optimized for therapeutic delivery and vesicle stability.

In one example, disclosed herein is a pharmaceutical composition comprising the EVs and a pharmaceutically acceptable carrier for use in treating neurodegenerative diseases or vascular pathologies In one example, disclosed herein is a method of treating a disorder in a subject, comprising administering to the subject a therapeutically effective amount of a population of extracellular vesicles or a pharmaceutical composition comprising an extracellular vesicle population; and a pharmaceutically acceptable carrier, wherein the extracellular vesicle population is isolated from conditioned medium collected from blood vessel organoids differentiated from human induced pluripotent stem cells in a vertical wheel bioreactor.

In some examples, the disorder is a neurological disorder. In some examples, the neurological disorder comprises a neurodegenerative disorder, a neuroinflammatory disorder, a cerebrovascular disorder, or a brain injury. In some examples, the neurodegenerative disorder comprises Alzheimer's disease, Parkinson's disease, Huntington's disease, amyotrophic lateral sclerosis, frontotemporal dementia, Lewy body dementia, or multiple system atrophy. In some examples, the neuroinflammatory disorder comprises multiple sclerosis, encephalitis, meningitis, neuroinflammation associated with Alzheimer's disease, neuroinflammation associated with Parkinson's disease, or neuroinflammation associated with traumatic brain injury. In some examples, the cerebrovascular disorder comprises ischemic stroke, hemorrhagic stroke, cerebral small vessel disease, vascular dementia, or cerebral ischemia. In some examples, the brain injury comprises traumatic brain injury, hypoxic-ischemic brain injury, or concussion.

In some examples, administering the population of extracellular vesicles or the pharmaceutical composition reduces oxidative stress in a target cell population in the subject. In some examples, administering the population of extracellular vesicles or the pharmaceutical composition reduces reactive oxygen species in a target cell population in the subject. In some examples, administering the population of extracellular vesicles or the pharmaceutical composition increases proliferation of a senescent cell population in the subject.

In some examples, the population of extracellular vesicles or the pharmaceutical composition is administered intravenously, intra-arterially, intrathecally, intracerebroventricularly, intranasally, intramuscularly, intraperitoneally, subcutaneously, or intracranially. In some examples, the population of extracellular vesicles or the pharmaceutical composition is administered intravenously or intranasally.

In some examples, the population of extracellular vesicles or the pharmaceutical composition is administered as a single dose or as multiple doses. In some examples, the multiple doses are administered once daily, once every other day, once weekly, or once monthly. In some examples, the population of extracellular vesicles is administered at a dose of about 1×10⁶ to about 1×10¹² particles per dose. In some examples, the population of extracellular vesicles is administered at a dose of about 1×10⁸ to about 1×10¹¹ particles per dose. In some examples, the population of extracellular vesicles is administered in a volume of about 0.1 mL to about 100 mL. In some examples, the population of extracellular vesicles or the pharmaceutical composition is administered for a treatment period of at least 1 day, at least 3 days, at least 1 week, at least 2 weeks, or at least 1 month.

In some examples, the population of extracellular vesicles or the pharmaceutical composition is administered in combination with at least one additional therapeutic agent. In some examples, the at least one additional therapeutic agent comprises an anti-inflammatory agent, a neuroprotective agent, an anti-oxidant, a cerebrovascular therapeutic, or a dopaminergic agent.

In some examples, the subject is a human subject.

In one example, disclosed herein is a system for making extracellular vesicles, comprising: a vertical wheel bioreactor to culture human induced pluripotent stem cells under three-dimensional differentiation conditions to form blood vessel organoids; an agitation control to maintain an agitation rate effective to support organoid formation and extracellular vesicle production; and an extracellular vesicle isolation module to receive conditioned medium from the vertical wheel bioreactor and isolate extracellular vesicles therefrom.

In some examples, the agitation control is configured to operate the vertical wheel bioreactor at about 40 rpm to about 80 rpm. In some examples, the extracellular vesicle isolation module comprises a polyethylene glycol precipitation component and an ultracentrifugation component. In some examples, the vertical wheel bioreactor comprises a vertically rotating wheel impeller configured to generate mixed radial and axial flow.

In one example, disclosed herein is a method of producing extracellular vesicles (EVs), comprising: culturing human blood vessel organoids (BVOs) derived from induced pluripotent stem cells in a vertical-wheel bioreactor (VWBR) under dynamic conditions; wherein the BVOs are cultured either as aggregates or on microcarriers; and wherein the culturing is conducted under controlled shear stress conditions at a rotation speed of 40 to 80 rpm, thereby increasing EV yield compared to static culture conditions.

Some examples, further comprising isolating EVs from the culture medium and characterizing the EVs by nanoparticle tracking analysis, electron microscopy, and Western blotting to confirm exosomal characteristics.

In some examples, the BVOs are cultured on Synthemax II microcarriers.

In some examples, the EV yield is at least 2-fold higher than that obtained from static aggregate culture.

In some examples, culturing at 40 rpm results in higher EV yield and more aerobic metabolism compared to culturing at 80 rpm or static controls.

Some examples further comprising analyzing gene expression of EV biogenesis markers to confirm increased EV production activity.

In some examples, the EVs exhibit therapeutic activity in reducing oxidative stress and promoting cell proliferation in a D-galactose-induced cellular senescence model.

In one example, disclosed herein is a composition comprising extracellular vesicles (EVs), wherein the EVs comprise a defined lipidomic profile with chain lengths and saturation levels indicative of sphingolipid and neurotrophin signaling pathway activity.

In some examples, the EVs contain a phospholipid bilayer and markers selected from the group consisting of CD63, CD81, and TSG101.

In some examples, the lipid composition of the EVs shows less than 0.36% variation in total lipid content across production batches.

In some examples, the lipid profile comprises chain lengths and saturation levels optimized for therapeutic delivery and vesicle stability.

In one example, disclosed herein is a pharmaceutical composition comprising the EVs and a pharmaceutically acceptable carrier for use in treating neurodegenerative diseases or vascular pathologies.

In some examples, a systematic and comprehensive lipidomic analysis of hBVO-derived EVs was performed, revealing unique and reproducible lipid signatures associated with each culture modality. These defined lipid profiles are critical for ensuring batch-to-batch consistency and therapeutic efficacy, thereby addressing a key challenge in the development of EV-based therapies. In some examples, the platform disclosed herein is applied for the biomanufacturing of clinical-grade EVs suitable for use in drug screening, regenerative medicine, and cell-free therapy. The dynamic integration of bioreactor culture with advanced lipidomic profiling allows for tailoring EV composition for specific therapeutic indications, offering a novel route toward personalized medicine.

In one example, the present disclosure enables the production of EVs with controlled bioactive lipid content, which may enhance vesicle stability, targeting, and functional activity. Such EVs may be utilized in the treatment of vascular pathologies, neurodegenerative disorders, and other complex diseases requiring targeted and non-cellular therapeutic delivery.

In one example, disclosed herein is a platform supports regulatory compliance by allowing scalable, defined, and reproducible production of EVs under conditions compatible with good manufacturing practices (GMP). This positions the technology for rapid translation to clinical and pharmaceutical applications.

Overall, the present invention represents a significant advancement in the field of EV biomanufacturing by providing a robust and scalable system for producing functionally tailored, clinically relevant EVs, thereby expanding the therapeutic potential of cell-free biologics in precision medicine.

### BRIEF DESCRIPTION OF FIGURES

The accompanying figures, which are incorporated in and constitute a part of this specification, illustrate several examples described below.

FIGS. 1A-1D show a schematic of experimental design. (FIG. 1A) Human iPSC seeding conditions; (FIG. 1B) Differentiation timeline; (FIG. 1C) Cell characterizations; and (FIG. 1D) Extracellular vesicle characterizations. Ag: aggregates grown in the VWBR. MC: cells attached to Matrigel coated microcarriers in the VWBR. 6-well: aggregates grown as static culture in 6-well plates. 40:40 rpm; 80:80 rpm. Created by BioRender.

FIGS. 2A-2G show a 3D blood vessel organoid differentiation from human iPSCs on microcarriers and as aggregates in VWBRs. (FIG. 2A) Culture morphology over time. Scale bar of 4x: 500 μm. Scale bar of 10x: 200 μm. For the aggregates: (FIG. 2B) Perimeter size (μm). (FIG. 2C) Area size (μm²). (FIG. 2D) Roundness ratio. (FIG. 2E) pH kinetics. (FIG. 2F) Cell viability. (FIG. 2G) Cell number growth kinetics. (Run 2 data). * indicates p<0.05. N.S.: not significant.

FIGS. 3A-3C show an endothelial and vascular differentiation. (FIG. 3A) Immunostaining of Endothelial and Vascular differentiation markers CD31, ZO-1 and VE-Cadherin. (FIG. 3B) Flow Cytometry analysis of differentiation markers CD31 and percentage differentiated of all culture conditions compared to an unstained control. (FIG. 3C) Flow Cytometry analysis of differentiation markers ZO-1 and percentage differentiated of all culture conditions compared to an unstained control. Scale bar: 100 μm.

FIGS. 4A-4D show a metabolite analysis of human iPSC-derived blood vessel organoids in VWBRs. (FIG. 4A) Changes in K⁺, Na⁺, and Ca²⁺concentrations over time. (FIG. 4B) Changes in PO2 consumption, and PCO₂ generation. (FIG. 4C) Changes in glucose consumption, lactate generation, and the ratio of lactate generation to glucose consumption. (FIG. 4D) Changes in glutamic acid generation, NH₄⁺generation, and glucose consumption per million cells FIGS. 5A-5D show an EV biogenesis, glycolysis, and autophagy pathway analysis by RT-qPCR. Comparison of mRNA expression levels at D12. (FIG. 5A) ESCRT independent genes. (FIG. 5B) ESCRT dependent genes. (FIG. 5C) Glycolytic genes. (FIG. 5D) Autophagy genes. ESCRT: endosomal sorting complex required for transport. N=3. * indicates p<0.05.

FIGS. 6A-6F show an EV isolation and characterizations. (FIG. 6A) The representative particle size distribution measured by nanoparticle tracking analysis. (FIG. 6B) EV mean and mode sizes for run 2. (FIG. 6C) EV particle numbers normalized to media volume, (i) EVs per mL media; and to cell number, (ii) EVs per million cells for run 2. (FIG. 6D) TEM images, scale bar: 100 nm. (FIG. 6E) Western blot bands of exosomal markers. (FIG. 6F) Data combined from three runs: (i) EV size, (ii) EVs/mL media, and EVs/million cells. N=3 independent runs, * indicates p<0.05. N.S. not significant.

FIGS. 7A-7F show lipidomics analysis of lipid composition of blood vessel organoid-derived EVs. (FIG. 7A) Loop diagrams of lipid subclass composition. In the color codes that display the subclasses of lipids and their percentages of the overall composition, the three numbers correspond to 6W, Ag 40, and MC 40 respectively. (FIG. 7B) Radar of changes in lipid subclass content. (FIG. 7C) Total Lipid Molecule Content of three culture conditions. (FIG. 7D) Abundance of lipid compounds with the same number of unsaturated bonds between sample groups. (FIG. 7E) Differences between lipids with different chain length. (FIG. 7F) Hierarchical Cluster heatmap of the relative quantification of each lipid.

FIGS. 8A-8C show lipidomics analysis of lipid cargo of blood vessel organoid-derived EVs for pathway analysis. (FIG. 8A) KEEG enrichment diagram and volcano plot of differential lipids between the Ag 40 and 6 W culture conditions. (FIG. 8B) KEEG enrichment diagram and volcano plot of differential lipids between the MC 40 and 6 W culture conditions. (FIG. 8C) KEEG enrichment diagram and volcano plot of differential lipids between the MC 40 and Ag 40 culture conditions.

FIGS. 9A-9C show in vitro functional assays in an iBVO senescence model for EV therapeutic evaluation. (FIG. 9A) (i) ROS reduction relative to cells induced with D-galactose. (ii) Representative ROS images; (FIG. 9B) Relative cell proliferation compared to cells induced with D-galactose. (ii) Representative cell proliferation images; (FIG. 9C) (i) Relative mitochondrial activity compared to cells induced with D-galactose. (ii) Representative mitochondrial activity images. Scale bar: 100 μm. N=3, *: p<0.05, **: p<0.01, ***: p<0.001. N.S.: not significant.

### DETAILED DESCRIPTION

The following description of the disclosure is provided as an enabling teaching of the disclosure in its best, currently known example(s). To this end, those skilled in the relevant art will recognize and appreciate that many changes can be made to the various examples of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the examples of the invention, examples of which are illustrated in the drawings and the examples. This invention may, however, be embodied in many different forms and should not be construed as limited to the examples set forth herein.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various examples, the terms “consisting essentially of” and “consisting of” can be used in place of “comprising” and “including” to provide for more specific examples and are also disclosed. As used in this disclosure and in the appended claims, the singular forms “a”, “an”, “the”, include plural referents unless the context clearly dictates otherwise.

The following definitions are provided for the full understanding of terms used in this specification.

Ranges can be expressed herein as from “about” one particular value, and/or to “about” another particular value. When such a range is expressed, another example includes from the one particular value and/or to the other particular value. Similarly, when values are expressed as approximations, by use of the antecedent “about,” it will be understood that the particular value forms another example. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10”as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, 100% or more increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, 100%, or more decrease so long as the decrease is statistically significant.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Examples defined by each of these transition terms are within the scope of this disclosure.

As used herein, the term “treating”, “treat”, or “treatment” includes preventing, delaying, reducing the severity of, ameliorating, stabilizing, slowing progression of, suppressing, palliating, or reversing one or more symptoms, causes, or consequences of a disease or condition.

As used herein, the term preventing or prevention includes reducing risk, delaying onset, decreasing likelihood, reducing incidence, or avoiding occurrence of a disease or condition.

As used herein, the term neurological disorder includes any disease, disorder, injury, degeneration, inflammation, dysfunction, or insult affecting the central nervous system, peripheral nervous system, neurovascular unit, blood-brain barrier, cerebral vasculature, or related tissues.

As used herein, the term measuring, determining, evaluating, assessing, detecting, quantifying, profiling, or characterizing refers to the use of one or more qualitative or quantitative assays. Such assays include nanoparticle tracking analysis, electron microscopy, transmission electron microscopy, flow cytometry, immunocytochemistry, immunostaining, western blot, RT-qPCR, transcript analysis, lipidomics, metabolite analysis, chromatography, mass spectrometry, functional bioassays, oxidative stress assays, proliferation assays, mitochondrial assays, or combinations thereof.

As used herein, the term significantly, statistically significant, or significant difference refers to a difference that satisfies a predetermined statistical criterion, such as p less than 0.05, or a difference that is biologically meaningful or operationally meaningful in the relevant context.

As used herein, the term effective amount, therapeutically effective amount, sufficient amount, or amount effective to means an amount of a composition sufficient to achieve a desired biological, prophylactic, diagnostic, or therapeutic result.

An effective amount may depend on the subject, disease, route, regimen, formulation, potency, particle count, vesicle source, purity, dosing frequency, and clinical context. In some examples, dose may be expressed as particles per dose, particles per kg, protein mass per dose, protein mass per kg, lipid mass per dose, or combinations thereof.

As used herein, the term “about” refers to a value that may vary by plus or minus 1%, 2%, 5%, 10%, 15%, or 20% of the stated value, depending on context, measurement variability, biological variability, process variability, instrument variability, and the practical tolerances of the relevant field. In some examples, about means plus or minus 10%. In some examples, about means plus or minus 5%.

As used herein, where a range is provided, each intervening value, subrange, and endpoint within the range is expressly contemplated. For example, a range of 40 rpm to 80 rpm includes 40 rpm, 45 rpm, 50 rpm, 55 rpm, 60 rpm, 65 rpm, 70 rpm, 75 rpm, and 80 rpm, and any subrange thereof, including 40 rpm to 60 rpm, 45 rpm to 70 rpm, 50 rpm to 80 rpm, and 55 rpm to 75 rpm.

As used herein, numerical ranges may be expressed as from approximately x to approximately y, from x to y, between x and y, at least x, no greater than y, less than y, greater than x, or any combination thereof, and each such recitation is intended to provide written description support for equivalent and nested range formulations.

As used herein, “human induced pluripotent stem cell”, human iPSC, iPSC, or pluripotent stem cell refers to a reprogrammed human cell possessing pluripotency or substantial pluripotency and the capacity to differentiate into cells of ectodermal, mesodermal, and endodermal lineages. The term includes clinical-grade iPSCs, research-grade iPSCs, cGMP-derived iPSCs, episomally reprogrammed iPSCs, virally reprogrammed iPSCs, transgene-free iPSCs, integration-free iPSCs, clonal iPSC lines, polyclonal iPSC populations, genetically modified iPSCs, reporter iPSCs, and disease-specific iPSCs.

The terms “extracellular vesicle” or “EV” or “exosome” are used interchangeably herein and shall be understood to relate to any type of vesicle that is obtainable from a cell in any form, for instance a microvesicle (e.g. any vesicle shed from the plasma membrane of a cell), an exosome (e.g. any vesicle derived from the endo-lysosomal pathway), an apoptotic body (e.g. obtainable from apoptotic cells), a microparticle (which may be derived from e.g. platelets), an ectosome (derivable from e.g. neutrophils and monocytes in serum), prostatosome (e.g. obtainable from prostate cancer cells), or a cardiosome (e.g. derivable from cardiac cells), etc. Furthermore, the said terms shall also be understood to relate to extracellular vesicle mimics, cell and/or cell membrane-based vesicles obtained through cell extrusion, membrane extrusion, vesicle extrusion, or other techniques, etc. Essentially, the present invention may relate to any type of lipid-based structure (with vesicular morphology or with any other type of suitable morphology) that can act as a delivery or transport vehicle for pharmacological agents of interest. It will be clear to the skilled artisan that when describing medical and scientific uses and applications of the EVs, the present invention normally relates to a plurality of EVs, i.e. a population of EVs which may comprise thousands, millions, billions or even trillions of EVs. As can be seen from the experimental section below, EVs may be present in concentrations such as 105, 108, 1011, 1015, 1018, 1025, 1030 EVs (often termed “particles”) per unit of volume (for instance per ml), or any other number larger, smaller or anywhere in between. In the same vein, the term “population”, which may e.g. relate to an EV comprising a certain pharmacological agent and often a certain metabolic component, shall be understood to encompass a plurality of entities constituting such a population. In other words, individual EVs when present in a plurality constitute an EV population. Thus, naturally, the present invention pertains both to individual EVs comprising pharmacological agents and populations comprising EVs comprising pharmacological agents, as will be clear to the skilled person. The dosages of EVs when applied in vivo may naturally vary considerably depending on the disease to be treated, the administration route, the pharmacological agent cargo, etc.

The term “pharmacological agent” or “pharmacologically active agent” or “therapeutic agent” or “drug” or “pharmacological drug” or “pharmacological therapeutic” are used interchangeably herein and shall be understood to relate to any molecular agent which may be used for the treatment and/or prophylaxis and/or diagnosis of a disease and/or disorder. Pharmacological agents as per the present invention encompass a wide variety of pharmacologically or pharmaceutically active agents, including (i) organic compounds with pharmacological activity which are normally synthesized via chemical synthesis, (ii) naturally derived compounds which may for instance be obtained via purification from natural sources, (iii) nucleic acid-based compounds of various kinds, for instance oligonucleotides such as siRNA, splice-switching RNA, CRISPR guide strands, short hairpin RNA (shRNA), antisense oligonucleotides, polynucleotides such as mRNA, and in particular nucleic acid-based agents which are chemically synthesized and/or which comprise chemically modified nucleotides such as 2′-O-Me, 2′-O-Allyl, 2′-O-MOE, 2′-F, 2′-CE, 2′-EA 2′-FANA, LNA, CLNA, ENA, PNA, phosphorothioates, tricyclo-DNA, etc., (iii) peptides and polypeptides (i.e. proteins) of any kind, for instance obtained via peptides synthesis or via recombinant protein production, and (iv) essentially any type of pharmacological and/or pharmaceutically active agent which can conjugated to a suitable metabolic component. The present invention is naturally applicable also to other pharmacological agents without departing from the gist of the invention, as would be clear to a person skilled in the art.

The terms “metabolic component” or “metabolic molecule” are used interchangeably herein and shall be understood to relate to any molecule that can be utilized by a cell or a derivative of a cell (such as an EV) for essentially any purpose, be it as a structural component (such as a phospholipid or a cholesteryl moiety), an intermediary in an anabolic or catabolic pathway (such as pyruvate), a co-factor for an enzyme (such as vitamin B12), or any other type of molecule that can be used, incorporated and/or in any way metabolized or included in a cell, normally an EV source cell. Non-limiting examples as per the present invention include lipids, fatty acids, mono-, di- or polysaccharides, vitamins, sterols, ganglioside, peptides, proteins, nucleosides and nucleotide, and any combinations thereof. Non-limiting examples of metabolic components include fatty acids comprising e.g. 4-30 carbon such as stearic acid, lauric acid, myristic acid, palmitic acid, arachidic acid, and behenic acid, or any derivatives thereof, especially un-saturated fatty acid derivatives thereof. Other non-limiting examples of lipids include phospholipids, sphingolipids, glycolipids, cholesterol and other sterols, mono-, di-or tri-glycerides. Additional non-limiting examples include vitamins A, B, C, D, E, K, and all vitamers thereof (for instance B7 (biotin), B9 (folate), B12, etc.), sugars and sugar analogues, nucleotides and nucleosides and analogues thereof, amino acids and their analogues, etc.

The terms “EV protein”, “exosomal protein”, “exosomal sorting domain”, “EV sorting domain”, “EV sorting protein”, “exosomal protein”, “exosomal polypeptide”, “EV polypeptide”, etc. are used interchangeably herein and shall be understood to relate to any polypeptide that can be utilized to transport a polypeptide construct (which typically comprises, in addition to the EV protein, at least one protein of interest) to a suitable vesicular structure, i.e. to a suitable EV. More specifically, said terms shall be understood as comprising any polypeptide that enables transporting, trafficking or shuttling of a polypeptide construct to a vesicular structure, such as an exosome. Examples of such exosomal sorting domains are for instance CD9, CD53, CD63, CD81, CD31, ZO-1, VE-cadherin, VEGF, CD105, HSC70, TSG101, CD81, CD54, CD50, FLOT1, FLOT2, CD49d, CD71, CD133, CD138, CD235a, ALIX, Syntenin-1, Syntenin-2, Lamp2b, TSPAN8, TSPAN14, CD37, CD82, CD151, CD231, CD102, NOTCH1, NOTCH2, NOTCH3, NOTCH4, DLL1, DLL4, JAG1, JAG2, CD49d/ITGA4, ITGB5, ITGB6, ITGB7, CD11a, CD11b, CD11c, CD18/ITGB2, CD41, CD49b, CD49c, CD49e, CD51, CD61, CD104, Fc receptors, interleukin receptors, immunoglobulins, MHC-I or MHC-II components, CD2, CD3 epsilon, CD3 zeta, CD13, CD18, CD19, CD30, CD34, CD36,CD40, CD40L, CD44, CD45, CD45RA, CD47, CD86, CD110, CD111, CD115, CD117, CD125, CD135, CD184, CD200, CD279, CD273, CD274, CD362, COL6A1, AGRN, EGFR, GAPDH, GLUR2, GLUR3, HLA-DM, HSPG2, L1CAM, LAMB1, LAMC1, LFA-1, LGALS3BP, Mac-1 alpha, Mac-1 beta, MFGE8, SLIT2, STX3, TCRA, TCRB, TCRD, TCRG, VTI1A, VTI1B, and any combinations thereof, but numerous other polypeptides capable of transporting a polypeptide construct to an EV are comprised within the scope of the present invention. The EV proteins as per the present invention are typically of human origin and can be found in various publicly available databases such as Uniprot, RCSB, etc. The EV proteins may be fused to various other proteins and/or protein domains, to for instance enhance the surface display, increase avidity, or enable interaction with particular types of binding proteins.

The terms “source cell” or “EV source cell” or “parental cell” or “cell source” or “EV-producing cell” or any other similar terminology shall be understood to relate to any type of cell that can produce EVs under suitable conditions, for instance in suspension culture or in adherent culture or any in other type of culturing system. Source cells as per the present invention may also include cells producing exosomes in vivo. The source cells per the present invention may be select from a wide range of cells and cell lines, for instance mesenchymal stem or stromal cells or fibroblasts (obtainable from e.g. bone marrow, adipose tissue, Wharton's jelly, perinatal tissue, amniotic tissues and/or fluid, tooth buds, umbilical cord blood, skin tissue, etc.), amnion cells and more specifically amnion epithelial cells optionally expressing various early markers, myeloid suppressor cells, M2 polarized macrophages, adipocytes, endothelial cells, fibroblasts, etc. Cell lines of particular interest include human umbilical cord endothelial cells (HUVECs), human embryonic kidney (HEK) cells, endothelial cell lines such as microvascular or lymphatic endothelial cells, chondrocytes, MSCs of different origin, airway or alveolar epithelial cells, fibroblasts, endothelial cells, etc. Also, immune cells such as B cells, T cells, NK cells, macrophages, monocytes, dendritic cells (DCs) are also within the scope of the present invention, and essentially any type of cell which is capable of producing EVs is also encompassed herein. As used herein, terms like exosome, small extracellular vesicle, or exosome-enriched vesicle refers to an extracellular vesicle population enriched for vesicles having characteristics associated with endosomal biogenesis, including size, marker profile, morphology, or combinations thereof. In some examples, exosomes have a diameter of about 30 nm to about 300 nm, about 50 nm to about 250 nm, about 75 nm to about 220 nm, or about 100 nm to about 200 nm.

As used herein, “extracellular vesicle population” refers to an isolated, enriched, partially purified, purified, concentrated, diluted, formulated, fractionated, or otherwise processed collection of extracellular vesicles. The population may contain one vesicle subtype or a mixture of vesicle subtypes.

As used herein, the term organoid-derived extracellular vesicle, blood vessel organoid-derived extracellular vesicle, vascular organoid-derived extracellular vesicle, or iB VO EV refers to an extracellular vesicle released by a blood vessel organoid culture, a cell within such organoid culture, or a dissociated derivative thereof.

As used herein, “isolated extracellular vesicle” means that the extracellular vesicle has been separated, enriched, concentrated, purified, partially purified, or otherwise processed away from at least a portion of the starting conditioned medium, cells, cell debris, apoptotic bodies, proteins, nucleic acids, non-vesicular particles, or contaminants.

As used herein, “conditioned medium” refers to a culture medium that has been contacted with one or more cells, tissues, organoids, spheroids, explants, or other biological materials for a period of time sufficient to permit the release, secretion, shedding, excretion, and/or accumulation of one or more biological products into the medium. Such biological products may include, without limitation, proteins, peptides, cytokines, chemokines, growth factors, nucleic acids, lipids, metabolites, extracellular vesicles, exosomes, microvesicles, and combinations thereof. In some examples, the conditioned medium comprises DMEM/F12 and B27 recovered from differentiating cell cultures on about Day 12, optionally after media exchanges on Days 1, 3, 6, 8, and 10, wherein the conditioned medium comprises extracellular vesicles and other cell-derived components.

As used herein, the term “isolating”, “recovering”, “enriching”, “purifying”, “concentrating”, “fractionating”, or “collecting” extracellular vesicles refers to any process that increases the abundance, concentration, purity, usability, or recoverability of extracellular vesicles relative to a starting material. Such processes include centrifugation, differential centrifugation, density gradient centrifugation, ultracentrifugation, tangential flow filtration, size exclusion chromatography, affinity capture, precipitation, PEG-mediated precipitation, membrane filtration, ultrafiltration, acoustic separation, electrophoretic separation, microfluidic separation, immunoselection, and combinations thereof.

As used herein, polyethylene glycol, PEG, or PEG precipitation includes the use of one or more polyethylene glycol species having any suitable average molecular weight, including PEG-4000, PEG-6000, PEG-8000, or mixtures thereof.

As used herein, the term “culture condition” refers to any controllable parameter of a biological culture system, including cell source, seeding density, aggregate size, microcarrier type, microcarrier loading, vessel geometry, working volume, fill volume, agitation rate, impeller design, oxygenation, dissolved oxygen, pH, temperature, CO², medium composition, nutrient concentration, growth factor timing, differentiation factor timing, feeding schedule, harvest time, or combinations thereof.

As used herein, the term “vertical wheel bioreactor (VWBR)”, “vertically rotating wheel bioreactor”, or “vertical impeller bioreactor” refers to a bioreactor system configured to culture cells, cell aggregates, spheroids, organoids, microcarrier-bound cells, or other biological materials in a liquid medium, wherein the system comprises a culture vessel and a wheel, impeller, or analogous mixing element arranged in a substantially vertical orientation relative to the vessel and operable to generate controlled fluid motion within the culture medium. The vertically oriented mixing element is configured to promote suspension, circulation, mass transfer, and distribution of cells, aggregates, microcarriers, nutrients, dissolved gases, signaling factors, and waste products, while reducing localized shear stress and non-uniform mixing conditions that may adversely affect culture performance.

As used herein, “microcarrier”, “cell carrier”, “culture bead”, “attachment substrate”, “particulate substrate”, or “support particle” refers to small, solid, or porous particles suitable for supporting cell attachment, cell growth, cell differentiation, or tissue formation in a suspension culture environment. Essentially, microcarriers are tiny beads ranging in size from 100 to 300 microns. They are typically made of biocompatible materials such as polystyrene, dextran, gelatin, or solubilizing material such as cross-linked polyglycolic acid (PGA). The purpose of the microcarrier is to provide a larger surface area for cell attachment, allowing expansion capacity to be amplified several fold compared to traditional flat cultures. Instead of being cultivated on a flat surface, cells are cultured on the surface of spherical microcarriers that remain in suspension during stirring, with each microcarrier carrying hundreds of cells.

As used herein, the term “yield”, extracellular vesicle yield, production, vesicle production, vesicle output, or recovery refers to an amount of extracellular vesicles produced, secreted, isolated, recovered, or measurable from a culture, process, or formulation.

As used herein, “greater amount”, increased yield, enhanced production, elevated vesicle output, or improved recovery means any statistically significant increase relative to a control. In some examples, increased yield means at least about 1.1-fold, 1.2-fold, 1.5-fold, 2-fold, 2.5-fold, 3-fold, 4-fold, 5-fold, 6-fold, 8-fold, or 10-fold relative to a control. As used herein, the term control includes a static culture control, an alternate agitation control, an alternate microcarrier control, an alternate differentiation control, a historical control, or a reference standard. As used herein, “static culture” refers to a culture condition in which cells, aggregates, or organoids are maintained in a stationary vessel without active mechanical agitation, such as in an ultra-low attachment multiwell plate. In the present example, the static culture comprises aggregates formed in ultra-low attachment 6-well plates without microcarriers. In the present disclosure, a corresponding static culture refers to a non-bioreactor culture of blood vessel organoids maintained in ultra-low attachment 6-well plates in the absence of microcarriers and agitation.

The biomarkers of the invention can be detected in an EV using lipidomics. Lipidomics allow the identification and quantification directly from lipid extracts of more than about 95% of an EV's total lipid mass, representing about a thousand individual molecular species. The majority of lipid classes are identifiable using lipidomics include choline glycerophospholipid (GPCho), ethanolamine glycerophospholipid (GPEtn), phosphatidylinositol (GPIns), phosphatidylglycerol (GPGro), phosphatidylserine (GPSer), lysoGPCho, lysoGPEtn, phosphatidic acid (GPA), lysoGPA, sphingomyelin (SM), galactosylceramide (GalCer), glucosylceramide, sulfatide, free fatty acid, triacylglycerol (TG), acylcarnitine, cholesterol and cholesterol esters, ceramide, cardiolipin, sphingosine-1-phosphate (S1P), dihydrosphingosine-1-phosphate (DHS1P), sphingosine, and lysoSM.

As used herein, the major groups of lipids are based on chemical properties and chemical structure. Two major groups of lipids include saponifiable and nonsaponifiable lipids. Saponifiable lipids include those having long-chain fatty acids esterified to a backbone molecule such as triacylglycerols, phospholipids, glycolipids, glycerophospholipids, and sphingolipids. Nonsaponifiable lipids include lipids of the cholesterol, carbon-ring compound, and isoprene derivative classes. Additional exemplary lipid classes include choline glycerophospholipid (all subclasses), ethanolamine glycerophospholipid (all subclasses), phosphatidylinositol, phosphatidylglycerol, phosphatidylserine, lyso-glycerophospholipid, lysoethanolamine glycerophospholipid, phosphatidic acid, lyso-phosphatidic acid, sphingomyelin, galactosylceramide, glucosylceramide, gangliosides, sulfatide, free fatty acid, triacylglycerol, diacylglycerol, monoacylglycerols, acyl-CoA, acylcarnitine, cholesterol, cholesterol esters, oxysterols, prostaglandins, ceramide, cardiolipin, and sphingoid base-1-phosphate classes. Each class includes multiple lipid molecular species.

As used herein, the term “lipid composition”, “lipid profile”, “lipidomic profile”, “lipid signature”, “membrane lipid composition”, or “vesicle lipid composition” refers to the qualitative and/or quantitative presence, abundance, relative abundance, class distribution, subclass distribution, chain length distribution, chain unsaturation distribution, saturation state, acyl chain composition, headgroup composition, pathway enrichment, or combinations thereof of lipids within an extracellular vesicle population.

As used herein, the term “altered lipid composition” means a lipid composition measurably different from a control with respect to one or more lipid species, one or more lipid classes, one or more subclasses, total abundance, relative abundance, chain length, chain unsaturation, saturation, pathway association, or multivariate profile.

As used herein, the term lipid species includes any individual lipid molecular species, isoform, isomer, adduct, derivative, or annotated lipid signal.

As used herein, lipid class or subclass includes monoglycerides, diglycerides, triglycerides, free fatty acids, phosphatidylcholine, phosphatidylethanolamine, lysophosphatidylcholine, lysophosphatidylglycerol, phosphatidylglycerol, phosphatidylinositol, phosphatidylserine, phosphatidic acid, ceramides, hexosylceramides, sphingomyelins, carnitine lipids, phosphatidylmethanol, ether lipids, sulfated hexosyl ceramides, or related lipids.

As used herein, the term altered abundance includes increased abundance, decreased abundance, enrichment, depletion, overrepresentation, underrepresentation, differential expression, differential abundance, pathway-associated variance, or any combination thereof.

As used herein, the term mode size, modal particle size, characteristic size, or predominant particle size refers to a particle size measurement corresponding to the most frequent particle size in a distribution, as measured by nanoparticle tracking analysis, dynamic light scattering, tunable resistive pulse sensing, electron microscopy, or another suitable method.

As used herein, the term marker refers to a protein, nucleic acid, lipid, glycan, metabolite, structural feature, size feature, morphology feature, or functional feature associated with a cell, organoid, or extracellular vesicle.

As used herein, positive exosome marker includes HSC70, TSG101, CD81, CD63, CD9, ALIX, syntenin, flotillin, and functional equivalents thereof. Negative exosome marker includes calnexin, GM130, histones, mitochondrial proteins, endoplasmic reticulum proteins, nuclear proteins, and non-vesicular contaminants.

As used herein, depleted, negative for, substantially lacking, or reduced in relation to a marker means the marker is absent, below an assay threshold, substantially lower than a cellular lysate control, lower than a positive control, or otherwise indicative of enrichment away from a contaminating cellular compartment.

Those skilled in the art will appreciate that all of the methods of the invention of the present invention can be practiced with any combination of the features described herein, including but not limited to: subject, sample, CNS disorder, and component(s) of the lipid profile, the latter including without limitation the classes and subclasses of lipids evaluated in the profile, and the manner in which they are evaluated (e.g., methodology used and method of expressing the results [for example, as an absolute amount such as weight or moles or a relative amount such as a weight %, mole % or ratio]).

The following abbreviations are used herein:
CE=cholesterol ester
DAG=diacylglycerol
DG=diacylglycerols
dm=plasmalogen linked fatty acids
FA=free fatty acid
FC=free cholesterol
LC=total lipid class
LY=lysophosphatidylcholine
LYPC=lysophosphatidylcholine
MAG=monoacylglycerol
MUFA=mono unsaturated fatty acid
PA=phosphatidic acid
PC=phosphatidylcholine
PE=phosphatidylethanolamine
PG=phosphatidylglycerol
PI=phosphatidylinositol
PL=phospholipids
PS=phosphatidylserine
PUFA=polyunsaturated fatty acid
SAT=saturated fatty acid
SFA=saturated fatty acid
SM=sphingomyelin
SP=sphingomyelin
t=trans
TAG=triacylglycerol
TG=triacylglycerol
n3=fatty acids with n3 double bonds
n6=fatty acids with n6 double bonds
n7=fatty acids with n7 double bonds
n9=fatty acids with n9 double bonds

Phospholipids are formed from four components: a backbone to which is linked two fatty acid-derived “tails” by ester linkages and one “head” group by a phosphate ester, and an alcohol. Phospholipids with a glycerol backbone are known as glycerophospholipids or phosphoglycerides. Sphingomyelin is a phospholipid with a sphingosine backbone. Phospholipids are a major component of all biological membranes, along with glycolipids and cholesterol. The head groups of the phospholipids found in biological membranes are phosphatidylcholine (lecithin), lysophosphatidyl choline, phosphatidylethanolamine, phosphatidylserine and phosphatidylinositol, whose head group can be modified by the addition of one to three more phosphate groups. While phospholipids are the major component of biological membranes, other lipid components like sphingolipids and sterols (such as cholesterol in animal cell membranes) are also found in biological membranes.

Cardiolipin (diphosphatidyl glycerol) is an important component of the mitochondrial membrane, typically present in metabolically active cells of the heart and skeletal muscle. It has also been observed in certain bacterial membranes. It serves as an insulator and stabilizes the activity of protein complexes important to the electron transport chain.

Fatty acids are unbranched hydrocarbon chains, connected by single bonds alone (saturated fatty acids) or by both single and double bonds (unsaturated fatty acids). Examples of saturated fatty acids include but are not limited to butyric acid, lauric acid, myristic acid, pentadecanoic acid, palmitic acid, stearic acid, arachidic acid, behenic acid and lignoceric acid. Examples of unsaturated fatty acids include but are not limited to linolenic acid, docosahexaenoic acid, eicosapentaenoic acid, linoleic acid, arachidonic acid, oleic acid, and erucic acid. Particular classes of fatty acids include omega-3 fatty acids (e.g., alpha-linolenic, stearidonic, eicosatrienoic, eicosatetraenoic, eicosapentaenoic, docosapentaenoic, docosahexaenoic and tetracosahexaenoic acids), omega-6 fatty acids (e.g., linoleic, gamma-linolenic, eicosadienoic, homo-gamma-linolenic, arachidonic, docosadienoic, docosatetraenoic and 4,7,10,13,16-docosapentaenoic acids) and omega-9 fatty acids (e.g., myristoleic, palmitoleic, vaccenic, oleic, eicosenoic, mead, erucic and nervonic acids). Other fatty acids include plasmalogen-linked fatty acids including but not limited to plasmalogen 16:0, plasmalogen 18:0, plasmalogen 18:1n 7 and plasmalogen 18:1n 9. Other fatty acids include but are not limited to palmitelaidic acid, elaidic acid, 8-eicosaenoic acid and 5-eicosaenoic acid.

Essential fatty acids include the polyunsaturated fatty acids, linoleic acid and alpha-linolenic acid, which are the parent compounds of the omega-6 and omega-3 fatty acid series, respectively. They are essential in the human diet since they cannot be synthesized by the body, as the enzymes to introduce a double bond at the omega-3 and omega-6 positions are absent. The essential fatty acids are important for the immune system and in blood pressure regulation, since they are used to make compounds such as prostaglandins. The brain is also highly enriched in derivatives of linolenic and linoleic acids.

The foregoing discussion applies to free fatty acids and to fatty acid moieties found incorporated into lipid molecules in other classes (e.g., diglycerides, triglycerides and phospholipids).

Triglycerides (triacylglycerols) are the most abundant dietary lipids. They are the form in which reduced carbon is stored for energy. Each triacylglycerol has a glycerol backbone to which 3 fatty acids are esterified. Most triacylglycerols are “mixed” in that the three fatty acids differ in chain length and/or number of double bonds.

Lipid metabolites are identified herein by the lipid class and the fatty acid moiety. In the context of this invention, fatty acids are identified first by the number of carbons in the molecule (e.g., 20), the number of double bonds in the molecule (e.g., 4), and lastly the position of the double bonds (e.g., n6). To illustrate, PC20: 4n6 denotes a phosphatidylcholine molecule containing a 20 carbon fatty acid with 4 double bonds at the n6 position.

A “lipid metabolite” as used herein can refer to a single species within a lipid class (e.g., PC20: 4n6), a subset of species within a lipid class (e.g., PCn6 or PCMUFA) or the entire lipid class (e.g., Total PE).

As used herein, the term “central nervous system disorder” is to be broadly construed and includes but is not limited to psychiatric disorders and neurodegenerative disorders. CNS disorders include but are not limited to disorders of thinking and cognition such as schizophrenia and delirium; amnestic disorders; disorders of mood, such as affective disorders and anxiety disorders (including post-traumatic stress disorder, separation anxiety disorder, selective mutism, reactive attachment disorder, stereotypic movement disorder, panic disorders, agoraphobia, specific phobias, social phobia, obsessive-compulsive disorder, acute stress disorder, generalized anxiety disorder, substance-induced anxiety disorder and/or anxiety disorder not otherwise specified); disorders of social behavior; disorders of learning and memory, such as learning disorders (e.g., dyslexia); motor skills disorders; communication disorders (e.g., stuttering); pervasive developmental disorders (e.g., autistic disorder, Rett's disorder, childhood disintegrative disorder, Asperger's disorder, and/or pervasive developmental disorder not otherwise specified) and dementia. Accordingly, the term “central nervous system disorder” encompasses the disorders listed above as well as depressive disorders (including major depressive disorder, dysthmyic disorder, depressive disorder not otherwise specified, postpartum depression); seasonal affective disorder; mania; bipolar disorders (including bipolar I disorder, bipolar II disorder, cyclothymic disorder, bipolar disorder not otherwise specified); attention-deficit and disruptive behavior disorders (including attention deficit disorder with hyperactivity disorder, conduct disorder, oppositional defiant disorder and/or disruptive behavior disorder not otherwise specified); drug addiction/substance abuse (including abuse of opiates, amphetamines, alcohol, hallucinogens, cannabis, inhalants, phencyclidine, sedatives, hypnotics, anxyolytics and/or cocaine); alcohol-induced disorders; amphetamine-induced disorders; caffeine-induced disorders; cannabis-induced disorders; cocaine-induced disorders; hallucinogen-induced disorders; inhalant-induced disorders; nicotine-induced disorders; opioid-induced disorders; phencyclidine-induced disorders; sedative, hypnotic or anxyolytic-induced disorders; agitation; apathy; psychoses; irritability; disinhibition; schizophreniform disorder; schizoaffective disorder; delusional disorder; brief psychotic disorder, shared psychotic disorder; substance-induced psychotic disorder; psychotic disorder not otherwise specified; unipolar disorders, mood disorders (e.g., mood disorder with psychotic features); somatoform disorders; factitious disorders; disassociative disorders; mental retardation; feeding and eating disorders of infancy or early childhood; eating disorders such as anorexia nervosa, bulimia nervosa and/or eating disorder not otherwise specified; sleeping disorders (e.g., dyssomnias such as primary insomnia, primary hypersomnia, narcolepsy, breathing-related sleep disorder and circadian rhythm sleep disorder and/or parasomnias); impulse control disorders (e.g., kleptomania, pyromania, trichotillomania, pathological gambling and/or intermittent explosive disorder); adjustment disorders; personality disorders (e.g., paranoid personality disorder, schizoid personality disorder, schizotypal personality disorder, antisocial personality disorder, borderline personality disorder, histrionic personality disorder, narcissistic personality disorder, avoidant personality disorder, dependent personality disorder and/or obsessive-compulsive personality disorder); Tic disorders (e.g., Tourette's disorder, chronic motor or vocal tic disorder, transient tic disorder and/or tic disorder not otherwise specified); elimination disorders; and any combination of the foregoing as well as any other disorder or group of disorders described in the Diagnostic and Statistical Manual of Mental Disorders-Fourth Edition (DSM-IV; the American Psychiatric Association, Washington D.C., 1994). “Central Nervous System disorders” also include other conditions that implicate the central nervous system including but not limited to neurodegenerative disorders such as Alzheimer's disease, involuntary movement disorders such as Parkinson's disease, Huntington's disease, amyotrophic lateral sclerosis (ALS), and the like. Other central nervous system disorders include without limitation epilepsy, multiple sclerosis, neurogenic pain, psychogenic pain, and migraines. In particular examples, the neuropsychiatric disorder is schizophrenia (including any one or more subtypes thereof). In other examples, the central nervous system disorder encompasses any subset of the foregoing diseases or excludes any one or more of the foregoing conditions. In particular examples, the term “central nervous system disorder” does not encompass benign and/or malignant tumors of the central nervous system.

“Biological sample” encompasses a variety of sample types obtained from an organism that may be used in a diagnostic or monitoring assay. The term encompasses blood and other liquid samples of biological origin, solid tissue samples, such as a biopsy specimen, or tissue cultures or cells derived there from and the progeny thereof. Additionally, the term may encompass circulating tumor or other cells. The term specifically encompasses a clinical sample, and further includes cells in cell culture, cell supernatants, cell lysates, serum, plasma, urine, amniotic fluid, biological fluids, and tissue samples. The term also encompasses samples that have been manipulated in any way after procurement, such as treatment with reagents, solubilization, or enrichment for certain components

The biological sample may be a sample of a body fluid or a body tissue of the subject. For example, the biological sample may be a sample of blood, plasma, serum, saliva, bile, urine, feces or cerebrospinal fluid or samples derived from cells, tissues, or organs, such as a liver, from the subject. In many examples, it may be preferred to use blood, plasma or serum as a biological sample. A variety of techniques are available for obtaining a biological sample.

“Individual,” “subject,” “host,” and “patient,” used interchangeably herein, refer to any animal subject, such as a mammalian subject for whom diagnosis, treatment, or therapy is desired. In one preferred example, the individual, subject, host, or patient is a human. Other subjects may include, but are not limited to, cattle, horses, dogs, cats, guinea pigs, rabbits, rats, primates, woodchucks, ducks, and mice.

### Methods of Making Extracellular Vesicles

Extracellular vesicles (EVs) are lipid-bilayer non-replicating particles released from most cell types that encompass the components of their parent cells, including but not limited to proteins, adhesion molecules, cytoskeleton molecules, cytokines, lipids, nucleic acids, metabolites, and other organelles. EVs can range in size from about 20nm to larger than 10μm. The present disclosure provides systems and methods for extracting, isolating, and/or separating EVs from biological samples for further analysis.

In one example, disclosed herein is a method of producing extracellular vesicles (EVs), comprising: culturing human blood vessel organoids (BVOs) derived from induced pluripotent stem cells in a vertical-wheel bioreactor (VWBR) under dynamic conditions; wherein the BVOs are cultured either as aggregates or on microcarriers; and wherein the culturing is conducted under controlled shear stress conditions at a rotation speed of 40 to 80 rpm, thereby increasing EV yield compared to static culture conditions. some examples, the iPSCs are derived from CD34+ cells, hematopoietic cells, peripheral blood cells, peripheral blood mononuclear cells, umbilical cord blood cells, fibroblasts, epithelial cells, blood cells, somatic cells, or other suitable reprogrammable cell sources. In some examples, the iPSCs are autologous. In some examples, the iPSCs are allogeneic.

As used herein, “pluripotent cell”, “pluripotent stem cell”, “iPSC”, “induced pluripotent stem cell”, “induced pluripotent-like cell”, “embryonic stem cell”, “reprogrammed pluripotent cell”, or “functionally equivalent cell” refers to a cell capable of self-renewal and differentiation into cell types of the ectoderm, mesoderm, and endoderm.

As used herein, “three-dimensional culture”, “3D culture”, “three-dimensional cell culture”, “spatial cell culture”, or “non-planar culture” refers to a culture format in which cells are maintained in a non-planar arrangement that permits cell-cell interactions and, optionally, cell-matrix interactions in multiple dimensions. In some examples, the term includes suspension culture, aggregate culture, spheroid culture, organoid culture, scaffold-based culture, microcarrier-based culture, hydrogel culture, matrix-supported culture, or combinations thereof.

As used herein, “aggregate”, “spheroid”, “cell cluster”, “multicellular aggregate”, “three-dimensional cell aggregate”, or “multicellular assembly” refers to an assembly of cells maintained in a three-dimensional configuration. In some examples, the term includes loose aggregates, compact aggregates, organoid precursors, differentiating spheroids, vascular spheroids, and partially fused multicellular bodies.

As used herein, “organoid”, “organotypic structure”, “organ-like multicellular structure”, or “tissue-mimetic multicellular construct” refers to a three-dimensional multicellular biological structure derived from stem cells, progenitor cells, primary cells, or combinations thereof, wherein the structure recapitulates at least one structural, molecular, phenotypic, functional, developmental, or physiological characteristic of a corresponding tissue, organ, vascular network, or in vivo microenvironment.

As used herein, “blood vessel organoid”, “vascular organoid”, “vessel organoid”, “iPSC-derived blood vessel organoid”, “iBVO”, or “blood vessel-like organoid” refers to a three-dimensional cell construct comprising one or more endothelial-like, vascular-like, mural-like, tight-junction-forming, or vessel-associated cell populations and expressing one or more vascular markers. In some examples, the term includes immature blood vessel organoids, differentiating blood vessel organoids, mature blood vessel organoids, vascular spheroids, endothelial organoids, vasculogenic organoids, angiogenic organoids, and functional equivalents thereof.

In some examples, the vertical wheel bioreactor culture exhibits more aerobic metabolism than the corresponding static culture. In some examples, the more aerobic metabolism is associated with a lower lactate-to-glucose ratio than the corresponding static culture. In some examples, the vertical wheel bioreactor culture exhibits greater cell expansion than the corresponding static culture.

Some examples, further comprising isolating EVs from the culture medium and characterizing the EVs by nanoparticle tracking analysis, electron microscopy, and Western blotting to confirm exosomal characteristics.

In some examples, the BVOs are cultured on Synthemax II microcarriers.

In some examples, the EV yield is at least 2-fold higher than that obtained from static aggregate culture.

In some examples, culturing at 40 rpm results in higher EV yield and more aerobic metabolism compared to culturing at 80 rpm or static controls.

Some examples further comprise analyzing gene expression of EV biogenesis markers to confirm increased EV production activity.

In some examples, the EVs exhibit therapeutic activity in reducing oxidative stress and promoting cell proliferation in a D-galactose-induced cellular senescence model.

In one example, disclosed herein is a method of producing extracellular vesicles, comprising: differentiating stem cells in a bioreactor to obtain organoids; collecting conditioned medium from the obtained organoids; and isolating extracellular vesicles from the conditioned medium, wherein the extracellular vesicles are produced in a greater amount per unit volume of conditioned medium than extracellular vesicles produced from a corresponding static culture of organoids.

In some examples, the stem cells are human induced pluripotent stem cells. In some examples, the human induced pluripotent stem cells are differentiated as cell aggregates in a vertical wheel bioreactor (VWBR). In some examples, the organoids comprise blood vessel organoids obtained from human induced pluripotent stem cells. In some examples, a blood vessel organoid expresses one or more markers selected from CD31, PECAM1, ZO-1, TJP1, VE-cadherin, CDH5, VEGF, KDR, FLT1, CD105, ENG, Claudin family markers, Occludin, vWF, or combinations thereof. In some examples, a blood vessel organoid is characterized by endothelial morphology, barrier-associated properties, vascular-like organization, lumen-like organization, vessel sprouting, matrix deposition, or combinations thereof.

As used herein, “dynamic culture”, “dynamic bioreactor culture”, “agitated culture”, “mixed culture”, “perfused culture”, or “bioreactor culture” refers to a culture environment in which cells or organoids are subjected to motion, fluid displacement, mixing, perfusion, agitation, circulation, or other controlled hydrodynamic conditions, as compared with a static culture. In some examples, the dynamic culture is continuous. In some examples, the dynamic culture is intermittent. In some examples, the dynamic culture is orbital, rotational, perfused, stirred, rocked, wave-driven, or recirculated.

As used herein, “static culture”, “static control”, “static suspension culture”, “static aggregate culture”, or “plate-based culture” refers to a culture maintained without active bulk fluid mixing generated by a bioreactor impeller. In some examples, the term includes ultra-low attachment well plate cultures, dish cultures, flask cultures, hanging-drop cultures, non-agitated suspension cultures, and functionally similar controls.

As used herein, “corresponding static culture” refers to a control culture that shares one or more relevant features with the dynamic culture, including, but not limited to, cell source, lineage, differentiation protocol, medium formulation, culture duration, seeding density, organoid lineage, assay timing, extracellular vesicle isolation workflow, or combinations thereof, while differing in that the control culture is maintained under static rather than dynamic conditions.

As used herein, “agitation rate” refers to a rotational, mixing, or impeller speed applied to the culture environment. In some examples, agitation rate may be expressed in revolutions per minute, angular velocity, tip speed, Reynolds number, power input, energy dissipation rate, or other hydrodynamic metrics. In some examples, the agitation rate is from about 10 rpm to about 150 rpm, about 20 rpm to about 120 rpm, about 30 rpm to about 100 rpm, about 35 rpm to about 90 rpm, about 40 rpm to about 80 rpm, about 45 rpm to about 75 rpm, or any subrange therein. In particular examples, the agitation rate is about 30 rpm, 35 rpm, 40 rpm, 45 rpm, 50 rpm, 55 rpm, 60 rpm, 65 rpm, 70 rpm, 75 rpm, or 80 rpm.

As used herein, “shear stress”, “hydrodynamic stress”, “fluid shear”, or “shear condition” refers to a mechanical force associated with fluid movement acting on a cell, aggregate, organoid, microcarrier, extracellular vesicle, or surrounding medium. In some examples, shear may be average shear, local shear, peak shear, effective shear, or apparent shear. In some examples, the shear stress is low shear, moderate shear, or high shear relative to a reference system. In some examples, the shear stress is less than about 2dyn/cm2 , less than about 1dyn/cm2 , from about 0.05 dyn/cm2 to about 1dyn/cm2 , from about 0.1 dyn/cm2 to about 0.8dyn/cm2 , from about 0.15 dyn/cm2 to about 0.6dyn/cm2 , or from about 0.2 dyn/cm2 to about 0.4dyn/cm2 .

In some examples, the vertical wheel bioreactor provides radial flow, axial flow, mixed radial and axial flow, substantially homogeneous flow, substantially homogeneous shear, lower shear heterogeneity than a conventional spinner flask or stirred tank, reduced dead zones, improved suspension uniformity, reduced aggregate settling, or combinations thereof.

In some examples, the microcarrier is coated, uncoated, porous, nonporous, deformable, rigid, synthetic, natural, biodegradable, non-biodegradable, matrix-coated, peptide-coated, polymeric, glass-based, dextran-based, cellulose-based, polystyrene-based, collagen-coated, vitronectin-coated, laminin-coated, Matrigel-associated, Synthemax-coated, or otherwise surface-modified. In some examples, microcarrier concentration is from about 1 g/L to about 50 g/L, about 5 g/L to about 40 g/L, about 10 g/L to about 30 g/L, about 15 g/L to about 25 g/L, or about 20 g/L. In some examples, surface area loading is selected to provide about 50cm 2 /L to about 20,000 cm 2/L, about 500cm 2 /L to about 10,000cm 2 /L, or about 1,000cm 2 /L to about 8,000cm 2 /L.

In some examples, the human induced pluripotent stem cells are differentiated on microcarriers in the vertical wheel bioreactor. In some examples, the microcarriers comprise synthetic matrix-coated microcarriers. In some examples, the microcarriers are present at a concentration of about 15 g/L to about 25 g/L. In some examples, the human induced pluripotent stem cells are seeded into the VWBR at about 5×10⁴ cells/mL to about 8×10⁴ cells/mL. In some examples, the VWBR is operated at an agitation rate of about 40 rpm to about 80 rpm during the differentiating of stem cells. In some examples, differentiating the stem cells comprises culturing the stem cells in mTeSR medium for about 2 days. In some examples, differentiating the stem cells further comprises subsequently culturing the stem cells in DMEM/F12 medium with B27, Wnt activator, and fibroblast growth factor for about 10 days. In some examples, the Wnt activator comprises CHIR99021. The method of claim 11, wherein the fibroblast growth factor comprises FGF2.

In some examples, a Wnt activator includes CHIR99021, GSK3 inhibitors, Wnt agonists, canonical Wnt pathway activators, or combinations thereof. In some examples, a fibroblast growth factor includes FGF2, basic fibroblast growth factor, bFGF, or functionally similar fibroblast growth factor family members.

In some examples, differentiation time is from about 1 day to about 60 days, about 3 days to about 45 days, about 5 days to about 30 days, about 7 days to about 21 days, about 10 days to about 18 days, or about 12 days. In some examples, extracellular vesicles are collected during differentiation, after differentiation, during maturation, or after maturation.

As used herein, “medium”, “culture medium”, “differentiation medium”, or “growth medium” refers to a liquid formulation that supports survival, proliferation, differentiation, maturation, maintenance, or function of cells, organoids, or extracellular vesicle-producing cultures. In some examples, the medium comprises basal medium, salts, amino acids, glucose, vitamins, lipids, growth factors, serum, serum replacement, B27, N2, GlutaMAX, antibiotics, antioxidants, buffering agents, osmolytes, and combinations thereof.

In some examples, culture temperature is about 30° C. to about 40°C, about 35° C. to about 38° C., or about 37°C. In some examples, CO2 is about 2% to about 10%, about 4% to about 8%, or about 5%. In some examples, pH is about 6.8 to about 7.8, about 7.0 to about 7.6, or about 7.1 to about 7.5.

In some examples, seeding density is from about 1×10³ cells/mL to about 1×10⁷ cells/mL, about 1×10⁴ cells/mL to about 1×10⁶ cells/mL, about 2×10⁴ cells/mL to about 5×10⁵ cells/mL, about 5×10⁴ cells/mL to about 2×10⁵ cells/mL, or about 8×10⁴ cells/mL.

In some examples, yield is normalized to culture volume, conditioned medium volume, cell number, viable cell number, starting cell number, protein mass, time, bioreactor working volume, microcarrier mass, organoid number, aggregate number, culture surface area, dose unit, or combinations thereof. In some examples, extracellular vesicle yield is expressed as particles per mL, particles per million cells, particles per vessel, particles per bioreactor run, particles per day, protein mass per mL, lipid mass per mL, nucleic acid content per mL, or combinations thereof.

The method of claim 4, wherein the blood vessel organoids express one or more vascular markers selected from the group consisting of CD31, ZO-1, VE-cadherin, VEGF, and CD105.

The method of claim 1, wherein the conditioned medium is collected after about 12 days of differentiation.

The method of claim 1, wherein isolating the extracellular vesicles comprises polyethylene glycol enrichment and ultracentrifugation.

The method of claim 1, wherein isolating the extracellular vesicles comprises: centrifuging the conditioned medium at about 500 g to about 10,000 g; adding polyethylene glycol and salt to precipitate extracellular vesicles; and ultracentrifuging at greater than 100,000 g.

In some examples, extracellular vesicle isolation includes one or more centrifugation steps at about 100 g to about 20,000 g, about 200 g to about 15,000 g, about 500 g to about 10,000 g, or combinations thereof, optionally followed by ultracentrifugation at about 50,000 g to about 200,000 g, about 75,000 g to about 175,000 g, or about 100,000 g to about 150,000 g. In some examples, PEG concentration is about 1% to about 20%, about 2% to about 15%, about 4% to about 12%, about 6% to about 10%, or about 8%. Salt concentration may be from about 0.05 M to about 2.0 M, about 0.1 M to about 1.0 M, about 0.2 M to about 0.8 M, or about 0.5 M.

In some examples, the extracellular vesicles have a mode particle size of about 150 nm to about 250 nm. In some examples, extracellular vesicle mode size is about 30 nm to about 500 nm, about 50 nm to about 400 nm, about 75 nm to about 300 nm, about 100 nm to about 250 nm, about 150 nm to about 250 nm, about 175 nm to about 225 nm, or about 200 nm.

In some examples, the extracellular vesicles comprise exosomes. In some examples, the extracellular vesicles express one or more markers selected from the group consisting of HSC70, TSG101, and CD81. In some examples, the extracellular vesicles are depleted in calnexin.

In some examples, the extracellular vesicles are produced from about 2-fold to about 6-fold greater amount per mL of conditioned medium than extracellular vesicles produced from the corresponding static culture. In some examples, increased yield means at least about 1.1-fold, 1.2-fold, 1.5-fold, 2-fold, 2.5-fold, 3-fold, 4-fold, 5-fold, 6-fold, 8-fold, or 10-fold relative to a control.

In some examples, the vertical wheel bioreactor culture upregulates one or more extracellular vesicle biogenesis genes relative to the corresponding static culture.

In some examples, the one or more extracellular vesicle biogenesis genes are selected from the group consisting of Rab7a, Rab27b, SMPD2, SMPD3, ALIX, HRS, STAM1, and STAM2.

In some examples, the vertical wheel bioreactor culture upregulates one or more glycolysis-associated genes relative to the corresponding static culture.

In some examples, the one or more glycolysis-associated genes are selected from the group consisting of HK2, PDK1, LDHA, and PKM2.

In some examples, the vertical wheel bioreactor culture upregulates one or more autophagy-associated genes relative to the corresponding static culture. In some examples, the one or more autophagy-associated genes are selected from the group consisting of AMPK, ATG5, ATG16L1, BECN1, LAMP1, and TFEB.

In one example, disclosed herein is a method of manufacturing blood vessel organoids, comprising: differentiating human induced pluripotent stem cells in a vertical wheel bioreactor under three-dimensional culture conditions to form blood vessel organoids expressing one or more markers selected from the group consisting of CD31, ZO-1, VE-cadherin, VEGF, and CD105.

In some examples, the human induced pluripotent stem cells are differentiated as aggregates.

In some examples, the human induced pluripotent stem cells are differentiated on microcarriers.

In some examples, the vertical wheel bioreactor is operated at about 40 rpm to about 80 rpm.

In some examples, the blood vessel organoids are collected or cryopreserved after differentiation.

In one example, disclosed herein is a method of selecting a culture condition for production of therapeutic extracellular vesicles, comprising: culturing human induced pluripotent stem cell-derived blood vessel organoids under a plurality of vertical wheel bioreactor conditions; isolating extracellular vesicles produced under the plurality of vertical wheel bioreactor conditions; evaluating the extracellular vesicles for extracellular vesicle yield and at least one property selected from the group consisting of lipid composition, extracellular vesicle biogenesis marker expression, oxidative stress reduction activity, and proliferation-enhancing activity; and selecting a vertical wheel bioreactor condition that provides a selected extracellular vesicle yield and a selected biological activity. In some examples, the selected vertical wheel bioreactor condition comprises aggregate culture at about 40 rpm. In some examples, the selected vertical wheel bioreactor condition comprises microcarrier culture at about 40 rpm.

The methods of loading pharmacological agents into EVs described herein are highly efficient and easily scalable, and allow for the rapid production of drug-loaded EVs in quantities needed for therapeutic administration. In certain examples of the foregoing examples, when loading of EVs directly with pharmacological agents the loading may occur in 30 minutes or less, e.g. 5 minutes or less. In some examples, loading of the EVs occurs in 30 minutes, 20 minutes, 15 minutes, 10 minutes, 5 minutes, or 1 minute. In certain examples, at least 80% of the EVs in a given EV population may be loaded with the drug in question. In a preferred example, at least 90% of the EVs are loaded with the drug. In exemplary examples, at least 91%, at least 92%, at least 93%, at least 94%, at least 95%, at least 96%, at least 97%, at least 98% or more of the EVs are loaded with the drug. Naturally, when metabolically loading EV source cells then the loading of EVs may take longer, as the loading may be dependent on transport processes and/or metabolic processes inside of EV source cells.

The methods of the present invention may also comprise exposing the EV source cells to serum starvation, hypoxia, bafilomycin, or cytokines such as TNF-alpha and/or IFN-gamma, in order to influence the yield or properties of the resulting EVs. The EV production scale and timeline will be heavily dependent on the EV-producing cell or cell line and may thus be adapted accordingly by a person skilled in the art. The methods as per the present invention may further comprise an EV purification step, wherein the EVs are purified through a procedure selected from the group of techniques comprising liquid chromatography (LC), high-performance liquid chromatography (HPLC), spin filtration, tangential flow filtration, hollow fiber filtration, centrifugation, immunoprecipitation, flow field fractionation, dialysis, microfluidic-based separation, etc., or any combination thereof. Naturally, depending on whether the EVs are loaded with the drug via metabolic loading of EV source cells or loading directly of EVs, the purification of EVs may be adapted accordingly. Typically, when loading EVs directly the EV population to be loaded has already gone through at least one purification step. On the other hand, when loading EV source cells the purification step is applied to “pre-loaded” EVs released/secreted from the EV source cells. In an advantageous example, the purification of the EVs is carried out using a sequential combination of filtration (preferably ultrafiltration (UF), tangential flow filtration or hollow fiber filtration) and size exclusion liquid chromatography (LC). This combination of purification steps results in optimized purification, which in turn leads to superior therapeutic activity. Further, as compared to ultracentrifugation (UC), which is routinely employed for purifying exosomes, sequential filtration-chromatography is considerably faster and possible to scale to higher manufacturing volumes, which is a significant drawback of the current UC methodology that dominates the prior art. Another advantageous purification methodology is tangential flow filtration (TFF), which offers scalability and purity, and may be combined with others types of purification techniques such as filtration. A typical workflow for the production of small molecule-carrying EVs comprises the steps of (1) creation of a stable cell line expressing EVs (optionally having a targeting moiety displayed on their surface), (2) in an optional step purification of large quantities of such (optionally genetically engineered) EVs, (3) introduction of at least one pharmacological agent into the EVs in question, through exposing the EV population to a conjugate comprising a pharmacological agent conjugated to a metabolic component. Another typical workflow for the production of drug-loaded EVs comprises the steps of (1) creation of a stable cell line expressing EVs (optionally having a targeting moiety displayed on their surface), (2) metabolically loading of EV source cells through culturing EV source cells in the presence of a conjugate comprising a pharmacological agent conjugated to a metabolic component, (3) purification of the drug-loaded EVs produced by the EV source cells.

### Compositions Comprising Extracellular Vesicles Derived From Vertical Wheel Bioreactor

In one example, disclosed herein is a pharmaceutical composition comprising: an extracellular vesicle population; and a pharmaceutically acceptable carrier, wherein the extracellular vesicle population is isolated from conditioned medium collected from blood vessel organoids differentiated from human induced pluripotent stem cells in a vertical wheel bioreactor. As used herein, “pharmaceutical composition”, “therapeutic composition”, “formulation”, or “dosage form” refers to a composition comprising extracellular vesicles and one or more pharmaceutically acceptable carriers, excipients, diluents, stabilizers, buffers, cryoprotectants, preservatives, tonicity agents, or delivery enhancers.

As used herein, “pharmaceutically acceptable carrier” refers to a carrier, diluent, buffer, excipient, stabilizer, vehicle, or formulation component that is compatible with administration to a subject and does not unacceptably impair biological activity. In some examples, the composition is formulated in saline, buffered saline, phosphate-buffered saline, isotonic aqueous solution, lyophilized format, frozen suspension, cryoprotective medium, hydrogel, spray, infusion solution, injectable solution, slow-release formulation, nanoparticle formulation, or combinations thereof. As used herein, “cell-free therapeutic” means a therapeutic product that does not require administration of viable whole cells as the active therapeutic agent. In some examples, a cell-free therapeutic may include extracellular vesicles, exosomes, vesicle fractions, conditioned medium fractions, purified bioactive cargo, vesicle mimetics, or combinations thereof.

In some examples, the pharmaceutically acceptable carrier comprises phosphate-buffered saline.

In some examples, the extracellular vesicle population has a mean particle size of about 250 nm to about 400 nm. In some examples, the extracellular vesicle population has a mode particle size of about 180 nm to about 200 nm. In some examples, the extracellular vesicle population comprise exosomal markers, wherein the exosomal markers comprise HSC70, TSG101, or CD81.

In some examples, the extracellular vesicle population does not comprise calnexin.

In some examples, the extracellular vesicle population is present at a concentration of about 10⁸ particles/mL to about 10⁹ particles/mL. In some examples, the extracellular vesicle population comprises lipids associated with sphingolipid metabolism, wherein the lipids associated with sphingolipid metabolism comprise one or more ceramide lipids, sphingomyelin lipids, hexosylceramide lipids, or sulfated hexosyl ceramide lipids. In some examples, the extracellular vesicle population comprises one or more ceramide lipids selected from ceramide-non-substituted lipids (Cer-NS) and ceramide-NDS lipids (Cer-NDS). In some examples, the extracellular vesicle population comprises one or more sphingomyelin lipids. In some examples, the extracellular vesicle population comprises a lipid composition comprising at least 18 upregulated lipid species or at least 14 downregulated lipid species relative to extracellular vesicles produced from a corresponding static culture of blood vessel organoids. In some examples, the at least 18 upregulated lipid species comprise at least 3 ceramide lipids. In some examples, the at least 18 upregulated lipid species comprise at least 3 ceramide lipids. In some examples, the at least 18 upregulated lipid species further comprise at least 5 sphingomyelin lipids and at least 4 hexosylceramide lipids. In some examples, the at least 18 upregulated lipid species further comprise at least 2 carnitine lipids, at least 2 phosphatidylcholine lipids, at least 1 phosphatidylglycerol lipid, and at least 1 sphingolipid. In some examples, the at least 14 downregulated lipid species comprise at least 1 lysophosphatidylcholine lipid or a lysophosphatidylcholine ether lipid (LPC-O). In some examples, the lipid composition comprises at least 1 differentially expressed sphingomyelin lipid relative to extracellular vesicles produced from the corresponding static culture of blood vessel organoids.

In one example, disclosed herein is a population of extracellular vesicles obtained from blood vessel organoids cultured as aggregates in the vertical wheel bioreactor (VWBR).

In some examples, the blood vessel organoids are cultured at about 40 rpm.

The In some examples, the extracellular vesicles are obtained from blood vessel organoids cultured on microcarriers in the vertical wheel bioreactor.

In some examples, the blood vessel organoids are cultured at about 40 rpm.

In some examples, the extracellular vesicles obtained from the VWBR have a mode particle size of about 200 nm.

In some examples, the extracellular vesicles obtained from the VWBR comprise one or more markers selected from the group consisting of HSC70, TSG101, and CD81 and are depleted in calnexin.

In some examples, the extracellular vesicles obtained from the VWBR have a lipid composition that differs from a lipid composition of extracellular vesicles produced from a corresponding static culture of organoids, wherein the extracellular vesicles obtained from the VWBR comprise differentially expressed lipids associated with one or more metabolism or signaling pathways.

In some examples, the extracellular vesicles obtained from the VWBR comprise at least 18 upregulated lipid species or at least 14 downregulated lipid species relative to extracellular vesicles produced from the corresponding static culture of organoids.

In some examples, the extracellular vesicles obtained from the VWBR comprise at least 3 upregulated ceramide lipid species relative to extracellular vesicles produced from the corresponding static culture of organoids.

In some examples, the extracellular vesicles obtained from the VWBR comprise at least 1 downregulated lysophosphatidylcholine lipid species relative to extracellular vesicles produced from the corresponding static culture of organoids.

In some examples, the extracellular vesicles obtained from the VWBR comprise at least 1 differentially expressed sphingomyelin lipid species relative to extracellular vesicles produced from the corresponding static culture of organoids.

In some examples, the lipid composition comprises altered abundance of one or more sphingomyelin species relative to extracellular vesicles produced from the corresponding static culture.

In some examples, the one or more metabolism or signaling pathways comprise sphingolipid metabolism, sphingolipid signaling, neurotrophin signaling, AGE-RAGE signaling, necroptosis, or metabolic pathways.

In one example, disclosed herein is a pharmaceutical composition comprising: an extracellular vesicle population; and a pharmaceutically acceptable carrier, wherein the extracellular vesicle population is isolated from conditioned medium collected from blood vessel organoids differentiated from human induced pluripotent stem cells in a vertical wheel bioreactor. In some examples, the pharmaceutically acceptable carrier comprises phosphate-buffered saline.

In some examples, the extracellular vesicle population has a mean particle size of about 250 nm to about 400 nm.

In some examples, the extracellular vesicle population has a mode particle size of about 180 nm to about 200 nm.

In some examples, the extracellular vesicle population comprise exosomal markers, wherein the exosomal markers comprise HSC70, TSG101, or CD81.

In some examples, the extracellular vesicle population does not comprise calnexin.

In some examples, the extracellular vesicle population is present at a concentration of about 10⁸ particles/mL to about 10⁹ particles/mL.

In some examples, the extracellular vesicle population comprises lipids associated with sphingolipid metabolism, wherein the lipids associated with sphingolipid metabolism comprise one or more ceramide lipids, sphingomyelin lipids, hexosylceramide lipids, or sulfated hexosyl ceramide lipids.

In some examples, the extracellular vesicle population comprises one or more ceramide lipids selected from ceramide-non-substituted lipids (Cer-NS) and ceramide-NDS lipids (Cer-NDS).

In some examples, the extracellular vesicle population comprises one or more sphingomyelin lipids.

In some examples, the extracellular vesicle population comprises a lipid composition comprising at least 18 upregulated lipid species or at least 14 downregulated lipid species relative to extracellular vesicles produced from a corresponding static culture of blood vessel organoids.

In some examples, the at least 18 upregulated lipid species comprise at least 3 ceramide lipids.

In some examples, the at least 14 downregulated lipid species comprise at least 1 lysophosphatidylcholine lipid or a lysophosphatidylcholine ether lipid (LPC-O).

In some examples, the lipid composition comprises at least 1 differentially expressed sphingomyelin lipid relative to extracellular vesicles produced from the corresponding static culture of blood vessel organoids.

As used herein, “chain length” refers to the total number of carbons in one or more lipid acyl chains, and “unsaturation” refers to the number of double bonds in one or more lipid acyl chains.

In some examples, altered lipid composition comprises altered abundance of ceramide species, lysophosphatidylcholine species, phosphatidylethanolamine species, sphingomyelin species, hexosylceramide species, phosphatidylcholine species, phosphatidylglycerol species, phosphatidylinositol species, phosphatidylserine species, or combinations thereof. In some examples, the altered lipid composition is associated with one or more pathways selected from sphingolipid metabolism, sphingolipid signaling, AGE-RAGE signaling, necroptosis, neurotrophin signaling, ether lipid metabolism, glycine metabolism, serine metabolism, threonine metabolism, linoleic acid metabolism, and broader metabolic pathways.

In one example, disclosed herein is a composition comprising extracellular vesicles (EVs), wherein the EVs comprise a defined lipidomic profile with chain lengths and saturation levels indicative of sphingolipid and neurotrophin signaling pathway activity.

In some examples, the EVs contain a phospholipid bilayer and markers selected from the group consisting of CD63, CD81, and TSG101.

In some examples, the lipid composition of the EVs shows less than 0.36% variation in total lipid content across production batches.

In some examples, the lipid profile comprises chain lengths and saturation levels optimized for therapeutic delivery and vesicle stability.

In one example, disclosed herein is a pharmaceutical composition comprising the EVs and a pharmaceutically acceptable carrier for use in treating neurodegenerative diseases or vascular pathologies In some examples, determining the level of a lipidomic marker may be performed using a number of techniques. In some examples, determining the level of a lipidomic marker may involve using a chromatographic technique, such as liquid chromatography (LC), high performance liquid chromatography (HPLC), gas chromatography (GC), thin layer chromatography, size exclusion or affinity chromatography. In some examples, determining the level of a lipidomic marker may involve using a mass spectrometry technique, such as gas chromatography mass spectrometry (GCMS), liquid chromatography mass spectrometry (LC-MS), direct infusion mass spectrometry or Fourier transform ion-cyclotrone-resonance mass spectrometry (FT-ICR-MS), capillary electrophoresis mass spectrometry (CE-MS), high-performance liquid chromatography coupled mass spectrometry (HPLC-MS), quadrupole mass spectrometry, any sequentially coupled mass spectrometry, such as MS-MS or MS-MS-MS, inductively coupled plasma mass spectrometry (ICP-MS), pyrolysis mass spectrometry (Py-MS), ion mobility mass spectrometry or time of flight mass spectrometry (TOF). Suitable techniques are disclosed in, e.g., Nissen, Journal of Chromatography A, 703, 1995:37-57, U.S. Pat. Nos. 4,540,884 or 5,397,894.

In some examples, determining the level of a lipidomic marker may involve using one of the following techniques: nuclear magnetic resonance (NMR), magnetic resonance imaging (MRI), Fourier transform infrared analysis (FT-IR), ultraviolet (UV) spectroscopy, refraction index (RI), fluorescent detection, radiochemical detection, electrochemical detection, light scattering (LS), dispersive Raman spectroscopy or flame ionization detection (FID). In some examples, one may use fatty acyl ester analysis for determining, for example, fatty acyl composition of a particular lipoprotein fraction, such as a particular blood lipoprotein fraction. In some examples, LC-MS may be used for determining individual lipid species, such as phospholipids or sphingolipids.

In some examples, determining the level of a lipidomic marker may involve using gas chromatography with online mass spectrometry (GCMS) and/or LCMS2 (high performance liquid chromatography with online two-dimensional mass spectrometry) with suitable internal standards using software tools, such as lipid mass spectrum analysis software (LIMSA), see e.g. Haimi et al. Methods Mol. Biol. 2009, 580, 285-94, for data processing.

In some examples, determining the level of a lipidomic marker may involve a specific chemical or biological essay. The essay may utilize one or more agents that can specifically recognize the chemical structure of a lipid metabolite or are capable of specifically identifying the lipid metabolite based on its capability to react with other compounds or its capability to elicit a response in a biological read out system. For example, in some examples, an immunoassay may be used wherein an agent, such as an antibody, that is specific for the analyte in question is used to measure the abundance of the target species.

In some examples, determining the level of a lipidomic marker may involve using two or more techniques disclosed above.

In one example, disclosed herein is a pharmaceutical composition comprising EVs comprising metabolically loaded drugs. Typically, the pharmaceutical compositions as per the present invention comprise one type of therapeutic EV (i.e. a population of EVs comprising a certain desired pharmacological agent(s)) formulated with at least one pharmaceutically acceptable excipient, but more than one type of EV population may be comprised in a pharmaceutical composition, for instance in cases where a combinatorial treatment is desirable. The at least one pharmaceutically acceptable excipient may be selected from the group comprising any pharmaceutically acceptable material, composition or vehicle, for instance a solid or liquid filler, a diluent, an excipient, a carrier, a solvent or an encapsulating material, which may be involved in e.g. suspending, maintaining the activity of or carrying or transporting the EV population from one organ, or portion of the body, to another organ, or portion of the body (e.g. from the blood to any tissue and/or organ and/or body part of interest).

The present invention also relates to cosmetic and dermatological applications of EVs comprising pharmacological agents. Thus, the present invention pertains to skin care products such as creams, lotions, gels, emulsions, ointments, pastes, powders, liniments, sunscreens, shampoos, etc., comprising a suitable EV, in order to improve and/or alleviate symptoms and problems such as dry skin, wrinkles, folds, ridges, and/or skin creases. In one example, EVs (which comprise a drug of interest) are obtained from a suitable EV-producing cell source with regenerative properties (for instance a mesenchymal stromal cell) are comprised in a cosmetic cream, lotion, or gel for use in the cosmetic or therapeutic alleviation of wrinkles, lines, folds, ridges and/or skin creases.

In yet another example, the present invention relates to EVs as per the present invention for use in medicine. Naturally, when an EV comprising a pharmacological agent in accordance with the present invention is used in medicine, it is in fact normally a population of EVs that is being used. The dose of EVs administered to a patient will depend on the amount of drug that has been loaded into the EV, the disease or the symptoms to be treated or alleviated, the administration route, the pharmacological action of the drug itself, the inherent properties of the EV, as well as various other parameters of relevance.

The EVs, the EV populations and/or the pharmaceutical compositions as per the present invention may thus be used for prophylactic and/or therapeutic purposes, e.g. for use in the prophylaxis and/or treatment and/or alleviation of various diseases and disorders. A non-limiting sample of diseases wherein the EVs as per the present invention may be applied comprises Crohn's disease, ulcerative colitis, ankylosing spondylitis, rheumatoid arthritis, multiple sclerosis, systemic lupus erythematosus, sarcoidosis, idiopathic pulmonary fibrosis, psoriasis, tumor necrosis factor (TNF) receptor-associated periodic syndrome (TRAPS), deficiency of the interleukin-1 receptor antagonist (DIRA), endometriosis, autoimmune hepatitis, scleroderma, myositis, stroke, acute spinal cord injury, vasculitis, non-alcoholic steatohepatitis (NASH), non-alcoholic fatty liver disease (NAFLD), fibrosis, Guillain-Barré syndrome, acute myocardial infarction, ARDS, sepsis, meningitis, encephalitis, liver failure, kidney failure, heart failure or any acute or chronic organ failure and the associated underlying etiology, graft-vs-host disease, Duchenne muscular dystrophy and other muscular dystrophies, lysosomal storage diseases such as Gaucher disease, Fabry's disease, MPS I, II (Hunter syndrome), and III, Niemann-Pick disease, Pompe disease, etc., neurodegenerative diseases including Alzheimer's disease, Parkinson's disease, Huntington's disease and other trinucleotide repeat-related diseases, dementia, ALS, cancer-induced cachexia, anorexia, diabetes mellitus type 2, and various cancers. Virtually all types of cancer are relevant disease targets for the present invention, for instance, Acute lymphoblastic leukemia (ALL), Acute myeloid leukemia, Adrenocortical carcinoma, AIDS-related cancers, AIDS-related lymphoma, Anal cancer, Appendix cancer, Astrocytoma, cerebellar or cerebral, Basal-cell carcinoma, Bile duct cancer, Bladder cancer, Bone tumor, Brainstem glioma, Brain cancer, Brain tumor (cerebellar astrocytoma, cerebral astrocytoma/malignant glioma, ependymoma, medulloblastoma, supratentorial primitive neuroectodermal tumors, visual pathway and hypothalamic glioma), Breast cancer, Bronchial adenomas/carcinoids, Burkitt's lymphoma, Carcinoid tumor (childhood, gastrointestinal), Carcinoma of unknown primary, Central nervous system lymphoma, Cerebellar astrocytoma/Malignant glioma, Cervical cancer, Chronic lymphocytic leukemia, Chronic myelogenous leukemia, Chronic myeloproliferative disorders, Colon Cancer, Cutaneous T-cell lymphoma, Desmoplastic small round cell tumor, Endometrial cancer, Ependymoma, Esophageal cancer, Extracranial germ cell tumor, Extragonadal Germ cell tumor, Extrahepatic bile duct cancer, Eye Cancer (Intraocular melanoma, Retinoblastoma), Gallbladder cancer, Gastric (Stomach) cancer, Gastrointestinal Carcinoid Tumor, Gastrointestinal stromal tumor (GIST), Germ cell tumor (extracranial, extragonadal, or ovarian), Gestational trophoblastic tumor, Glioma (glioma of the brain stem, Cerebral Astrocytoma, Visual Pathway and Hypothalamic glioma), Gastric carcinoid, Hairy cell leukemia, Head and neck cancer, Heart cancer, Hepatocellular (liver) cancer, Hodgkin lymphoma, Hypopharyngeal cancer, Intraocular Melanoma, Islet Cell Carcinoma (Endocrine Pancreas), Kaposi sarcoma, Kidney cancer (renal cell cancer), Laryngeal Cancer, Leukemias ((acute lymphoblastic (also called acute lymphocytic leukemia), acute myeloid (also called acute myelogenous leukemia), chronic lymphocytic (also called chronic lymphocytic leukemia), chronic myelogenous (also called chronic myeloid leukemia), hairy cell leukemia)), Lip and Oral, Cavity Cancer, Liposarcoma, Liver Cancer (Primary), Lung Cancer (Non-Small Cell, Small Cell), Lymphomas, AIDS-related lymphoma, Burkitt lymphoma, cutaneous T-Cell lymphoma, Hodgkin lymphoma, Non-Hodgkin, Medulloblastoma, Merkel Cell Carcinoma, Mesothelioma, Metastatic Squamous Neck Cancer with Occult Primary, Mouth Cancer, Multiple Endocrine Neoplasia Syndrome, Multiple Myeloma/Plasma Cell Neoplasm, Mycosis Fungoides, Myelodysplastic/Myeloproliferative Diseases, Myelogenous Leukemia, Chronic Myeloid Leukemia (Acute, Chronic), Myeloma, Nasal cavity and paranasal sinus cancer, Nasopharyngeal carcinoma, Neuroblastoma, Oral Cancer, Oropharyngeal cancer, Osteosarcoma/malignant fibrous histiocytoma of bone, Ovarian cancer, Ovarian epithelial cancer (Surface epithelial-stromal tumor), Ovarian germ cell tumor, Ovarian low malignant potential tumor, Pancreatic cancer, Pancreatic islet cell cancer, Parathyroid cancer, Penile cancer, Pharyngeal cancer, Pheochromocytoma, Pineal astrocytoma, Pineal germinoma, Pineoblastoma and supratentorial primitive neuroectodermal tumors, Pituitary adenoma, Pleuropulmonary blastoma, Prostate cancer, Rectal cancer, Renal cell carcinoma (kidney cancer), Retinoblastoma, Rhabdomyosarcoma, Salivary gland cancer, Sarcoma (Ewing family of tumors sarcoma, Kaposi sarcoma, soft tissue sarcoma, uterine sarcoma), Sézary syndrome, Skin cancer (nonmelanoma, melanoma), Small intestine cancer, Squamous cell, Squamous neck cancer, Stomach cancer, Supratentorial primitive neuroectodermal tumor, Testicular cancer, Throat cancer, Thymoma and Thymic carcinoma, Thyroid cancer, Transitional cell cancer of the renal pelvis and ureter, Urethral cancer, Uterine cancer, Uterine sarcoma, Vaginal cancer, Vulvar cancer, Waldenström macroglobulinemia, and/or Wilm's tumor.

The drug-loaded EVs as per the present invention may be administered to a human or animal subject via various different administration routes, for instance auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the characteristics of the pharmacological agent and/or the EV population as such.

### Methods of Treatment

In one example, disclosed herein is a method of treating a neurological disorder in a subject in need thereof, comprising administering to the subject a therapeutically effective amount of the population of extracellular vesicles disclosed herein or the pharmaceutical composition disclosed herein. In some examples, the neurological disorder is associated with oxidative stress, cellular senescence, vascular dysfunction, neuroinflammation, or a combination thereof. In some examples, the neurological disorder comprises a neurodegenerative disease, a neuroinflammatory disease, a cerebrovascular disease, or a brain injury.

In some examples, the neurodegenerative disease comprises Alzheimer's disease, Parkinson's disease, Huntington's disease, amyotrophic lateral sclerosis, Lewy body dementia, frontotemporal dementia, or a combination thereof. In some examples, the neuroinflammatory disease comprises multiple sclerosis, encephalitis, neuroinflammation associated with Alzheimer's disease, neuroinflammation associated with Parkinson's disease, neuroinflammation associated with brain injury, or a combination thereof. In some examples, the cerebrovascular disease comprises ischemic stroke, hemorrhagic stroke, cerebral ischemia, cerebral small vessel disease, vascular dementia, blood-brain barrier dysfunction, or a combination thereof. In some examples, the brain injury comprises traumatic brain injury, hypoxic-ischemic brain injury, concussion, or a combination thereof.

As used herein, “therapeutic effect”, “biological activity”, “functional activity”, “efficacy”, or “therapeutic property” refers to any measurable beneficial effect of a composition in vitro, ex vivo, or in vivo. In some examples, such effects include reduction of oxidative stress, reduction of reactive oxygen species, increase in cell proliferation, rescue of senescence-associated phenotype, modulation of inflammatory signaling, promotion of tissue repair, improvement of vascular function, reduction of apoptosis, promotion of survival, neuroprotection, anti-senescent activity, anti-inflammatory activity, anti-apoptotic activity, or combinations thereof.

As used herein, “reducing reactive oxygen species” includes preventing an increase in reactive oxygen species, lowering a reactive oxygen species level, normalizing a reactive oxygen species level, or decreasing fluorescence or signal associated with a reactive oxygen species assay relative to a control.

As used herein, “increasing proliferation” includes increasing cell division, increasing expansion, increasing viability-associated growth, increasing CellTrace dilution, increasing DNA synthesis, increasing colony formation, or otherwise increasing a proliferation-associated metric relative to a control.

As used herein, “senescence”, “senescent state”, or “senescence-associated condition” refers to a cellular or tissue condition characterized by one or more features selected from growth arrest, oxidative stress, DNA damage responses, altered secretory phenotype, mitochondrial dysfunction, impaired proliferation, inflammatory signaling, and metabolic dysregulation.

As used herein, “senescence-inducing agent” includes D-galactose, oxidative agents, radiation, chemotherapeutic agents, inflammatory stimuli, mitochondrial toxins, genotoxic agents, metabolic stressors, replicative exhaustion, or combinations thereof. In some examples, a subject composition is effective to reduce oxidative stress, increase proliferation, reduce inflammatory signaling, improve mitochondrial status, or combinations thereof in cells exposed to D-galactose or another senescence-inducing agent.

In some examples, the neurological disorder includes neurodegenerative disease, neuroinflammatory disease, cerebrovascular disease, ischemic injury, traumatic brain injury, spinal cord injury, dementia, Alzheimer's disease, Parkinson's disease, vascular cognitive impairment, stroke, microvascular dysfunction, blood-brain barrier dysfunction, or combinations thereof.

As used herein, “administering” includes delivering by any suitable route, including intravenous, intra-arterial, intrathecal, intracerebral, intranasal, intraparenchymal, intraventricular, subcutaneous, intramuscular, intraperitoneal, local, regional, topical, ocular, catheter-based, implant-based, perfusion-based, and combinations thereof. In some examples, administration is single-dose, repeated-dose, pulsed, continuous, intermittent, localized, systemic, prophylactic, or therapeutic.

In some examples, administration of the population of extracellular vesicles reduces oxidative stress in one or more neural cells, glial cells, endothelial cells, perivascular cells, vascular cells, or a combination thereof in the subject. In some examples, administration of the population of extracellular vesicles reduces reactive oxygen species in one or more neural cells, glial cells, endothelial cells, perivascular cells, vascular cells, or a combination thereof in the subject. In some examples, administration of the population of extracellular vesicles reduces cellular senescence in one or more neural cells, glial cells, endothelial cells, perivascular cells, vascular cells, or a combination thereof in the subject. In some examples, administration of the population of extracellular vesicles increases proliferation or regenerative capacity of one or more endothelial cells, vascular cells, neural cells, glial cells, perivascular cells, or a combination thereof in the subject. In some examples, administration of the population of extracellular vesicles improves vascular integrity, endothelial function, blood-brain barrier function, or a combination thereof in the subject. In some examples, administration of the population of extracellular vesicles mitigates one or more symptoms or pathological features of the neurological disorder.

In some examples, the population of extracellular vesicles or the pharmaceutical composition is administered intravenously, intra-arterially, intranasally, intrathecally, intracerebroventricularly, intramuscularly, subcutaneously, intracranially, or a combination thereof. In some examples, the population of extracellular vesicles or the pharmaceutical composition is administered intravenously, intranasally, intrathecally, or a combination thereof.

In some examples, the population of extracellular vesicles is administered as a single dose. In some examples, the population of extracellular vesicles is administered as a plurality of doses. In some examples, the plurality of doses is administered once daily, once every two days, once weekly, biweekly, monthly, or according to a clinically appropriate dosing regimen.

In some examples, the population of extracellular vesicles is administered at a dose of about 1×10⁶ particles to about 1×10¹² particles per administration. In some examples, the population of extracellular vesicles is administered at a dose of about 1×10⁸ particles to about 1×10¹¹ particles per administration. In some examples, the therapeutically effective amount is administered for at least one week, at least two weeks, at least one month, or longer.

In some examples, the subject is a human subject. In some examples, the subject exhibits elevated oxidative stress, elevated cellular senescence, endothelial dysfunction, blood-brain barrier dysfunction, or a combination thereof.

In some examples, the population of extracellular vesicles is administered in combination with one or more additional therapeutic agents. In some examples, the one or more additional therapeutic agents comprise an anti-inflammatory agent, an anti-oxidant, a neuroprotective agent, a cerebrovascular therapeutic, a dopaminergic agent, or a combination thereof.

Disclosed herein, in some example are methods of treating a disorder in a subject by administering a therapeutically effective amount of the extracellular vesicle population disclosed herein or a pharmaceutical composition comprising the same. In some examples, the disorder is a neurological disorder. In some examples, the neurological disorder comprises a neurodegenerative disorder, a neuroinflammatory disorder, a cerebrovascular disorder, or a brain injury. In some examples, the neurodegenerative disorder comprises Alzheimer's disease, Parkinson's disease, Huntington's disease, amyotrophic lateral sclerosis, frontotemporal dementia, Lewy body dementia, multiple system atrophy, or a combination thereof. In some examples, the neuroinflammatory disorder comprises multiple sclerosis, encephalitis, meningitis, neuroinflammation associated with Alzheimer's disease, neuroinflammation associated with Parkinson's disease, neuroinflammation associated with traumatic brain injury, or a combination thereof. In some examples, the cerebrovascular disorder comprises ischemic stroke, hemorrhagic stroke, cerebral small vessel disease, vascular dementia, cerebral ischemia, or a combination thereof. In some examples, the brain injury comprises traumatic brain injury, hypoxic-ischemic brain injury, concussion, or a combination thereof.

In some examples, administration of the extracellular vesicle population or the pharmaceutical composition reduces oxidative stress in a target cell population in the subject, reduces reactive oxygen species in a target cell population in the subject, increases proliferation of a senescent cell population in the subject, or a combination thereof. In some examples, the extracellular vesicle population or the pharmaceutical composition is administered intravenously, intra-arterially, intrathecally, intracerebroventricularly, intranasally, intramuscularly, intraperitoneally, subcutaneously, intracranially, or by a combination thereof. In some examples, administration is intravenous or intranasal. In some examples, the extracellular vesicle population or the pharmaceutical composition is administered as a single dose or as multiple doses. In some examples, the multiple doses are administered once daily, once every other day, once weekly, once monthly, or according to a clinically appropriate dosing regimen. In some examples, the extracellular vesicle population is administered at a dose of about 1×10⁶ particles to about 1×10¹² particles per administration. In some examples, the population of extracellular vesicles is administered at a dose of about 1×10⁸ particles to about 1×10¹¹ particles per administration. In some examples, the extracellular vesicle population is administered in a volume of about 0.1 mL to about 100 mL. In some examples, administration is carried out for a treatment period of at least 1 day, at least 3 days, at least 1 week, at least 2 weeks, at least 1 month, or longer. In some examples, the extracellular vesicle population or the pharmaceutical composition is administered in combination with at least one additional therapeutic agent, such as an anti-inflammatory agent, a neuroprotective agent, an anti-oxidant, a cerebrovascular therapeutic, a dopaminergic agent, or a combination thereof. In some examples, the subject is a human subject.

Disclosed herein, in one example are methods for metabolically loading EVs with pharmacological agents, wherein the methods comprise culturing EV source cells in the presence of a conjugate comprising a pharmacological agent conjugated to a metabolic component. More specifically, in one example, EV source cells are exposed to a conjugate comprising a pharmacological agent and a metabolic component which via essentially any mechanism is incorporated into the source cells and naturally into the EVs obtained from the EV source cell. The conjugate typically comprises a chemical link between the metabolic component and the pharmacological agent, and this chemical linkage may optionally dissociate, break, release, or get cleaved to release the pharmacological agent. Although advantageous, release of the pharmacological agent from the metabolic component is not a requirement, as long as the pharmacological agent can exert its pharmacological effect while bound to the metabolic component. Suitable non-limiting examples of releasable chemical linkages are disulfide bridges or thioether bonds which may become undergo reduction in reductive environments, amide bonds which may be cleaved by e.g. proteases and other enzymes, biotin-streptavidin linkages which dissociate under certain in vivo conditions, etc. There are nonetheless multiple strategies available for covalent conjugation/linkage of a pharmacological agent to a metabolic component, with non-limiting examples such as an ester bond, an amide bond, a disulfide bond, a thioether bond, a biotin-streptavidin interaction, a linkage obtained through a maleimide-NHS reaction, a linkage obtained through a EDC-NHS reaction, a stapled linkage (for instance an all-hydrocarbon staple) and various other bonds.

In further examples, the EV source cells may be cultured under cell culture conditions which favor metabolic incorporation of the metabolic component. Suitable examples of such conditions would be to use a cell culture media with low concentration or essentially complete absence of e.g. vitamin B12 or biotin, naturally except for vitamin B12 or biotin conjugated to the pharmacological agent which is to be metabolically loaded into the EVs. The low concentration/absence of the metabolic component drives the metabolic uptake, processing and incorporation, thereby increasing the loading into the EV source cell and into the EVs. Alternative ways of favoring metabolic incorporation are culturing EV source cells in under low oxygen (hypoxic conditions), cytokine exposure, and/or other forms of cellular stress, for instance exposure to agents such as bafilomycin. In another alternative example, the present invention relates to methods of metabolically loading pharmacological agents into EVs by exposing EVs directly (and not EV source cells) to conjugates comprising a pharmacological agent and a metabolic component, to enable direct incorporation into the EV as such. Non-limiting examples of such methods include loading based on conjugating a pharmacological agent to e.g. lipids such as a sphingolipid, ceramide, cholesterol, a phospholipid or a fatty acid, or a ganglioside such as GM1, or a sterol and/or a peptide or a protein (such as a conventional EV protein or a peptide/protein which interacts with an EV protein and hence an EV), or any other type of suitable metabolic component. Furthermore, the use of electroporation and/or mixing of the conjugate with e.g. a transfection reagent are also within the scope of the present invention. The loading of pharmacological agents into EVs and/or into parental cells can increase when electroporation and/or transfection reagents (e.g. liposomes, cell-penetrating peptides, cationic polymers such as PEI, lipid nanoparticles, etc.) are employed. Electroporation may be carried out using voltages in the range of 20 V/cm to 1000 V/cm, often 20 V/cm to 100 V/cm. The capacitance of the electroporation step is normally between 25 μF and 250 μF, such as between 25 μF and 125 μF, although these parameters may vary extensively depending on various factors such as the EV source cells, any genetic or chemical modification of the EV, the nature of the metabolic component, the nature of the pharmacological agent, etc.

In another example, the present invention relates to an EV comprising a pharmacological agent conjugated to a metabolic component. The pharmacological agent is typically conjugated to the metabolic component via a chemical link, which may optionally be capable of releasing the pharmacological agent. The release of the pharmacological agent may advantageously result in location of free small molecule drug e.g. inside the EV or into the EV membrane. The way chemical link may be released as a result of e.g. enzymatic cleavage, dissociation, lysis, or any other type of breaking of the chemical link.

As abovementioned, the pharmacological agents as per the present invention can be obtained from essentially the entire space of pharmaceutically and/or pharmacologically and/or diagnostically relevant agents, for instance anticancer agents, cytostatic agents, tyrosine kinase inhibitors, statins, NSAIDs, antibiotics, antifungal agents, antibacterial agents, anti-inflammatory agents, anti-fibrotics, antihypertensives, aromatase or esterase inhibitors, an anticholinergics, SSRIs, BKT inhibitors, PPAR agonists, HER inhibitors, AKT inhibitors, BCR-ABL inhibitors, signal transduction inhibitors, angiogenesis inhibitors, synthase inhibitors, ALK inhibitors, BRAF inhibitors, MEK inhibitors, PI3K inhibitors, neprilysin inhibitors, beta2-agonists, CRTH2 antagonists, FXR agonists, BACE inhibitors, sphingosine-1-phosphate receptor modulators, MAPK inhibitors, Hedgehog signaling inhibitors, MDM2 antagonists, LSD1 inhibitors, lactamase inhibitors, TLR agonists, TLR antagonists, IDO inhibitors, ERK inhibitors, Chk1 inhibitors, splicing modulatory, DNA or RNA intercalators, etc. Other non-limiting examples of pharmacological agents as per the present invention includes for instance everolimus, trabectedin, abraxane, pazopanib, enzastaurin, vandetanib, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, pemetrexed, erlotinib, dasatanib, nilotinib, decatanib, panitumumab, amrubicin, oregovomab, nolatrexed, batabulin, ofatumumab, zanolimumab, edotecarin, tetrandrine, rubitecan, tesmilifene, oblimersen, ticilimumab, ipilimumab, gossypol, cilengitide, gimatecan, lucanthone, neuradiab, vitespan, talampanel, atrasentan, romidepsin, sunitinib, 5-fluorouracil, vorinostat, etoposide, gemcitabine, doxorubicin, 5′-deoxy-5-fluorouridine, vincristine, temozolomide, seliciclib, capecitabine, camptothecin, PEG-labeled irinotecan, tamoxifen, toremifene citrate, anastrazole, exemestane, letrozole, vatalanib, goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, erlotinib, lapatanib, canertinib, lonafarnib, tipifarnib, amifostine, suberoyl analide hydroxamic acid, valproic acid, trichostatin sorafenib, arnsacrine, anagrelide, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, squalamine, endostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, droloxifene, 4-hydroxytamoxifen, pipendoxifene, arzoxifene, fulvestrant, acolbifene, lasofoxifene, idoxifene, topotecan, rapamycin, temsirolimus, zolendronate, prednisone, lenalidomide, gemtuzumab, hydrocortisone, dexrazoxane, alemtuzumab, all-transretinoic acid, ketoconazole, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa, efavirinz among others. Furthermore, as abovementioned, the pharmacological agents as per the present invention also includes naturally derived compounds which may for instance be obtained via purification from natural sources, any type of nucleic acid-based compounds, for instance oligonucleotides such as siRNA, splice-switching RNA, CRISPR guide strands, short hairpin RNA, antisense oligonucleotides, mRNA, and in particular nucleic acid-based agents which are chemically synthesized and/or which comprise chemically modified nucleotides such as 2′-O-Me, 2′-O-Allyl, 2′-O-MOE, 2′-F, 2′-CE, 2′-EA 2′-FANA, LNA, CLNA, ENA, PNA, phosphorothioates, tricyclo-DNA, etc. Furthermore, peptides and polypeptides, and not only peptides and/or proteins obtainable via peptide synthesis but also peptides and proteins obtainable through recombinant protein production, are also comprised within the definition of the pharmacological agents as per the present invention. As mentioned above, the present invention is naturally applicable also to other pharmacological agents without departing from the gist of the invention, as would be clear to a person skilled in the art.

In yet another example, the EVs as per the present invention may comprise at least one targeting moiety displayed on the surface of the EV, to even further enhance its therapeutic potential by targeting a tissue, an organ, or cell type of interest. The targeting moiety normally comprises a sequence of amino acids, which may be identified for instance through phage display or any other type of screening methodology. The targeting moiety is typically displayed on the EV surface through genetic engineering of the EV source cells, wherein the source cells are transfected to produce EVs comprising a fusion protein comprising the targeting moiety and an exosome protein. Antibodies, such as monoclonal antibodies, can also function as targeting agents, especially when attached to EV surfaces.

In a further example, the present invention relates to a method of delivering a pharmacological agent to a target cell. Such delivery methods may comprise exposing a target cell, or a target tissue or target organ (which may include fluids and liquids such as blood, interstitial fluid, cerebrospinal fluid, etc.), to an EV as per the present invention. As above-mentioned, the EVs may comprise a targeting moiety expressed on its surface, or it may rely on natural tropism and targeting, or it may be non-targeted. Delivery to a target cell can be carried out in vitro and/or in vivo, depending on the context. Further, the present invention pertains to a method of altering the pharmacokinetic or pharmacodynamics profile of a small molecule drug. This can be achieved through loading the pharmacological agent in question into an EV, which will naturally affect factors such as distribution, enzymatic activity, tissue penetration, etc.

In a further example, the present invention relates to EVs comprising a pharmacological drug, wherein the pharmacological agent is released in the EV from a conjugate comprising the pharmacological agent and a metabolic component. Depending on the nature of the chemical link, a residue (such as a thiol, a biotin, a hydroxyl group, etc.) may remain on the small molecule agent after release from the metabolic component.

### Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the extracellular vesicles, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, or topically. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and Mckenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A.R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

The pharmaceutical composition may be administered in a number of ways depending on whether local or systemic treatment is desired, and on the area to be treated. Administration may be orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid-or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

A number of examples of the disclosure have been described. Nevertheless, it will be understood that various modifications may be made without departing from the spirit and scope of the invention. Accordingly, other examples are within the scope of the following claims.

By way of non-limiting illustration, examples of certain examples of the present disclosure are given below.

### Systems and Platforms for Manufacturing EVs

In one example, disclosed herein is a system for making extracellular vesicles, comprising: a vertical wheel bioreactor to culture human induced pluripotent stem cells under three-dimensional differentiation conditions to form blood vessel organoids; an agitation control to maintain an agitation rate effective to support organoid formation and extracellular vesicle production; and an extracellular vesicle isolation module to receive conditioned medium from the vertical wheel bioreactor and isolate extracellular vesicles therefrom.

In some examples, the agitation control is configured to operate the vertical wheel bioreactor at about 40 rpm to about 80 rpm. In some examples, the extracellular vesicle isolation module comprises a polyethylene glycol precipitation component and an ultracentrifugation component. In some examples, the vertical wheel bioreactor comprises a vertically rotating wheel impeller configured to generate mixed radial and axial flow.

In some examples, a systematic and comprehensive lipidomic analysis of hBVO-derived EVs was performed, revealing unique and reproducible lipid signatures associated with each culture modality. These defined lipid profiles are critical for ensuring batch-to-batch consistency and therapeutic efficacy, thereby addressing a key challenge in the development of EV-based therapies. In some examples, the platform disclosed herein is applied for the biomanufacturing of clinical-grade EVs suitable for use in drug screening, regenerative medicine, and cell-free therapy. The dynamic integration of bioreactor culture with advanced lipidomic profiling allows for tailoring EV composition for specific therapeutic indications, offering a novel route toward personalized medicine.

In one example, the present disclosure enables the production of EVs with controlled bioactive lipid content, which may enhance vesicle stability, targeting, and functional activity. Such EVs may be utilized in the treatment of vascular pathologies, neurodegenerative disorders, and other complex diseases requiring targeted and non-cellular therapeutic delivery.

In one example, disclosed herein is a platform supports regulatory compliance by allowing scalable, defined, and reproducible production of EVs under conditions compatible with good manufacturing practices (GMP). This positions the technology for rapid translation to clinical and pharmaceutical applications.

Overall, the present invention represents a significant advancement in the field of EV biomanufacturing by providing a robust and scalable system for producing functionally tailored, clinically relevant EVs, thereby expanding the therapeutic potential of cell-free biologics in precision medicine.

### EXAMPLES

The following examples are set forth below to illustrate the compositions, devices, methods, and results according to the disclosed subject matter. These examples are not intended to be inclusive of all examples of the subject matter disclosed herein, but rather to illustrate representative methods and results. These examples are not intended to exclude equivalents and variations of the present invention which are apparent to one skilled in the art.

### Example 1: Vertical Wheel Bioreactors (VWBRs) Induced Extracellular Vesicles From Human Induced Pluripotent Stem Cells (iPSCs)

iPSCs have revolutionized biomedical research, emerging as a versatile platform for drug discovery, disease modeling, and therapeutic evaluation. With their capacity to differentiate into virtually any cell type, iPSCs have been directed in vivo to generate all major brain cell types, including neurons, neural progenitor cells, microglia, oligodendrocytes, pericytes, and vascular endothelial cells. To further bridge the gap between in vitro studies and the complexity of human biology, three-dimensional (3D) organoids derived from iPSCs have become indispensable. These 3D structures partially recapitulate and mimic the intricate cellular architecture and microenvironment in vivo, surpassing the limitations of traditional 2D cultures. By capturing the dynamic interactions and genetic intricacies of the tissues, iPSC-derived organoids have uncovered groundbreaking insights into the molecular and genetic underpinnings of complex neurological disorders, opening new avenues for understanding and treating these conditions.

Organoid generation is commonly achieved using ultra-low attachment plates, where aggregation occurs due to enhanced cell-cell adhesion in suspension, usually under static conditions. However, static cultures face significant challenges, primarily due to limitations in nutrient diffusion, which make them less ideal to mimic in vivo organ growth. The absence of microcirculation in static cultures leads to insufficient nutrient and oxygen delivery, as well as the accumulation of metabolic waste. To address these limitations, dynamic 3D culture systems utilizing bioreactors have gained increasing attention. Among the most widely used bioreactors are rotating wall and stirred tank (spinner flask) reactors. However, these systems have inherent limitations. Spinner flasks, for example, induce high shear stress due to horizontal rotation, which can create regions of varying shear rates within the reactor, leading to variability in culture conditions. Additionally, a rotating wall bioreactor has been shown to require high speeds for sufficient agitation which can result in high shear rates, and the scalability is limited.

Vertical Wheel Bioreactors (VWBRs) have been recently established for stem cell culture that addresses the limitations of commonly used bioreactors. Its novel vertically rotating wheel allows the propeller to account for more than 80% volume of the U-shaped bottom ensuring homogenous shear rates within the system generated from both radial and axial flow. The unique design of the VWBR impeller allows for efficient mixing at very low shear rates (10-fold lower than traditional stirred tank bioreactors) and have been shown to be applicable to scale up to 500 L. The dynamic system provided by the VWBR has been shown to enhance cell production and reduce manufacturing costs when expanding human iPSCs as aggregates. Along with human iPSC expansion, the VWBR has also been utilized with other cell types for expansion, such as human mesenchymal stem cells (MSCs) and natural killer cells. Differentiation of human iPSCs into pancreatic islets and cerebellar organoids has also been successfully done within the dynamic microenvironment provided by the VWBR and have shown faster differentiation commitment while maintaining high cell quality.

Extracellular vesicles (EVs) are nanoscale, phospholipid-bound particles (30-1000 nm) secreted by cells to mediate intercellular communication through the delivery of bioactive cargo, including proteins, nucleic acids, lipids, and growth factors. A key subpopulation of EVs, exosomes (30-200 nm), are formed via endosomal sorting complex required for transport (ESCRT)-dependent and -independent pathways. Due to their role in cellular communication, EVs exhibit tissue-specific homing capabilities, making them promising candidates for targeted therapeutic drug delivery, including in challenging sites such as the brain. The therapeutic efficacy of EVs depends significantly on their parent cell source and administered dose. Notably, EVs derived from iPSCs demonstrate anti-senescent, anti-inflammatory, and anti-apoptotic properties due to their unique cargo composition. Similarly, EVs obtained from 3D organoids may carry cargo that more accurately mirrors the physiological and structural complexity of human tissues in vivo. However, a major challenge in leveraging EVs for the clinical treatment of brain-related diseases lies in scaling up EV production to meet therapeutic demands. A comprehensive report of 50 preclinical animal studies highlighted a median EV dose of 2.75 mg of EV protein per kg of body weight per administration. Meeting these clinical dose requirements necessitates further research to increase EV generation, enrichment, and establish novel scalable production methods.

The previous studies evaluated the influence of agitation speed on EV generation from human MSCs grown on microcarriers, undifferentiated human iPSCs grown on microcarriers and as aggregates, human iPSC-derived forebrain spheroids, and retinal organoids in VWBRs. The current invention seeks to address the following key questions: How does VWBR affect iPSC differentiation to blood vessel organoids (iBVOs)? How does VWBR affect iBVO EV production and lipid composition? And what are the characteristics and therapeutic effects of iBVO EVs? While EV cargo analysis by proteomics and microRNA-sequencing have been performed previously; lipid profiling remains unexplored. The current application is the first comprehensive study of the lipid profiles of EVs secreted by 3D human iBVOs cultured in a dynamic VWBR system. By providing novel insights into the lipid composition of iBVO EVs and evaluating their therapeutic potential, this research advances the development of dynamic bioreactor systems for scalable organoid-derived EV production as a cell-free therapeutic platform for treating neurological disorders.

### Methods and Materials

### Human IPSC Culture

Human iPSCs (Pluristyx) were thawed from the vial of the Matched Research Grade human iPSC Working Cell Bank (WCB) generated under current good manufacturing practice (cGMP) from Lonza (Walkersville, MD, USA). Human iPSCs were reprogrammed from human CD34+ umbilical cord blood cells using a non-integrating episomal vector reprogramming method (Certificate of Analysis and Lutheran Hospital Institutional Review Board Approval available upon request). Human iPSCs cryogenically preserved in CryoStor CS10 (StemCell Technologies Inc., Vancouver, Canada) were thawed and plated into growth factor reduced Matrigel (BD Biosciences, Franklin Lakes, NJ, USA)-coated Corning CellBIND T-25 cm² and T-75 cm² Cell Culture Flasks (Corning Inc., Corning, NY, USA). Cells were seeded in the presence of Y27632 (10 μM, Sigma-Aldrich, St. Louis, MO, USA) at a density of 15,000 cells/cm² for 3 days as P+1, and 5,000 cells/cm² for 4 days as P+2, in mTeSR media (StemCell Technologies Inc.) and cultured under standard condition (5% CO₂, 37° C.) with daily media change. Cells were passaged after incubation for 7 min with Accutase (StemCell Technologies Inc.). Cells were cultured through 2 passages and then harvested for inoculation of the VerticalWheel bioreactors and 3D static control.

### iBVO Differentiation From Human iPSCs

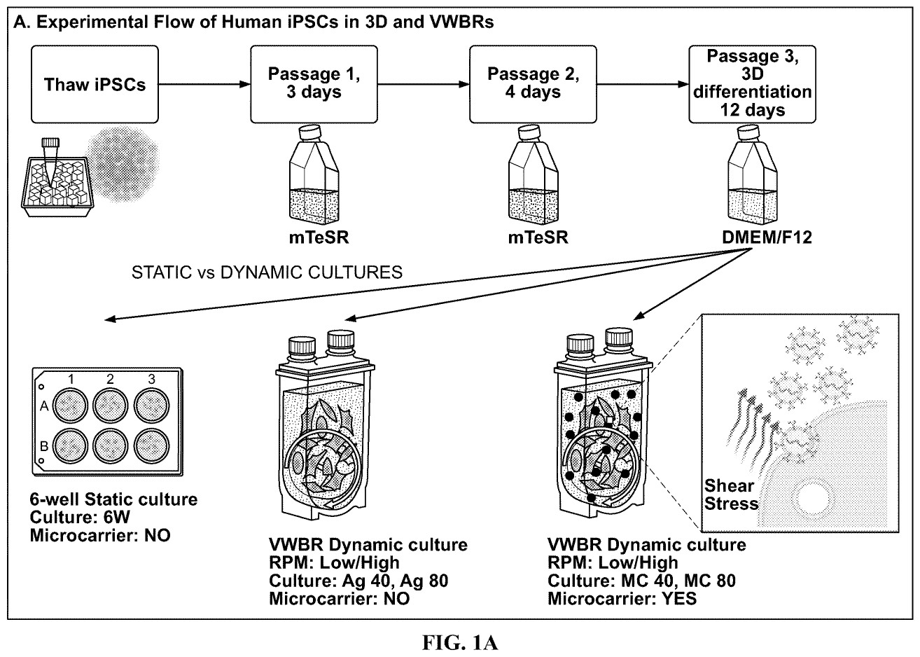

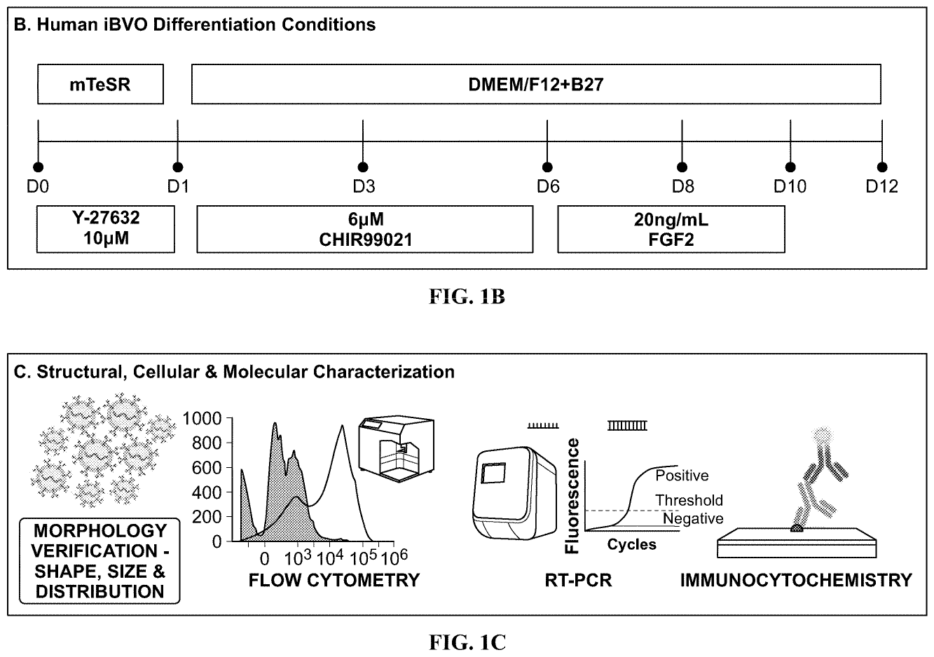

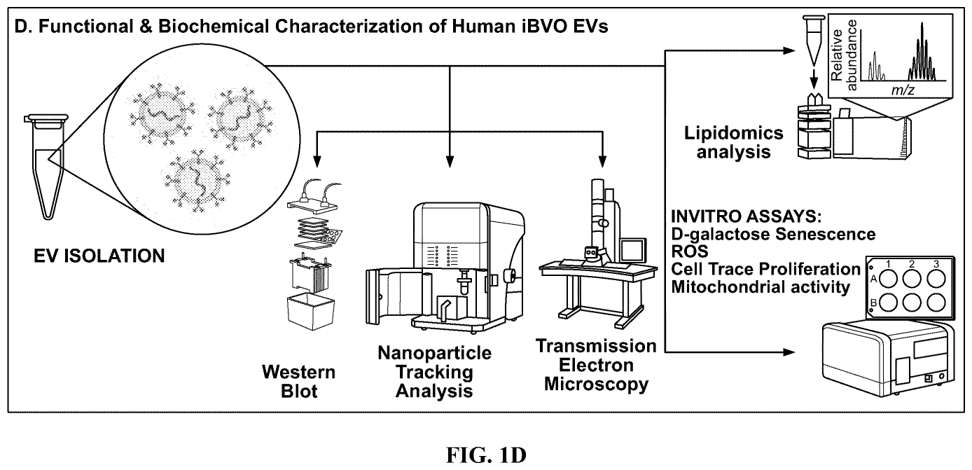

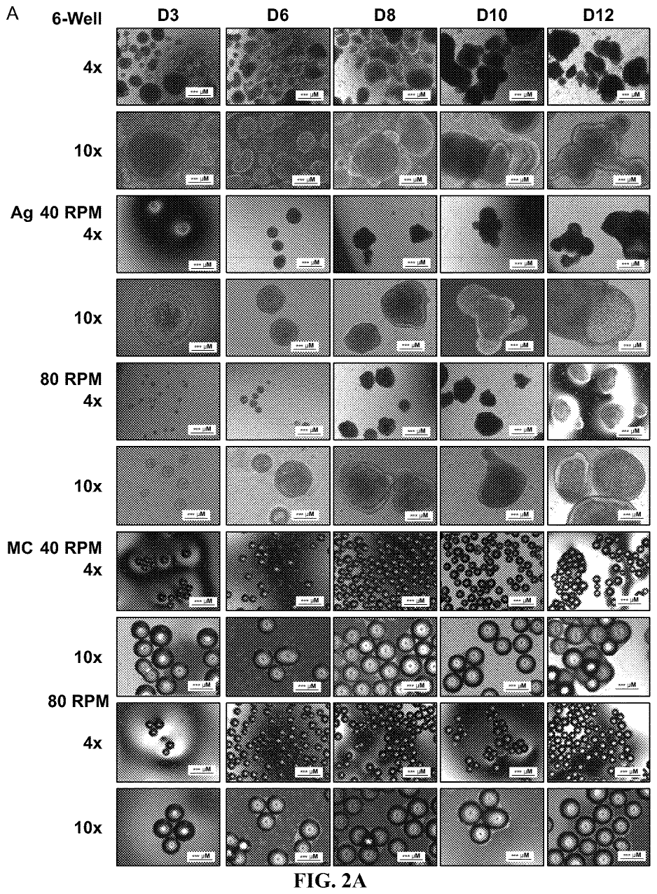

Human iPSCs were harvested for seeding in respective culture conditions. There are five culture conditions: aggregates formed in the VWBR without microcarriers (Ag) at two respective RPMs 40 RPM and 80 RPM (Ag 40 and Ag 80), cells attached to microcarriers that grew in VWBR (MC) at two respective RPMs (MC 40 and MC 80), and aggregates formed in static ultralow attachment 6-well plates without microcarriers (6W). From Day 0 (DO) to D1, the cells were maintained in mTeSR and after D1 cells were cultured in DMEM/F12 plus 2% B27. Media were changed on D1, D3, D6, D8, and D10 and the growth factors were added according to the protocol (FIG. 1B). Cell growth and differentiation was evaluated and D12 spent media were collected for EV isolation.

Microcarrier-based differentiation: Corning® Low Concentration Synthemax® II Microcarriers (360 cm²/gram, Corning Inc.) were added to the Vertical-Wheel bioreactors (PBS Biotech Inc., Camarillo, CA, USA) at a concentration of 20 g/L of working volume. For 100 mL (0.1 L), 2 gram was used, and total surface area was 720 cm² . The bioreactor was inoculated with iPSCs at a seeding density of 8×10⁴ cells/mL, i.e., 1.12×10⁴ cells/cm². The vessels were filled up to the seeding volume with mTeSR media (60 mL for the 0.1 L vessels) in the presence of Y27632 (10 μM). Vessels were maintained at 30 RPM for 24 hours for cell attachment and then raised to 40 or 80 RPM. On day one, 70% of the media were exchanged to DMEM/F12 (Gibco Inc., Billings, MT, USA) containing B27 (Gibco Inc.). Media exchange was performed on D3, D6, D8, D10, and the cells were harvested on D12. On D1, D3, and D6, Wnt activator CHIR99021 (6 μM, Sigma-Aldrich, St. Louis, MO, USA) was added into the media; and on D8 and D10, fibroblast growth factor (FGF)2 (20 ng/mL, PeproTech, Inc., Cranbury, NJ, USA) was added.

Aggregate-based differentiation: The vessels were filled up with 100 mL medium and maintained at 40 RPM or 80 RPM. For the aggregates in ultra-low attachment 6-well plates (Corning Inc.), each well was filled with 3 mL media. Cells equivalent to microcarrier-based bioreactors (8×10⁴ cells/mL) were seeded. The feeding timeline was the same as microcarrier-based differentiation. Three independent runs were performed for microcarrier and aggregate conditions.

### Cell Number and Metabolite Measurements

For each bioreactor, 3 mL of samples were taken out for cell count and metabolite analysis on day 3, 6, 8, 10, and 12. One mL of samples was removed for microscopic observation. The samples were spun at 500 g for 5 min. The supernatant was then removed and frozen for metabolite analysis. The spent media were analyzed with a BioProfile Flex2 (Nova Biomedical, Waltham, MA, USA) analyzer for metabolite concentration. The cells were re-suspended and dissociated by Accutase. The cell suspension was loaded into a Via1-Cassette (ChemoMetec, Bohemia, NY, USA) and put into a NucleoCounter® NC-200 (ChemoMetec, Bohemia, NY, USA) that determined cell concentration. Each measurement was performed with three replicates.

### Cell Harvest, Media Collection, and EV Isolation by ExtraPEG Ultracentrifugation

For bioreactors, microcarriers and aggregates in the media were collected while maintaining 48 RPM agitation. For static cultures, the aggregates were collected in media. After centrifugation at 500 g for 5 min, the culture supernatants were stored at −80 ° C. for EV isolation. The pellets were washed with phosphate buffered saline (PBS) and then dissociated in Accutase. The cells were suspended in FreSR-S cryopreservation medium (StemCell Technologies Inc.) and then stored at −80° C. for further analysis. For EV isolation, ultracentrifugation with ExtraPEG method was used. Conditioned media were sequentially spun (500 g for 5 min, 2000 g for 10 min, 10,000 g for 30 min) to remove cell debris, apoptotic bodies, large vesicles, etc. Polyethylene glycol (PEG)-6000 was added to the supernatant to a final ratio of 8% PEG and 0.5 M NaCl and stored for 24 h at 4° C. in order to enrich EVs. The solution was spun at 3,200 g for one hour and the supernatant was discarded. The remaining pellet was suspended in 1 mL PBS and ultracentrifuged at 127,000 g for 70 min at 4° C. to remove residue PEG. The EV pellet was then suspended in PBS using a benchtop shaker at 1500 rpm for 5 min. EVs were diluted to 10⁸-10⁹ particles per mL in EV-free PBS for nanoparticle tracking analysis.

### Nanoparticle Tracking Analysis (NTA)

NTA was performed on the isolated EV samples in triplicate to determine size distribution and particle concentration, on a NanoSight LM10-HS instrument (Malvern Instruments, Malvern, UK) configured with a blue (488 nm) laser and sCMOS camera. For each replicate, three videos of 60 s were acquired with camera shutter speed fixed at 30.00 ms. To ensure accurate and consistent detection of small particles, the camera level was set to 13, and the detection threshold was maintained at three. The laser chamber was cleaned thoroughly with particle-free water between each sample reading. The collected videos were analyzed using NTA software (version 3.4 Build 3.4.003) to obtain the mode and mean size distribution, as well as the concentration of particles per mL of solution. Compared to the mean size, the mode size is usually a more accurate representation because the vesicle aggregates may affect the mean size.

### Immunocytochemistry

The cell samples from different culture conditions were thawed and replated onto Matrigel-coated 24-well tissue culture plate and allowed to grow for 12 days. The culture medium was aspirated, and the cells were washed with PBS. Then 4% paraformaldehyde (PFA) was added for 20-30 min at room temperature (RT) for cell fixation. For permeabilization required to detect intracellular markers, the fixed cells were washed with PBS and 100% ethanol (EtOH) was added and incubated for 5 min at RT. The EtOH was then aspirated out and the cells were washed with PBS. For nonspecific binding blocking, the PBS was aspirated, and 5% fetal bovine serum (FBS) in PBS (blocking buffer) was added for 30 mins at RT or one hour at 4° C. Then the blocking buffer was aspirated and the cells were incubated in primary antibody diluted in 1% FBS in PBS for 60 min at RT. The samples were washed and then incubated in secondary antibody diluted in 1% FBS in PBS for 60 min at RT in the dark. The cells were washed and then stained with Hoechst 33342 for cell nuclei. The samples were imaged via an epifluorescence microscope with Zeiss Axio Observer 7 with Colibri 7 light source and Hammamatsu Flash 4.0 sCMOS camera using a 20×0.8 NA (Zeiss).

### Flow Cytometry

All cells from each culture condition were dissociated by Accutase, and the single cells in suspension were fixed with 4% PFA for 15 min The samples were resuspended in PBS at 10⁷ cells per mL. Nine volumes of cold 100% methanol were then added, and the cells were incubated on ice for 15-30 min for permeabilization. The samples were spun at 300 g for 5 min, resuspended in blocking buffer, and incubated at 4° C. for 15 min. Primary antibodies prepared in a staining buffer (SB) were then added to the cells and incubated at 4° C. for one hour. After incubation and washing, the secondary antibody was added and incubated at 4° C. for 30-60 min in the dark. After incubation, the cells were washed and resuspended in 150 μL of staining buffer. The samples were acquired with a BD FACSCanto™ II flow cytometer (Becton Dickinson, Franklin Lakes, NJ, USA) and analyzed against isotype controls using FlowJo software.

### Reverse Transcription-quantitative Polymerase Chain Reaction (RT-qPCR)

Total RNA was isolated using TRIzol following the vendor's instructions (Invitrogen). Reverse transcription was carried out using 2 μg of total RNA, anchored oligo-dT primers (Operon) and Superscript III (Invitrogen). Primers for specific target genes were designed using the software Oligo Explorer 1.2 (Genelink). For normalization of expression levels, β-actin was used as an endogenous control. Using SYBR1 Green PCR Master Mix (Applied Biosystems, Foster City, CA, USA), real-time PCR reactions were performed on an ABI7500 instrument (Applied Biosystems). The amplification reactions were performed as follows: 2 min at 50° C., 10 min at 95° C., and 40 cycles of 95° C. for 15 sec, 55° C. for 30 sec, and 68° C. for 30 sec following with a melt curve analysis. The Ct values of the target genes were first normalized to the Ct values of the endogenous control β-actin. The corrected Ct values were then compared to the experimental control. Fold changes in gene expression were calculated using the comparative Ct method: 2^{-(ΔC}_{t treatment}^{-ΔC}_{t control}⁾ to obtain the relative expression levels.

### Western Blot Assay

EV samples were lysed in a radio-immunoprecipitation assay buffer (150 mM sodium chloride, 1.0% Triton X-100, 0.5% sodium deoxycholate, 0.1% sodium dodecyl sulfate, 50 mM Tris, pH 8) with addition of Halt protease inhibitor cocktail (Fisher Scientific, Hampton, NH, USA). The supernatant was collected, and protein lysate concentration was determined and normalized to the lowest sample concentration. Proteins were separated by 12% Bis-Tris-SDS gels and transferred onto a nitrocellulose membrane (Bio-rad, Hercules, CA, USA) for blocking with 5% nonfat dry milk (w/v) in Tris-buffered saline (10 mM Tris-HCl, pH 7.5, and 150 mM NaCl) with 0.1% Tween 20 (v/v) (TBST) and incubated overnight with primary antibody at 4° C. Next, the membranes were washed and incubated with IR secondary antibodies (LI-COR, Lincoln, NE, USA) for three hours at room temperature. Membranes were washed three times in TBST and imaged using the LI-COR Odyssey (LI-COR) system.

### Lipidomics Analysis

Quantitative Lipidomics was performed by Metware Biotechnology Inc. The samples were taken out from the −80° C. refrigerator and thawed on ice. 1 mL of the extraction solvent (MTBE:MeOH =3:1, v/v) containing internal standard mixture was added to the samples. After whirling the mixture for 15 min, 200 μL of ultrapure water was added. The samples were vortexed for 1 min and centrifuged at 12,000 rpm for 10 min. 500 μL of the upper organic layer was collected and evaporated using a vacuum concentrator. The dry extract was dissolved in 200 μL reconstituted solution (ACN:IPA=1:1, v/v) prior to liquid chromatography-tandem mass spectrometry (LC-MS/MS) analysis. The data acquisition instruments consisted of Ultra Performance Liquid Chromatography (UPLC) (Nexera LC-40) and tandem mass spectrometry (MS/MS) (Triple Quad 6500). With in-house database, lipids were annotated based on their retention time and ion-pair information from Multiple Reaction Monitoring (MRM) mode. In MRM mode, the first quadrupole screens the precursor ions for target substance and excluded ions of other molecular weights. After ionization induced by the impact chamber, the precursor ions were fragmented, and a characteristic fragment ion was selected through the third quadrupole to exclude the interference of other non-target ions.

### In Vitro Functional Assays Using a D-Galactose Induced Senescence Model

Human blood vessel organoids (iBVO) were derived from human iPSCs. iBVO from 6-well culture condition were dissociated into single cells by accutase and replated in DMEM/F12+2% B-27. To develop an in vitro senescence model, iBVO were incubated with 150 mM of D-galactose (Sigma) dissolved in PBS. After 48 h, the iBVO were treated with EVs from the five culture conditions (6-well, Ag 80, Ag 40, MC 80, and MC 40) at a dosage of: 2×10⁶ particles/cell to compare with PBS vehicle control. After four days, the iBVO were used for various biochemical assays.

### Biochemical Assays

Reactive oxygen species (ROS) assay: The cells were washed with PBS and treated with 25 μM carboxy-H₂DCFDA (Invitrogen). After 30 minutes incubation at 37° C. (protected from light), the cells were washed using PBS three times, and resuspended in PBS. The fluorescence was measured by a microplate reader (BioRad Laboratories). The images were captured using a Zeiss epifluorescence microscope.

CellTrace cell proliferation assay: The cells were washed with PBS and the assay was performed as described in the manufacturer's protocol (Invitrogen). The CellTrace DMSO stock solution was diluted in PBS and added to the culture. The cells were incubated for 20 minutes at 37° C. Then the solution was removed and the cells were washed twice with culture medium. The cells were incubated with fresh culture medium for 10 more minutes. The fluorescence was measured by a microplate reader. The images were captured using a Zeiss epifluorescence microscope.

MitoSox Green (MSG) mitochondrial activity assay: The cells were washed with PBS and the assay was performed as described in the manufacturer's protocol (Invitrogen). A 1 mM stock solution of MSG was prepared by dissolving the MSG reagent in 10 μL of anhydrous N,N-Dimethylformamide (DMF). A working solution was prepared by adding 10 μL of 1 mM stock to 10 mL of PBS. The cells were incubated with 100 μL per 96 well plate of working solution and incubated for 30 minutes at 37° C. and 5% CO₂. The fluorescence was measured by a microplate reader. The images were captured using a Zeiss epifluorescence microscope.

Transmission Electron Microscopy (tem)

TEM was performed to confirm the morphology of EVs. Briefly, EV isolates were resuspended in 50-100 μL of sterile filtered PBS. For each sample preparation, intact EVs (15 μL) were dropped onto Parafilm. A carbon coated 400 Hex Mesh Copper grid (Electron Microscopy Sciences, EMS) was positioned using forceps with coating side down on top of each drop for one hour. Grids were washed with sterile filtered PBS three times and then the EV samples were fixed for 10 min in 2% PFA (EMS, EM Grade). After washing, the grids were transferred on top of a 20 μL drop of 2.5% glutaraldehyde (EMS, EM Grade) and incubated for 10 min at room temperature. Grid samples were stained for 10 min with 2% uranyl acetate (EMS grade). Then the samples were embedded for 10 min with 0.13% methyl cellulose and 0.4% uranyl acetate. The coated side of the grids were left to dry before imaging on the Transmission Electron Microscope HT7800 (Hitachi, Japan).

### Statistical Analysis

Experimental results were expressed as means ±standard deviation (SD). Statistical comparisons were performed by one-way ANOVA and Tukey's post hoc test for multiple comparisons, and significance was accepted at p<0.05. For comparisons of two conditions, student's t-test was performed for the statistical analysis.

### Example 2: Differentiation of Blood Vessel Organoids From Human iPSCs in the VWBR

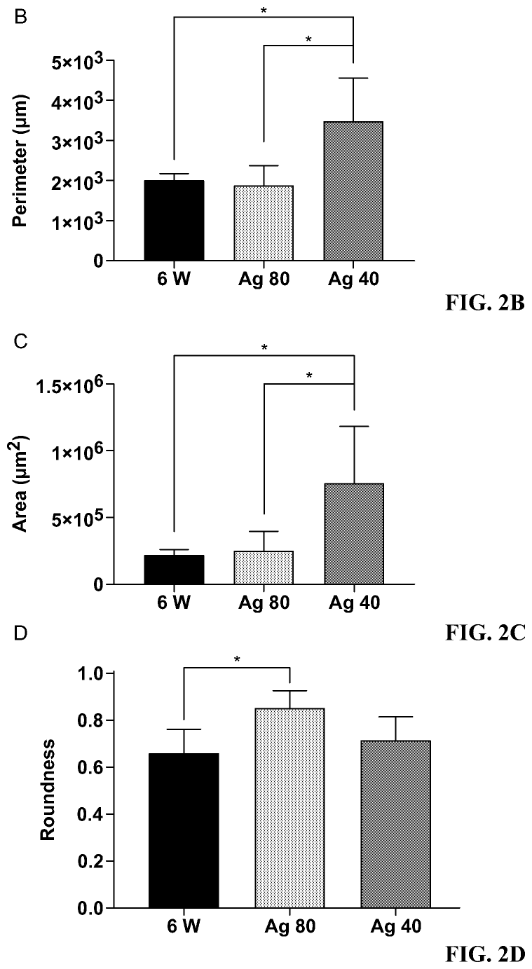

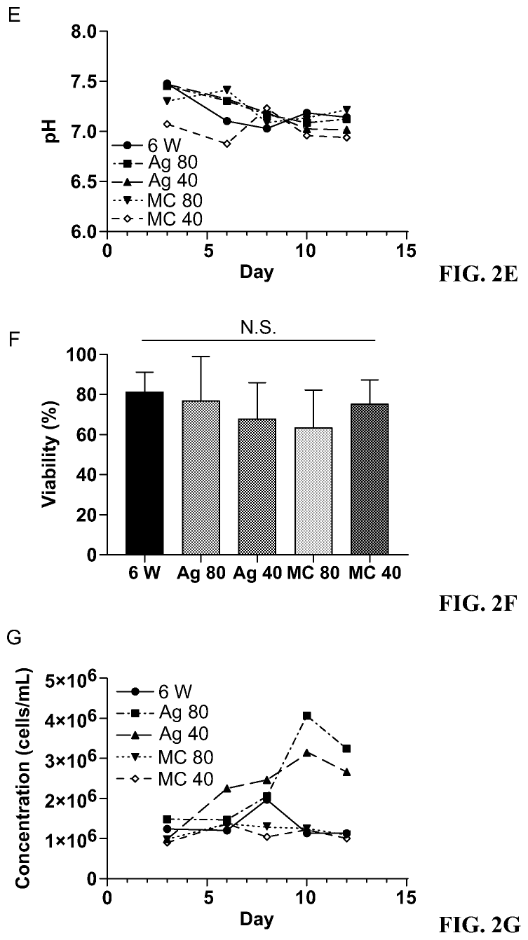

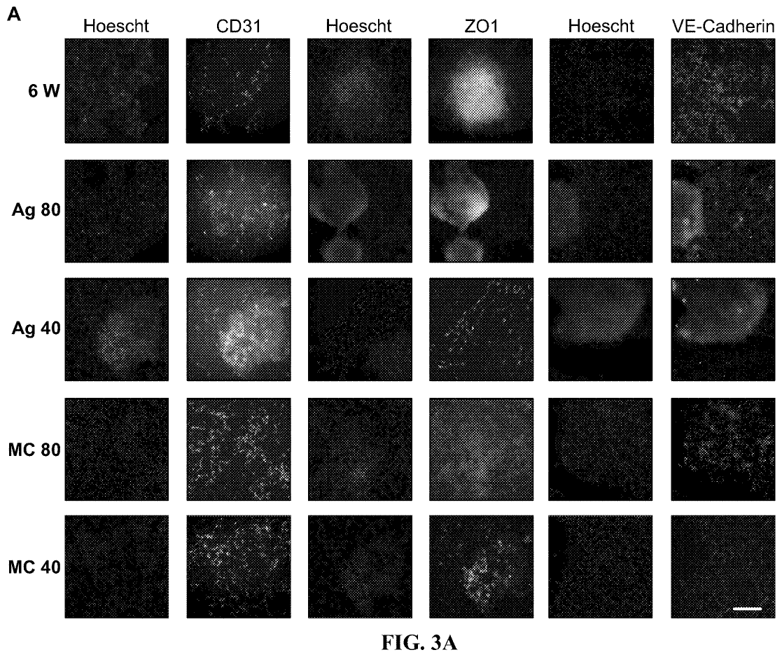

The experimental workflow is depicted in FIGS. 1A-1C. Blood vessel organoids were differentiated from human iPSCs in VWBRs across three independent experimental runs, with data from Run 2 presented as representative results. Morphology images showed that the aggregates significantly increased in size during differentiation (from ~100 μm to ~1000 μm in diameter). By Day 12 (D12), the aggregates in the Ag 40 group exhibited larger perimeters and areas compared to those in the 6-well group (~3000 μm vs. ~2000 μm). In contrast, the aggregates in the Ag 80 group demonstrated perimeter sizes similar to the 6-well group (~2000 μm) (FIGS. 2A, 2B, 2C). The smaller size for the Ag 80 group than the Ag 40 group is probably due to the influence of the shear stress (~0.4 vs. 0.2 dyn/cm² on average). Aggregate merging was observed for the 6-well group, whereas Ag 80 and Ag 40 groups maintained relatively uniform spherical shape (FIG. 2D). Microcarrier-based cultures displayed clumping during cell growth, with the MC 80 group forming larger and more numerous clumps compared to the MC 40 group. pH trends aligned with cell growth, decreasing from 7.5 to 7.0 as cell density increased (FIG. 2E). Cell viability at D12 ranged from 60% to 80%, with no statistically significant differences among the culture conditions, although the MC 80 condition exhibited slightly lower viability compared to aggregate-based conditions (static or dynamic) (FIG. 2F). Growth kinetics demonstrated that dynamic aggregation (Ag 40 and Ag 80) greatly promoted cell growth, reaching 3-4×10⁶ cells/mL and 37- to 50-fold cell number increase over 12 days, while microcarrier conditions sustained growth rate similar to static culture, reaching 1-2×10⁶ cells/mL and yielding 12-25 fold increase in cell number over 12 days (FIG. 2G).

Similar to Run 2, morphological analyses of Runs 1 and 3 demonstrated consistent aggregate growth across both static and dynamic culture conditions. In Run 1, aggregates in the Ag 80 condition exhibited larger areas and perimeters compared to static cultures. However, in Run 3, significant variability in aggregate size across conditions resulted in no statistical differences. For 6-well static cultures, the area, perimeter, and roundness ratio of aggregates were consistent across runs. Notably, the Ag 40 condition showed the greatest variability in area and perimeter sizes, with aggregates in Run 2 being larger than those in Runs 1 and 3, possibly due to the variations of aggregate sampling from VWBR or the sensitivity of the cells to the shear stress. Similar to Run 2, microcarrier cultures in Runs 1 and 3 formed clumps and clusters, with no significant differences in roundness ratios across all conditions or runs. Regardless of the run, pH values followed trends consistent with cell growth, decreasing with higher cell densities. Cell viability showed no significant differences among culture conditions, although Run 1 exhibited the lowest viability overall. In terms of cell number kinetics, Run 1 revealed that Ag 40 achieved the highest cell concentration (~1.6-2.0×10⁶ cells/mL), while Ag 80 and MC 80 were comparable to static controls (~1.2×10⁶ cells/mL). MC 40 showed the lowest cell concentration. In Run 3, by day 12, Ag 40 and Ag 80 maintained higher cell concentrations (~2×10⁶ cells/mL) than static controls (~1.2×10⁶ cells/mL), whereas MC conditions were comparable to or slightly below static controls (~1.0×10⁶ cells/mL). Taken together, the aggregate culture of VWBR had similar or larger size (1.0-1.5 fold) of aggregates than static control and the cell numbers were 1.3-2.0 fold higher across all runs. The MC cultures did not show higher cell number than static control in general, possibly due to the influence of shear stress in VWBR.

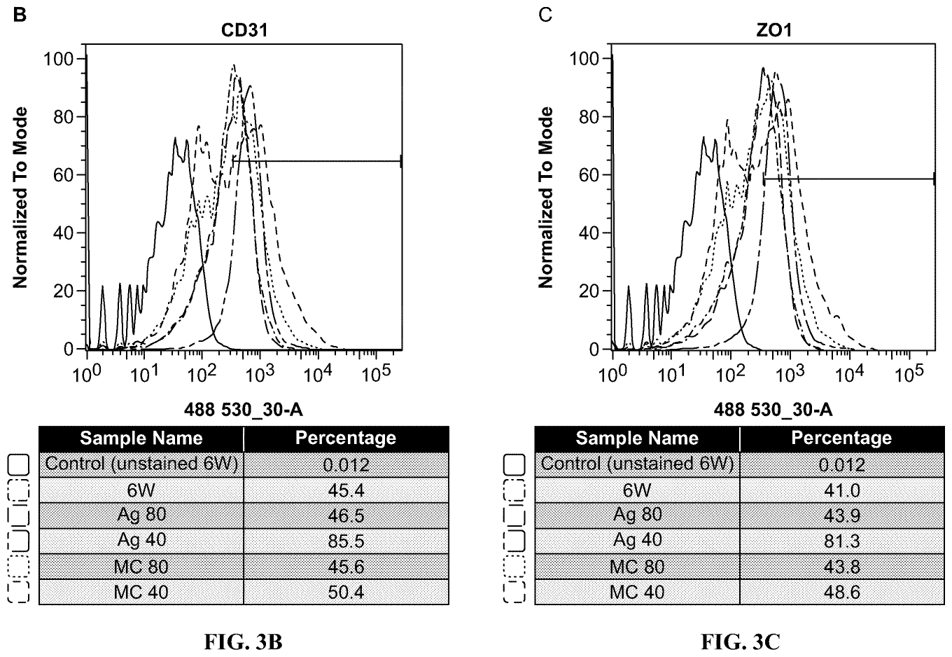

Differentiation marker expression was evaluated on Day 24 cells following the thawing and replating of cryopreserved Day 12 cells. Immunocytochemistry analysis confirmed the presence of key iBVO markers across all culture conditions. These markers included CD31, an endothelial cell marker indicative of blood vessel lining, ZO-1, associated with tight junction maintenance, and VE-Cadherin, a later-stage endothelial maturation marker (FIG. 3A). Flow cytometry further quantified the expression of CD31 and ZO-1. Most conditions exhibited moderate levels of CD31 and ZO-1 expression (45.4%-50.4%). Notably, the Ag 40 condition demonstrated the highest expression levels, with CD31 and ZO-1 reaching 85.5% and 81.3%, respectively (FIG. 3B). Gene expression analysis via RT-qPCR showed upregulation of ZO-1 in all conditions except MC 40, compared to the 6-well control. VEGF expression remained stable across conditions, with a slight downregulation in Ag 80. CD105 showed slight downregulation in MC 80 and MC 40, while Ag 80 exhibited modest upregulation. Gene expression analysis for Ag 40 could not be performed due to insufficient cell samples. Taken together, these findings confirm that VWBR cultures supported the differentiation and expression of essential iBVO markers, highlighting the potential for generating blood vessel organoids.

### Example 3: Metabolite Analysis of VWBR Based Differentiation

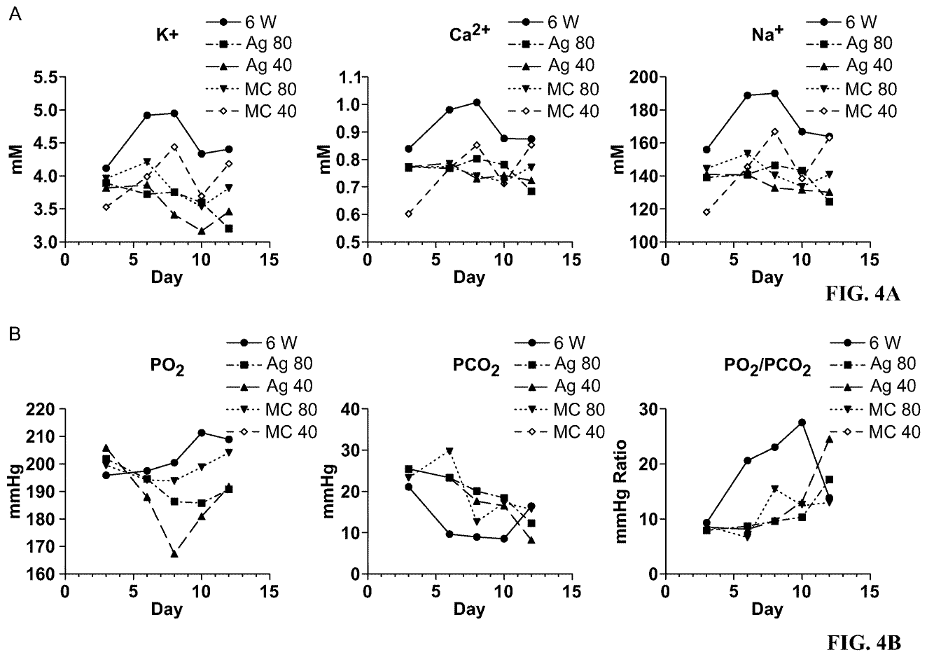

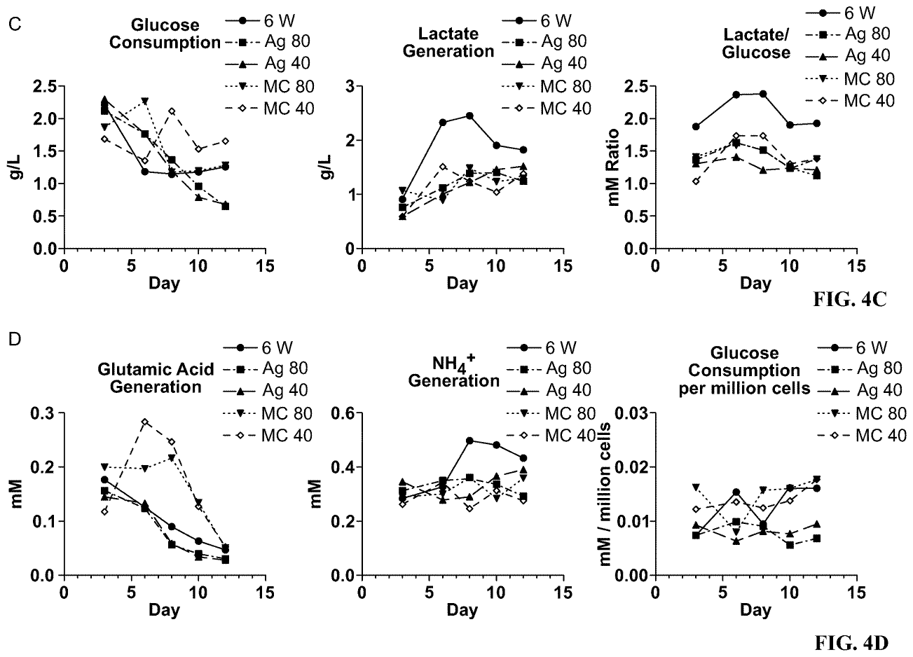

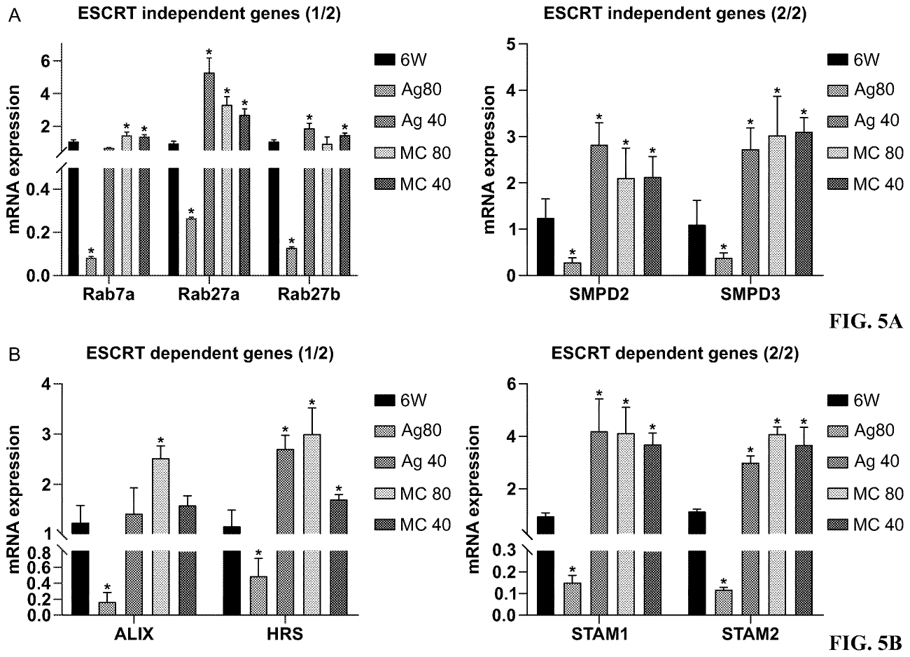

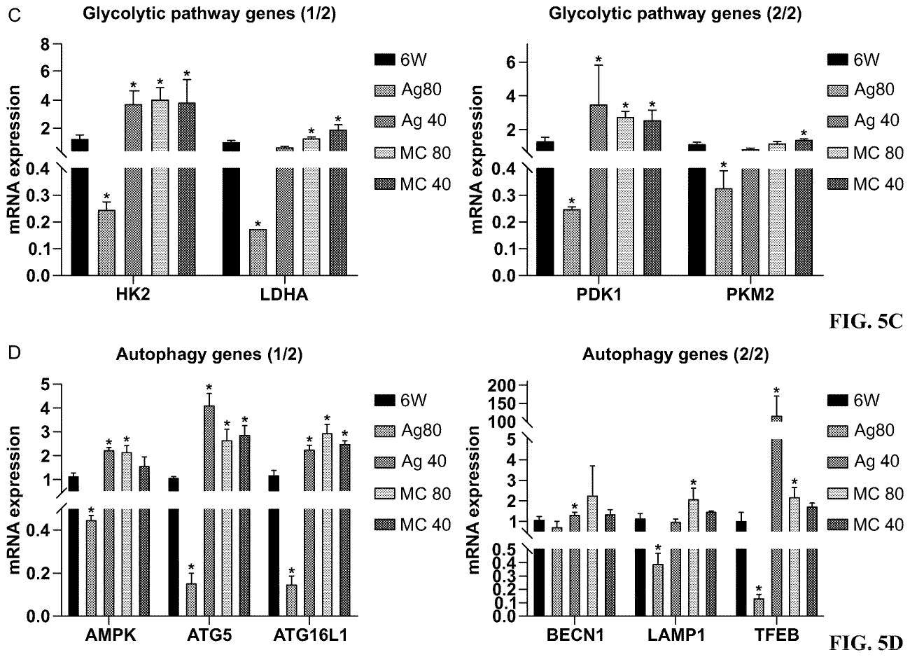

Metabolite analysis was performed on the spent media of different culture conditions. The results showed that all culture conditions maintained normal K+, Ca2+, and Na+ concentration levels, but the 6-well conditions consistently had higher concentrations of K+, Ca2+, and Na+ (FIG. 4A). PO2 (partial pressure of oxygen) levels for all culture conditions were high (ambient air pressures, ranging from ~170 mmHg to ~210 mmHg), but could be attributed to excess gas exchange due to exposure to free air prior to measurement. The 6-well static control had higher values after day 5 than other VWBR conditions. For the PCO2 levels, the values were lower than the optimal 35-45 mmHg range for typical cell culture, also possibly due to the exposure to free air prior to measurement (FIG. 4B). The 6-well static control had lower values before day 12 than other VWBR conditions. Both aggregate conditions (Ag 80 and Ag 40) had the highest glucose consumption rate (e.g., lowest glucose concentration after day 8). MC 40 and 6-well group had medium glucose consumption, and MC 80 had the lowest glucose consumption, which is consistent with the pattern of the cell growth kinetics (FIG. 4C). For the lactate production, the 6-well plate static control had the highest values, reaching 2 g/L while the other VWBR conditions had similar lactate concentration lower than 1.5 g/L. When the mole ratio of lactate to glucose was calculated, all culture conditions showed values between 1.0-2.5 (FIG. 4C). The 6-well plate static control had the highest values of 2.0-2.5, indicating anaerobic metabolism. All conditions typically increased in ratio over time, indicating more anaerobic metabolism when cell number increased. Ag 40 condition showed the lowest ratio of 1.2-1.3 in general, indicating more aerobic metabolism. All other VWBR conditions had the values of 1.3-1.7. Similarly to glucose consumption, glutamic acid decreased over time for all culture conditions. Ammonium generation was consistent and similar for all culture conditions except the 6 well condition that experienced an increase in generation from D8 onward (FIG. 4D). Glutamine was not able to be measured due to the use of Glutamax. When glucose consumption was normalized to cell number, Ag 40 and Ag 80 had the lower values after day 8 compared to other conditions. Taken together, VWBR cultures, in particular the aggregate controls, exhibited different metabolic pathways compared to static 6-well plate control. Run 3 was more similar to Run 2 than Run 1 (there was one day power outrage around day 8 in Run 1). Ca++, K+, and Na+ concentration levels were normal for all runs. The trend of the metabolites was similar with these ions, with the 6 well condition having the highest concentrations while the other VWBR conditions were similar to each other. PO2 levels for all culture conditions were high (ambient air pressures, ranging from ~170 mmHg to ~250 mmHg) but were consistent with Run 2. The same trend could be found in the PCO2 levels which were lower than the optimal 35-45 mmHg range for typical cell culture but consistent to the values found in Run 2. For glucose metabolism, the lactate to glucose mole/mole ratio was the highest for 6-well plate static control with values of 2-3, indicating the anaerobic metabolism. All other VWBR conditions had values around 1.5 due to more aerobic metabolism. The glutamine metabolism showed the decreased glutamic acid and ammoniμm generation along the differentiation.

Example 4: EV biogenesis, glycolysis, and autophagy analysis Gene expression of cells at D12 was determined for all the conditions. For EV biogenesis in ESCRT-independent pathways, Ag40, MC80, and MC40 groups showed significantly higher expression of Rab7a, by 5-fold, 3-fold, and 2-fold respectively compared to the static control (FIG. 5A). For Rab27b, Ag 40 and Mc 40 conditions showed 2-fold increase compared to the control. Rab7a showed little slightly increase for MC80 and MC40. For SMPD2, Ag40, MC80, and MC40 groups showed significantly higher expression, by 2.8-fold, 2-fold, and 2-fold respectively compared to the static control. Similarly, Ag40, MC80, and MC40 groups showed significantly higher expression than the static control by 3-fold for SMPD3. Ag80 condition had significantly lower expression than all the other conditions for all the markers. For ESCRT dependent pathway (FIG. 5B), only MC80 showed higher ALIX expression than the static control. For HRS, Ag40 and MC80 had 2.8-3.0 fold higher expression than the static control, while MC 40 had 1.5 fold higher expression. For STAM1, Ag40, MC80, and MC40 groups showed significantly higher expression than the static control by 4-fold. Similarly, Ag40, MC80 and MC40 had 3-4 fold higher STAM2 expression than the 6-well static group. Somehow, Ag 80 showed lower expression than all the other conditions for all the markers. This may be related to the mRNA sample preparation or the variations of the collected cell samples of Ag80 group due to aggregate heterogeneity. Taken together, Ag 40 and MC groups of VWBR cultures promoted the expression of EV biogenesis genes.

The expression of glycolytic activity genes was determined. Ag40, MC80 and MC40 had ~4-fold higher HK2 expression and 2-fold higher PDK1 than the 6-well static group (FIG. 5C). MC80 and MC40 had slightly higher (1.5-2 fold) LDHA expression and PKM2 was slightly higher for MC 40 than the 6-well group. The Ag 80 condition showed low expression for all the markers again (FIG. 5C). For cellular autophagy, AMPK (1.5-2.2 fold), ATG5 (3-4 fold), and ATG16LI (2-3 fold) were significantly higher for Ag40, MC80 and MC40 compared to the static control (FIG. 5D). Ag40 was slightly higher for BECN1, and MC80 was slightly higher for LAMP1, For TFEB, Ag40 was significantly higher (>100-fold) and MC80 was 2-fold higher than static control. The upregulation of glycolytic genes and the genes responsible for regulation of autophagy was consistent with the previous study for hMSC EV production in VWBR.

### Example 5: EV Isolation and Characterization

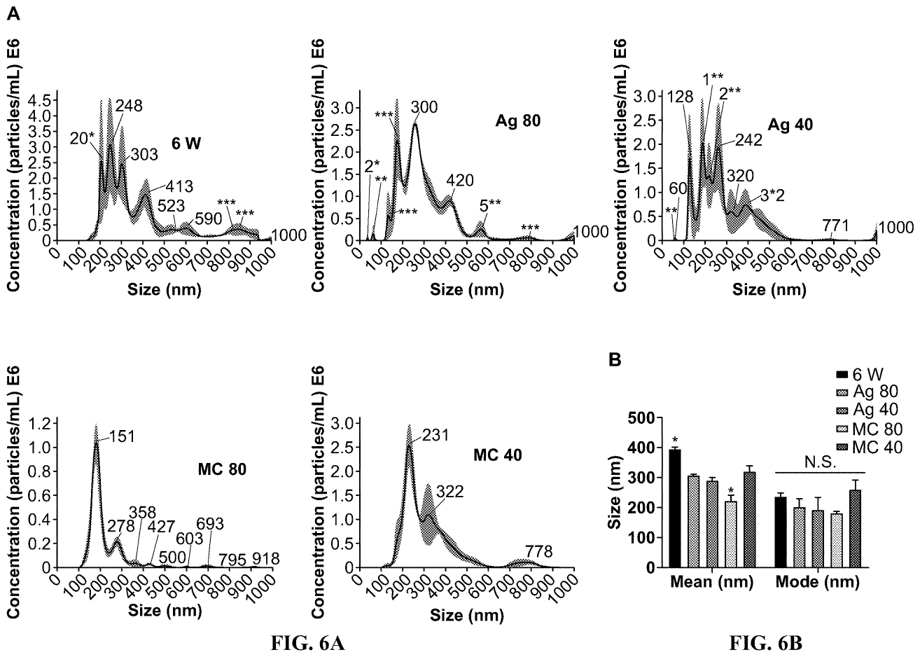

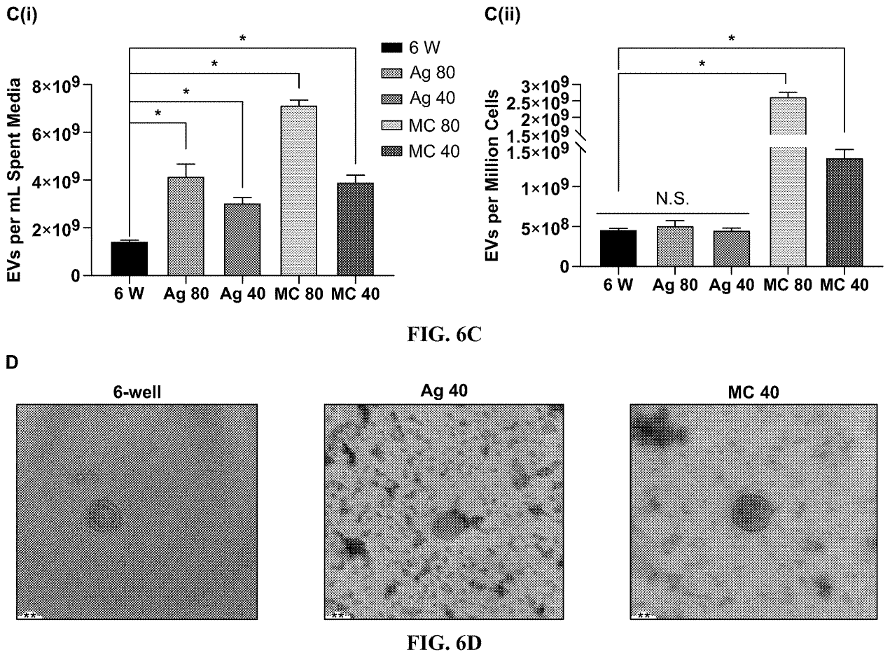

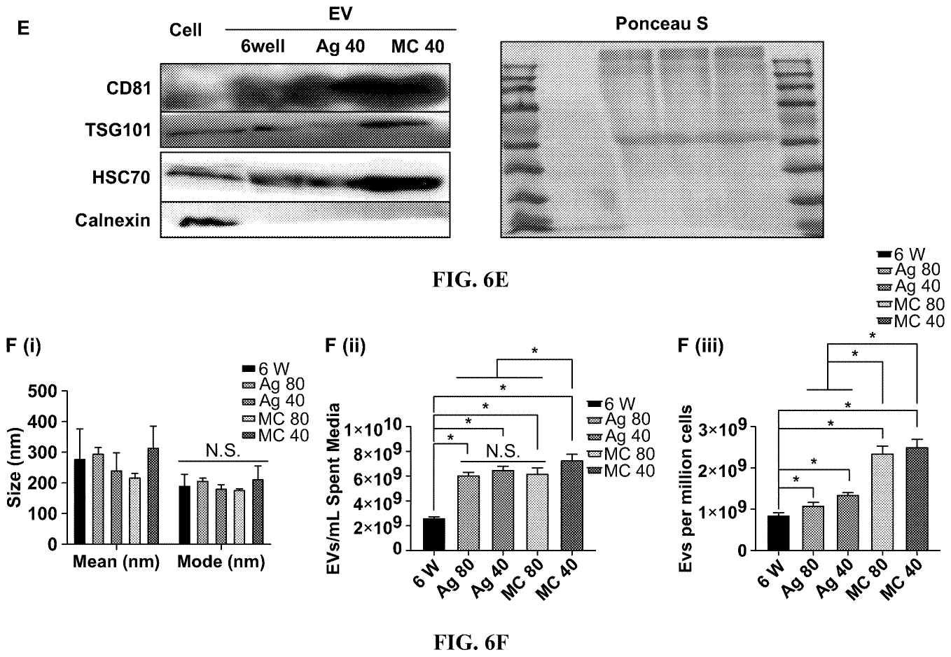

EVs were isolated from D12 media of all culture conditions. The EV size and concentrations were measured by NTA, and the representative EV size distributions from Run 2 are shown in FIG. 6A. The mean sizes for all conditions were around 250-400 nm (FIG. 6B). The mode sizes were around 200 nm, with Ag 40 and MC 80 having lower value of around 180 nm (in exosome size range). The static 6-well culture condition showed a slightly larger mean size (~230 nm) as well as and the MC 40 condition (~270 nm). The mode size is a more accurate representation because aggregation may affect the mean size. EV yields were calculated by normalizing EV numbers to spent media and cell number (FIG. 6C). The 6-well condition had the lowest yield when normalized by volume of spent media. Ag 40, Ag 80, and MC40 had 2-3 fold higher EVs than the 6-well plate control. MC80 had ~5 fold higher EV numbers possibly due to easy EV release from cells and higher shear stress. When normalized per million cells, both microcarrier conditions exhibited significantly higher yields (4.7-fold of the 6-well for MC 80; 2-fold of the 6-well for MC 40). Static and dynamic aggregate conditions (6-Well, Ag 80 and Ag 40) showed similar yields, possibly due to EV retention inside the ECM network of aggregates. TEM images showed that these EVs presented doubled-layered cup-shaped morphology for all the conditions (FIG. 6D). Only the low RPM groups (the preferred conditions) and the static condition were used for confirmation of exosomal markers (FIG. 6E). Calnexin (the negative exosome marker) was enriched in cell lysates, but not shown in the EVs. HSC70, TSG101, and CD81 are positive exosome markers that were shown in the EVs from all three culture conditions.

Similar to Run 2, the representative size distributions of Run 1 and Run 3 indicated that the majority of EVs was within the range of exosome size (~200 nm). Similar mode sizes between culture conditions and between runs were observed. Yield calculation with EVs per mL media show that consistently, across all runs that the 6-well condition yielded the lowest number of EVs. The VWBR conditions showed 2-6 fold higher EVs per mL of spent media than that of the static 6-well culture. When yield was normalized by EVs per million cells, similar or higher yields were observed for Ag 40 and Ag 80, while the MC 80 and MC40 were always higher than the 6-well condition. When the results of all the three runs were combined, the EV mode size was similar for all the conditions (FIG. 6F). For EV number per mL spent media, which reflects the total EV production, the VWBR conditions secreted 2-3 fold higher EVs than static 6-well control. When normalized to cell number, which reflect EV release mechanism, the MC 80 and MC40 had 2-3 fold higher EVs than static 6-well control and slightly higher yields were observed for Ag 40 and Ag 80. Taken together, VWBRs significantly promoted total EV secretion.

### Example 6: Lipidomics Analysis of EV Lipid Composition

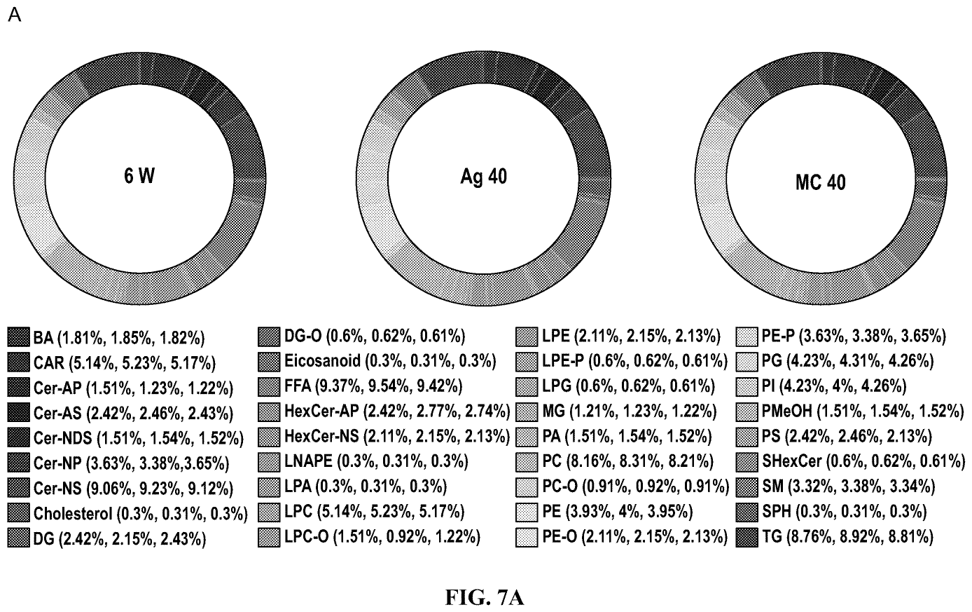

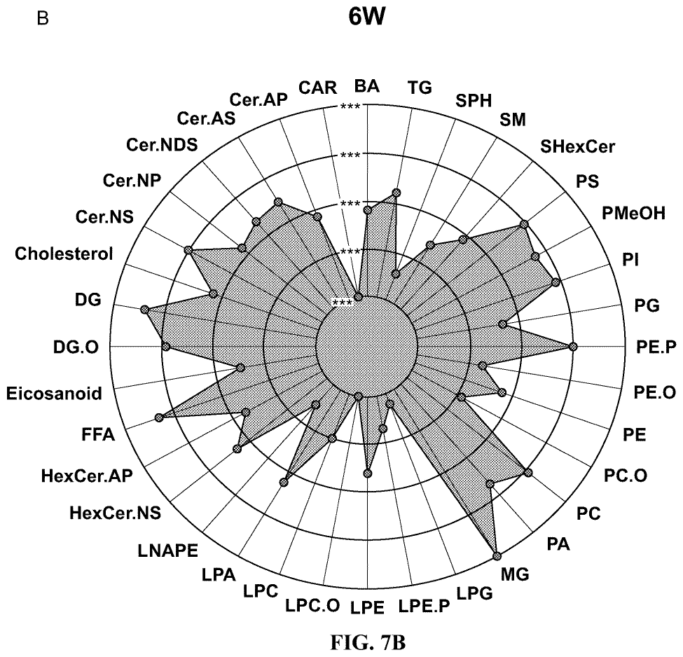

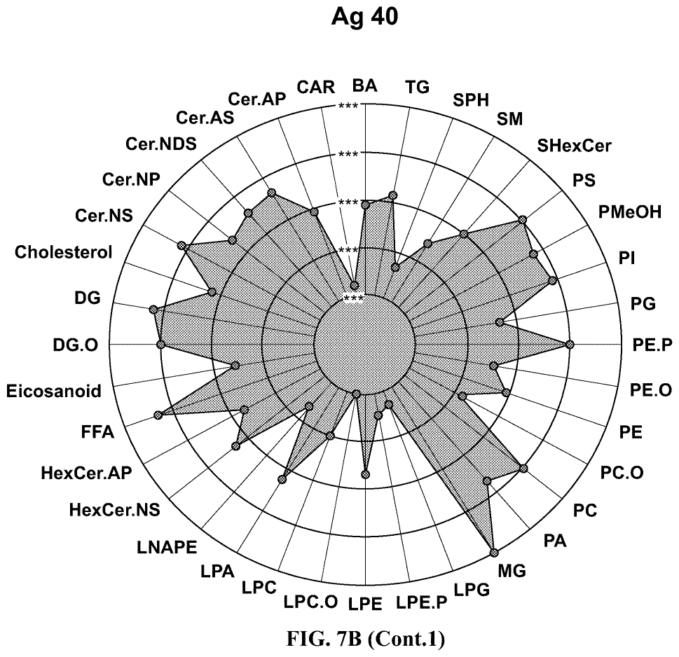

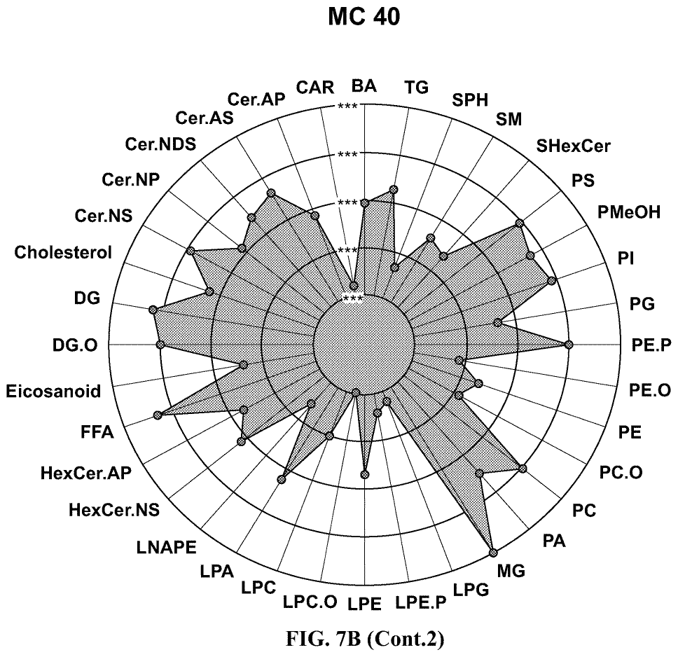

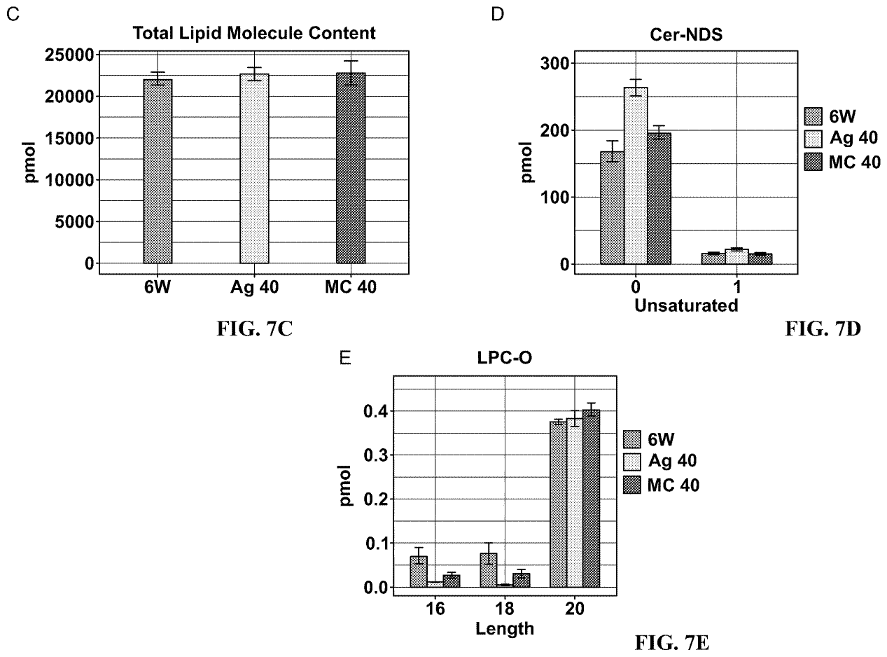

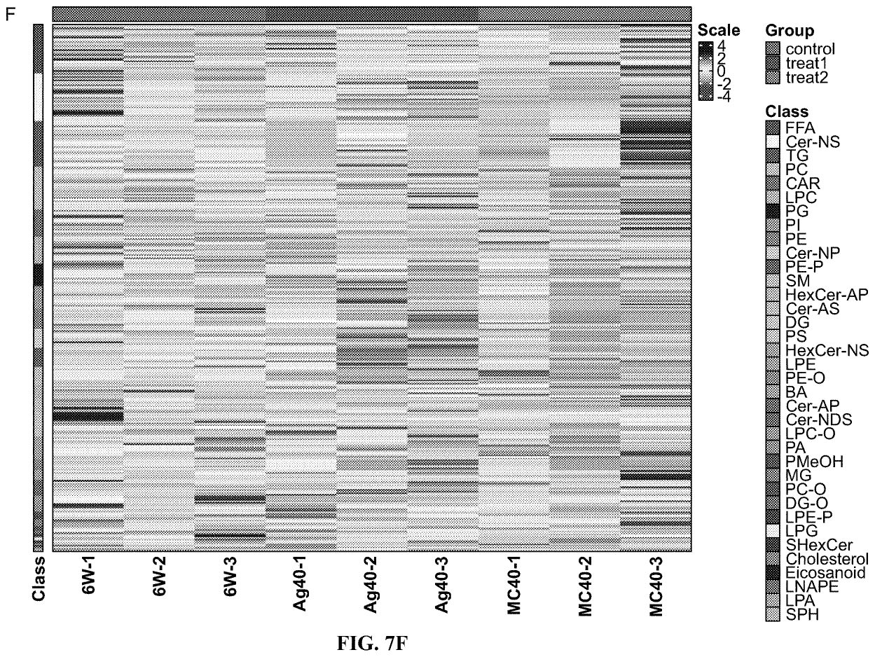

Lipidomic analysis reveals the lipid composition of the EVs from 6-well, Ag 40, and MC 40 conditions and the results showed that lipid subclasses were all comparable, with content varying less than 0.3% between each culture condition (FIG. 7A). A radar plot further visualized this while showing that the lipid subclasses most prevalent in content are MG (monoglycerides), FFA (Free Fatty Acids), DG (Diglycerides), PC (phosphatidylcholine), PMeoH (phosphatidylmethanol) and, Cer-NS (ceramide-non-substituted lipids) (FIG. 7B). All samples analyzed had similar lipid subclass compositions and similar total lipid molecule content (FIG. 7C). Within certain subclasses there are statistically significant differences within different conditions, e.g., chain unsaturation. Chain unsaturation is the number of double bonds in the fatty acid chain. The abundance of lipid compounds with the same number of unsaturated bonds between sample groups can vary such as in the Cer-NDS group where Ag 40 had the most abundance of Cer-NDS with both zero and one double bond, MC 40 as the median, and 6-well with the least abundance (FIG. 7D). Cer-NDS can play a crucial role in various cellular processes, including cell signaling, apoptosis (cell death), and cell differentiation. Saturation can affect substrate specificity, membrane properties, cellular signaling and regulatory mechanisms. Chain length is another property within subclasses that can have statistically significant differences between samples. Chain length can affect permeability of lipid membranes due to membrane thickness, and melting temperatures which also contribute to membrane fluidity. LPC-O is a lipid subclass that had statistically significant chain length differences between samples with the 6-well group being the highest, followed by MC 40, and Ag 40 as the lowest (FIG. 7E). This trend held true for both 16 and 18 length carbon chains of LPC-O but there was no statistical difference in abundance with the 20-carbon chain LPC-O. LPC-O plays roles in membrane structure and integrity, lipid metabolism and cell signaling. The chain length of LPC-O can affect their metabolism via reaction kinetics changing product formation, upregulating or downregulating cellular functions influenced by LPC-O such as proliferation, wound healing, and migration. Heatmap analysis reveals the differences among the three conditions, where each condition had its unique lipid profiles (FIG. 7F).

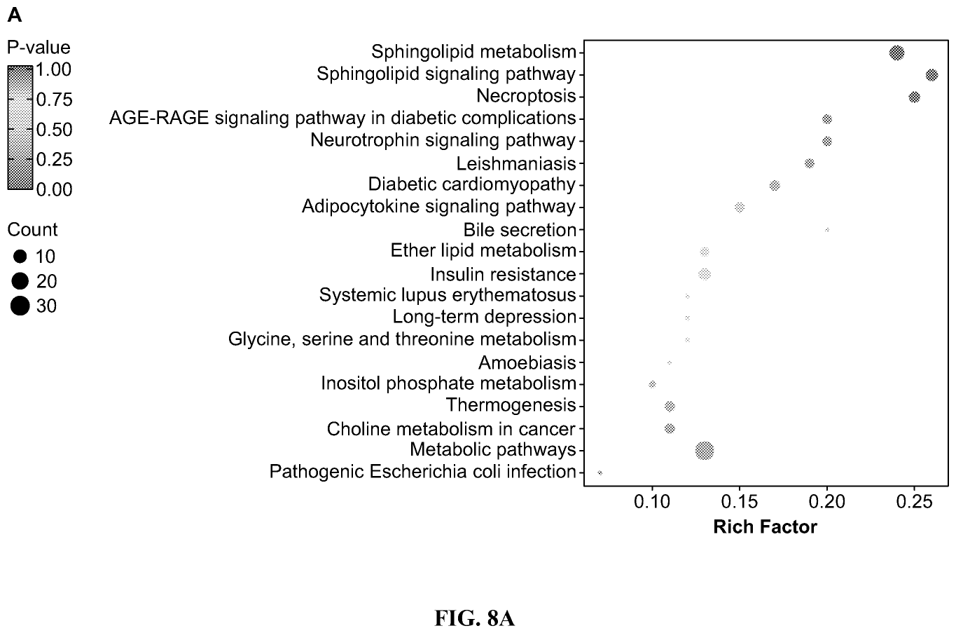

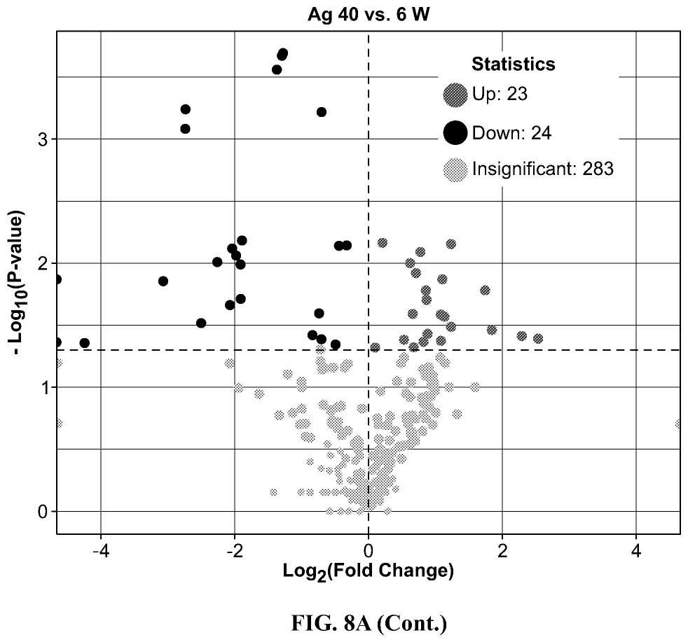

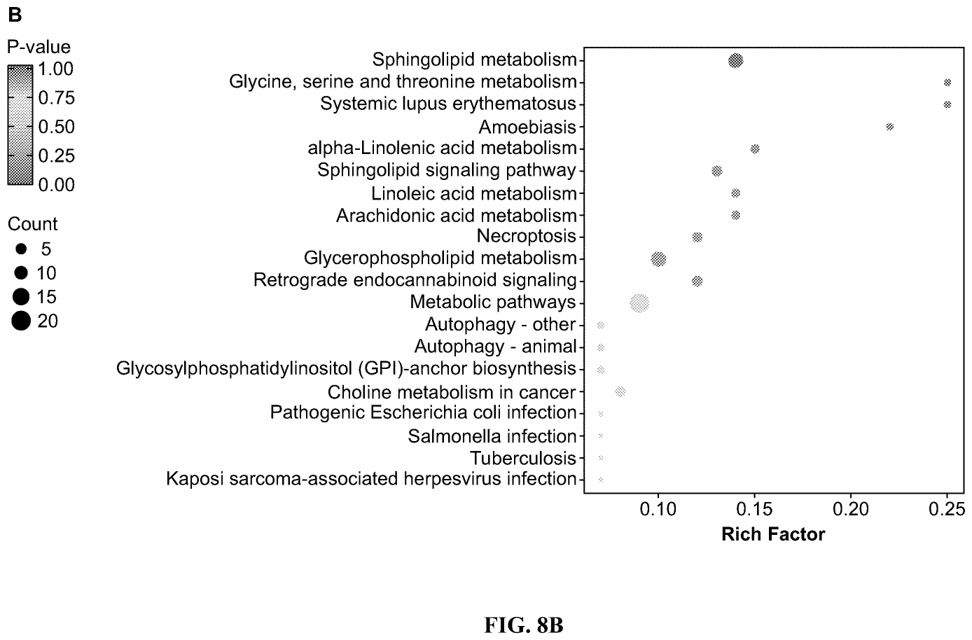

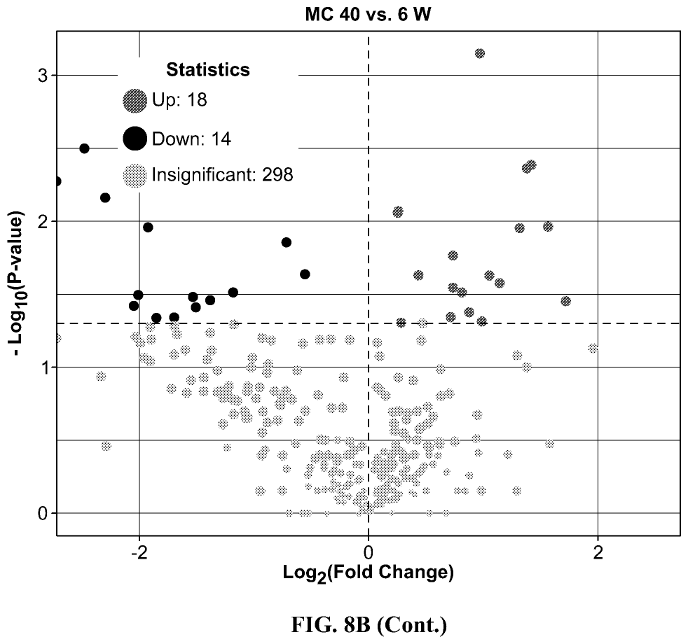

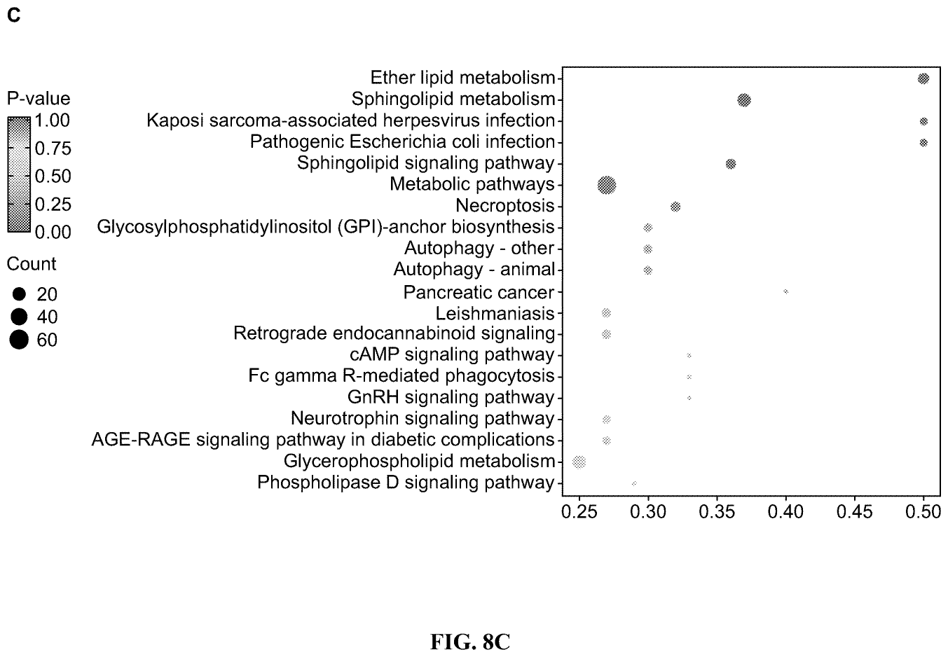

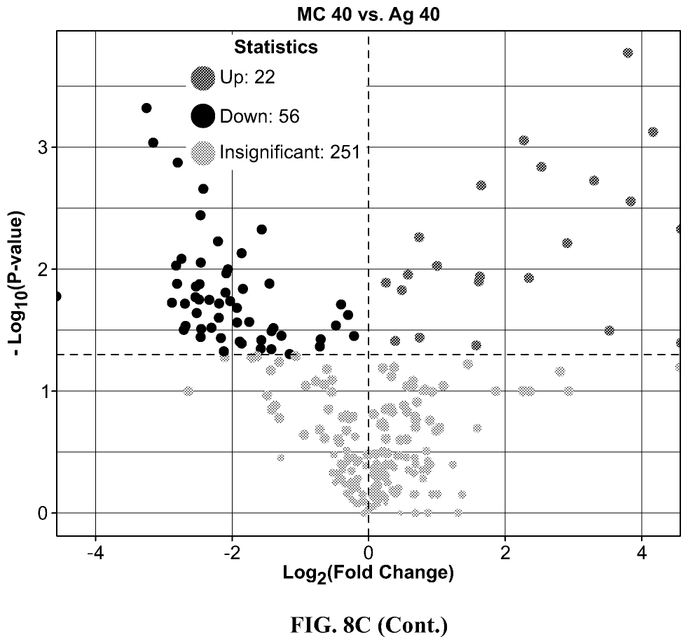

The differentially expressed lipids (DELs) were compared between Ag 40 vs. 6-well and shown in Volcano plot (FIG. 8A). There were 23 upregulated lipids (e.g., 2 carnitine lipids, 12ceramide lipids, 1 free fatty acid lipid, 2 phosphatidylethanolamine lipids, 2 phosphatidylglycerol lipids, 1 phosphatidylinositol lipid, 1 phosphatidyl-myo-inositol ethyl ester lipid, 1phosphatidylserine lipid, and 1 sphingomyelin lipid) and 24 downregulated lipids (e.g., 4 carnitine lipids, 3 ceramide lipids, 1 diacylglycerol lipid, 1 hexosylceramide lipid, 9 lysophosphatidylcholine lipids, 1 phosphatidylglycerol lipid, 3 sphingomyelin lipids and 2 triglyceride lipids) in Ag 40. The most upregulated lipids were in the ceramide subclass which regulate cell growth, differentiation, structural integrity and membrane fluidity, as well as apoptosis and inflammation regulation. The most downregulated lipids were in the lysophosphatidylcholine subclass which can act as a pro-inflammatory mediator for neuroinflammation, promoting tumor progression and endothelial dysfunction. The Kyoto Encyclopedia of Genes and Genomes (KEGG) pathway analysis showed the top 20 significantly different lipids enriched in the corresponding pathways, such as Sphingolipid metabolism and signaling pathway, AGE-RAGE signaling pathway, Necroptosis, and Neurotrophin signaling pathway (FIG. 8A).

A similar differential lipid comparison was performed between MC 40 vs. 6-well. There were 18 upregulated lipids (e.g., 2 carnitine lipids, 3 ceramide lipids, 4 hexosylceramide lipids, 2 phosphatidylcholine lipids, 1 phosphatidylglycerol lipid, 5 sphingomyelin lipids and 1 sphingolipid) and 14 downregulated lipids (e.g., 1 carnitine lipid, 2 ceramide lipids, 1 lysophosphatidylcholine lipid, 2 phosphatidylethanolamine lipids, 2 phosphatidylcholine lipids, 2 phosphatidylethanolamine lipids, 1 phosphatidylinositol lipid, 2 phosphatidylserine lipids, and 1 sulfated hexosyl ceramide lipid) in MC 40. The most upregulated lipids were in the sphingomyelin subclass which have been shown to be essential in preventing loss of synaptic plasticity, cell death and neurodegeneration. Previous studies have shown that sphingomyelin plays key roles in the processing and aggregation of common neuroinflammation agent Aβ utilizing models to mimic exosomes, linking unregulated sphingomyelin to Alzheimer's formation and proliferation. There was no subclass of lipids that was significantly downregulated with respect to the other downregulated subclasses. The top 20 significantly different lipids enriched are related to the Sphingolipid metabolism and signaling pathway, Glycine, serine, and threonine metabolism, Linoleic acid metabolism etc. (FIG. 8B).

A differential lipid comparison was also performed between MC 40 vs. Ag 40. There were 22 upregulated lipids (e.g., 3 carnitine lipids, 6 ceramide lipids, 2 diacylglycerol lipids, 1 hexosylceramide lipid, 4 lysophosphatidylcholine lipids, 1 phosphatidylcholine lipids, 1 phosphatidylethanolamine lipid and 4 sphingomyelinlipids) and 56 downregulated lipids (e.g., 15 ceramide lipids, 1 free fatty acid lipid, 1 hexosylceramide lipid, 1 lysophosphatidylcholine lipid, 2 phosphatidylethanolamine lipids, 1 lysophosphatidylglycerol lipid, 2 phosphatidic acid lipids, 3 phosphatidylcholine lipids, 21 phosphatidylethanolamine lipids, 3 phosphatidylglycerol lipids, 1 phosphatidylinositol lipid, 2 phosphatidylserine, 3 sulfated hexosyl ceramide lipids and 1 sphingomyelin lipid) in MC 40. The subclass with one of the most upregulated lipids was in the lysophosphatidylcholine subclass, which is the key to the integrity of cell membrane and has also been linked to neuronal damage, cell loss, and neurodegenerative diseases such as Alzheimer's and Parkinson's disease. The most downregulated lipids were in the phosphatidylethanolamine subclass which is involved in vesicle formation and neurotransmission. The top 20 significantly different lipids enriched are related to the Ether lipid metabolism, Sphingolipid metabolism and signaling pathway, Metabolic pathways, Necroptosis, etc. (FIG. 8C). These findings indicate that there is difference related to Necroptosis and Metabolic pathways between bioreactor cultures and static control. Given the enhanced oxygen and nutrient delivery of dynamic cultures, the static control had higher level of necroptosis.

Principal component analysis (PCA) plot showed the distinct clusters of Ag 40 EVs while 6 well and MC 40 EVs had wider spread clusters. The correlation network diagrams of DELs displayed the degree of connection of differential lipids between EVs. The dots in the FIGS. 8A, 8B, and 8C represent the various differential lipids, and the size of the dot is related to the Degree of connection. The larger the dot, the greater the Degree of connection, i.e., the more dots (neighbors) connected to it. Red lines represent positive correlations, and blue lines represent negative correlations. Compared to the 6 well EVs, the Ag 40 EVs had more differential lipids, with significantly more negative correlations than that of the MC 40 EVs. Comparing MC 40 EVs to Ag 40 EVs yielded the largest degree of connections with multiple positive and negative correlations. The differential lipid regulation network diagrams were built using KEGG pathways to create regulatory interaction networks. Red dots represent a metabolic pathway, yellow dots represent enzymes, green dots represent a metabolic pathway of background material, purple dot represents molecule modules, and blue dot represents a chemical interactions reaction. The significant DELs were classified based on pathway annotation and the number of lipids and the proportion of the total lipids are plotted to a bar plot. The most differential KEGG pathways among EVs were all different but what stayed consistent between EVs is the high percentage of metabolism pathway lipids that make up total lipid count. Taken together, the differentially expressed lipids in the EVs of the three culture conditions are mainly related to Sphingolipid metabolism and signaling pathway and metabolism regulation.

### Example 7: In Vitro Functional Analysis

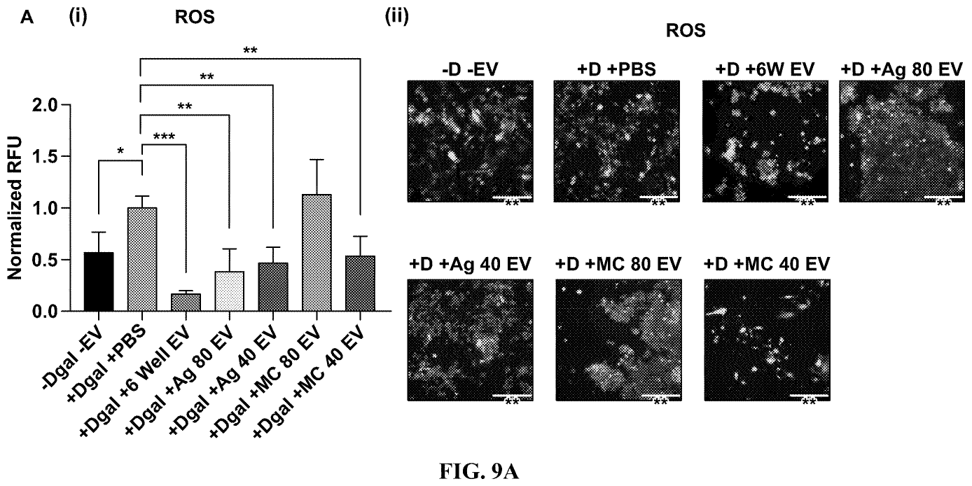

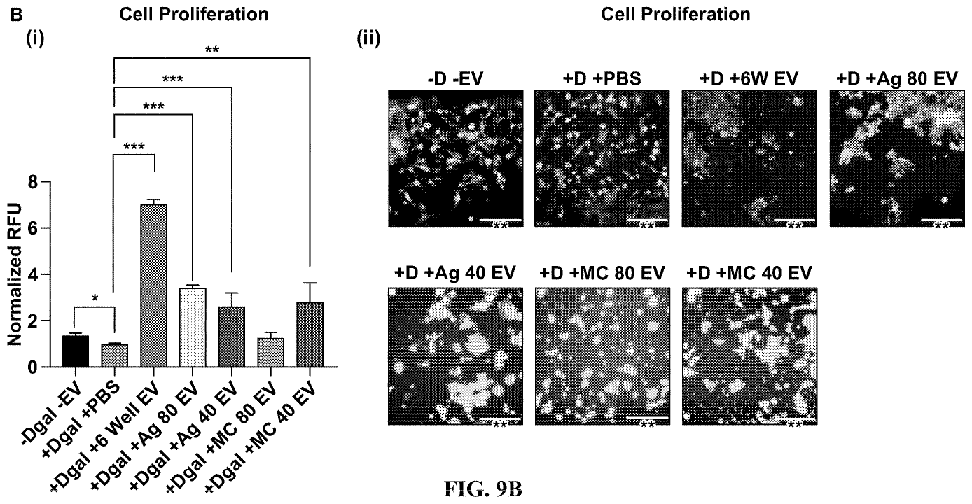

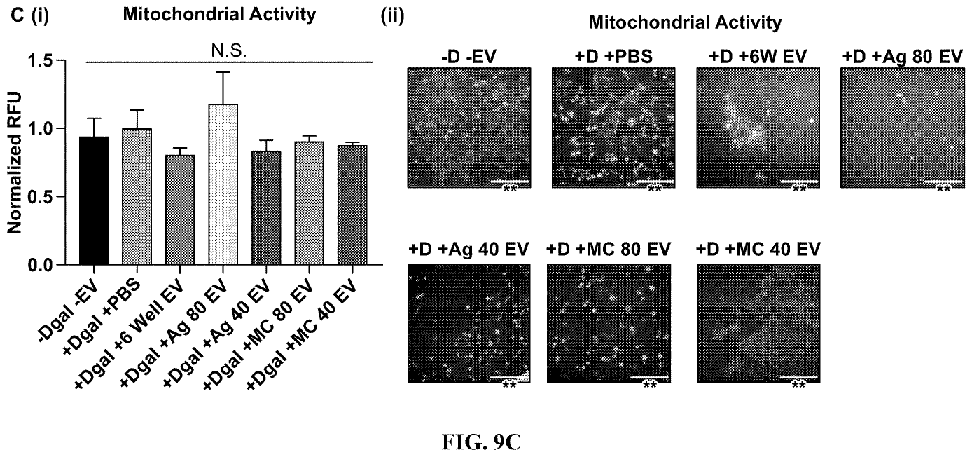

An induced in vitro senescence model was used for evaluating EV therapeutic potentials. Human blood vessel organoids (iBVO) derived from iPSCs were incubated for 48 hours with 150 mM of senescence inducing agent D-Galactose and then treated with EVs from different culture conditions at a dose of 2×10⁶ EVs/cell. ROS assay showed that D-galactose increased ROS when compared to no treatment, indicating successful senescence induction (FIG. 9A). The fluorescence was normalized to the average value of the cells in a senescent state. EV treatment from the MC 80 condition did not affect ROS production, but every other culture condition (6 well, Ag 80, Ag 40 and MC 40) significantly reduced ROS generation. For CellTrace proliferation assay, fluorescence was normalized to the inverse value of the average fluorescence of the cells in a senescent state (FIG. 9B). The results showed that D-galactose decreased cell proliferation when compared to no treatment, indicating successful senescence induction. EV treatment from the MC 80 condition provided no significant change in cell proliferation. Every other culture condition (6 well, Ag 80, Ag 40 and MC 40) significantly increased cell proliferation, indicating the therapeutic effect of the isolated iBVO EVs on senescence. The performance of MC 80 group may be due to the altered EV cargo or different optimal EV dose. Mitochondrial activity assay showed that D-galactose had no significant effect when compared to no treatment, indicating that D-galactose did not affect mitochondrial activity (FIG. 9C). EV treatment regardless of culture condition had no significant effect on mitochondrial activity.

### Example 8: VWBR Resulted in Different Metabolism and Gene Expression Compared to Static Culture

In the current application, human blood vessel organoids were derived from iPSCs in dynamic microenvironments utilizing VWBRs. These organoids were derived as aggregates, at a high shear rate (Ag 80) (corresponding to ~0.4 dyn/cm² on average) or low shear rate (Ag 40) (corresponding to ~0.2 dyn/cm² on average), as well as on microcarriers at a high shear rate (MC 80) or low shear rate (MC 40). The dynamic VWBR cultures were compared to a static aggregate culture (6-well). All five culture conditions had the same initial seeding density of 8×10⁴ cells/mL, but over time the VWBR conditions had similar or better cell growth than the static control, which can be shown via cell counting measurements as well as pH changes over the differentiation period. Metabolite concentrations, including glucose, lactate, and NH₄⁺ provide further evidence for increased cell growth of dynamic aggregates. The metabolite data of microcarrier culture are more similar to the dynamic aggregate cultures, which showed more aerobic metabolism (with the lactate to glucose ratio at 1.0-1.5) compared to the anaerobic metabolism (with the lactate to glucose ratio at around 2.0) in the static control. This similarity between dynamic aggregate and microcarrier cultures has been shown in the previous study. Chang et al. demonstrated that human forebrain spheroids derived from iPSCs in VWBRs as aggregates or on microcarriers shared more similarities on a metabolic level indicated by the lactate to glucose ratio, being more indicative of aerobic metabolism compared to the static control. It has been shown that VWBRs display increased stem cell expansion in a 3D dynamic environment when compared to static 2D cultures. In dynamic environments, the core of the aggregates suffered less from the poor nutrient, waste and oxygen transfer occurred in static cultures, which reduced cell viability near the center of aggregates. The static culture exhibited lower area and perimeter sizes than that of the Ag 40 condition. Regardless of shear rate however, the aggregate conditions displayed slightly lower cell-number-normalized glucose consumption.

Regarding differentiation, flow cytometry reveals that most culture conditions had no effect on differentiation efficiency, however the Ag 40 condition nearly doubled detection of key blood vessel markers such as CD31 and ZO-1. Most studies of iPSCs in VWBR focused on expansion rather than differentiation, and those that do primarily focused on aggregates. The utilization of microcarriers in VWBRs in the current application was shown not to impair differentiation potential, similar to high shear dynamic aggregate cultures and static control. At the molecular level, RT-qPCR analysis of cellular genes at D12 revealed similar or higher expression of glycolic pathway genes for Ag 40 and Ag 80 compared to the 6-well group, which was consistent with glucose metabolism determined by metabolite analysis. Consistent with the previous study of human MSC EV generation in VWBR, autophagy gene expression was upregulated in bioreactor culture compared to the static control, possibly due to the presence of shear stress. During EV biogenesis, multivesicular bodies (MVBs) can fuse with lysosomes where the contents are degraded in autophagy, or fuse with the plasma membrane where the intraluminal vesicles (ILVs) are secreted from cells as exosomes. For example, ATG5 can inhibit the vacuolar proton pump on MVBs/ILVs and prevent the content degradation, therefore redirecting MVB to the plasma membrane to be secreted as exosomes. Correlatively, ESCRT dependent and independent EV biogenesis genes were upregulated in VWBR compared to the static control. This correlation may indicate that the EV biogenesis is related to cellular metabolism and autophagy.

Consistent with EV biogenesis markers, the secreted EVs had higher yields (2-4 fold higher EVs per mL spent media) in VWBRs across three runs than the static control. When normalized to cell number, EV release was much higher (2-3 fold) in MC conditions than the aggregate conditions. The secreted EVs could be entrapped in the extracellular matrix network inside the aggregates, while the cells attached to the surface of MCs and formed tissue with lower thickness in the MC group that can easily release the EVs.

### Example: 9 Human Blood Vessel Organoid EV Cargo Analysis by Lipidomics

EVs from all the conditions had a mode size of ~200 nm, which means the dynamic environment did not significantly change the EV size. Both MC conditions showed the highest particle numbers per cell, 6-well condition showed the least yield, and the Ag conditions showed yields in the middle. When normalizing the EV number to the spent media volume, the VWBR conditions showed 2-3 fold higher EVs than the static control in the three runs. The pattern of EV secretion matched the expression of ESCRT independent and dependent EV biogenesis genes tested by RT-qPCR. This provides further evidence that VWBRs greatly improved EV production from iBVO. Microcarrier cultures generate a higher EV yield when normalized to cell number since the secreted EVs are readily released into the media, compared to EVs secreted from cells within organoids. This is consistent with the previous studies about iPSC-EVs from VWBRs as well as human forebrain spheroid-derived EVs from VWBRs, which had an increase in EV yield with microcarriers than aggregate cultures. In aggregate cultures, the cell-secreted EVs may be retained and sequestered by the extracellular matrix networks inside the organoids, with only the outer layer of the organoids exposed to shear stress. Shear stress has been proposed to cleave EVs once ligand binding creates membrane tethers. It has also been proposed that high intracellular levels of Ca²⁺ are related to EV production in MSCs. Consistently, the current application exhibited lower Ca²⁺ concentrations in the dynamic culture media than the static control, indicative of higher calcium flux and intracellular levels, thus promoting EV generation. Similar results were observed for K⁺ and Na⁺ concentrations, showing the higher ion flux and intracellular levels in dynamic culture than the static control.

The shear stress on the blood vessel organoids did not only have an effect on EV generation but may also affect the membrane composition and cargo packaging of the EVs. In the current application, EVs from 6-well, Ag 40, and MC 40 groups underwent lipidomic analysis and exhibited a distinct distribution in PCA plot. The Ag 40 condition had the most distinct population while the MC 40 and 6-well populations had large variations and some overlap, indicating that the lipid composition of those two conditions may be more similar to each other than the Ag 40 condition. The heatmap compared the distinct lipids tested in all samples and the upregulated or downregulated lipids were the least for MC 40 versus 6-well control. Regardless of the distinct lipid compositions of the tested samples, the total lipid molecule content remained similar between the samples and lipid compositions varied by less than 1%. Compared to the control, Ag 40 and MC 40 had lower concentration of unsaturated lipids per subclass, therefore having higher rigidity, and tighter packing of cargo at the sacrifice of membrane fluidity. Compared to the control, Ag 40 and MC 40 also had longer chain lengths of lipids per subclass also increasing EV rigidity. This rigidity could have resulted in harder EV uptake and less of a therapeutic effect when compared to the 6-well control. KEGG enrichment shows sphingolipid metabolism and signaling pathways are the most prevalent mechanisms that differ from the 6-well control. These lipids are key in membrane structure and cellular recognition and provide further evidence for the differences in EV permeability yielded by different microenvironments.

### Example 10: Function of Biomanufactured Human Blood Vessel Organoid-derived Evs

The therapeutic effect of human blood vessel organoid EVs was evaluated via an in vitro senescence model, in which 1×10⁴ cells/well of day 24 blood vessel organoids were incubated with 150 mM senescence-inducing D-Galactose for 48 hours and then treated with EVs from different conditions at a dose of 2×10⁶ EVs/cell for 24 hours. Common indicators of senescence include an increase in oxidative stress (i.e., ROS production), decrease in cellular proliferation, and decrease in mitochondrial activity. This may be due to ROS's ability to modulate miRNA families such as miR-34a whose downregulation and upregulation have been linked to neurological diseases. All these indicators can be observed in cells in a senescent state and in other neurological diseases such as Parkinson's disease and Alzheimer's disease.

In the current application, the increased ROS production was observed after 48 hr D-Galactose induction, indicative of cellular senescence. EVs from most culture conditions significantly decreased ROS to levels lower than prior to senescence induction (6-well, Ag 40, Ag 80, and MC 40). The significant therapeutic effect could be attributed to the high dosage of EVs used, ensuring high cellular uptake. MC 80 EVs had no therapeutic effect on ROS despite the high EV dosage, indicating the cell culture condition had an adverse effect on the EVs. It is unknown whether the high shear rate had an effect on ESCRT dependent and independent packing of miRNAs or other relevant cargo. Ag 80, Ag 40, and MC 40 groups all yielded significant reduction in ROS at similar levels to the static 6-well culture. This suggests that human blood vessel organoid-derived EVs grown on microcarriers are more susceptible to changes in cargo packaging as shear rate is increased. This reduction of ROS is consistent with previous studies utilizing EVs derived from both human MSCs and iPSCs.

A CFSE cell-proliferation assay also confirmed the senescent state of the cells by the reduction in cell proliferation after D-galactose induction. Similar to the ROS assay, MC 80 EVs had no therapeutic effect, suggesting that any cargo packaging discrepancies that affected ROS reduction had an effect on cellular proliferation as well. Ag 80, Ag 40, and MC 40 all yielded a significant increase in cell proliferation higher than that of BVOs prior to inducing senescence. This increase of cell proliferation due to EV treatment is consistent with previous studies.

A mitoSox Green mitochondrial activity assay yielded no significance of EV therapeutics. EVs can affect mitochondrial pathways, for example, Peruzzotti-Jametti et al. showed that neural stem cell-derived EVs transport functional cargo to restore mitochondrial function in target cells as shown by an enrichment of mitochondrial proteins in mtDNA-deficient L929 Rho cells. The results in the current application more likely provide evidence that D-galactose induction did not significantly affect the mitochondrial pathways as it did with ROS and cell proliferation. The exact mechanism of D-galactose induced senescence is not fully understood but common theories such as oxidative stress, inflammation and mitochondrial disfunction have all been proposed as mechanisms.

The current application demonstrated that VWBR conditions generated 2-3 fold higher EVs (per mL) of human blood vessel organoids than static control, possibly due to more aerobic metabolism and upregulation of EV biogenesis genes. Dynamic aggregate differentiation at low shear stress promoted marker expression of human blood vessel organoids. While microcarrier-based differentiation at low shear stress generated more EVs than the aggregates, the aggregate condition results in developmentally more mature population. EV lipidomics revealed that Ag 40 and MC 40 had lower concentration of unsaturated lipids per subclass, therefore having higher rigidity. Sphingolipid metabolism and signaling pathways are the most prevalent mechanisms in Ag 40 and MC 40 that differ from the 6-well control. Ag-EVs and MC-EVs showed therapeutic effects on in vitro senescence model. The current disclosure reports a systematic and comprehensive analysis of EV lipidomics of human blood vessel organoids differentiated on microcarriers and as aggregates in a dynamic bioreactor (VWBRs). The current application provides a scalable biomanufacturing platform with defined lipid compositions for human blood vessel organoid EV production for the applications in drug screening and cell-free therapy in clinical settings.

The integration of dynamic bioreactors with advanced lipidomic profiling in the current application paves the way for refining EV-based therapies tailored for precision medicine. The scalable nature of this platform opens the door to producing EVs at clinical-grade quality, potentially transforming the landscape of regenerative medicine and drug delivery systems. With further optimization, this approach could lead to groundbreaking advancements in treating complex diseases such as neurodegenerative disorders and vascular pathologies, marking a significant leap toward personalized, cell-free therapeutic solutions.

It will be apparent to those skilled in the art that various modifications and variations can be made in the present disclosure without departing from the scope or spirit of the invention. Other examples of the disclosure will be apparent to those skilled in the art from consideration of the specification and practice of the methods disclosed herein. It is intended that the specification and examples be considered as exemplary only, with a true scope and spirit of the invention being indicated by the following claims.

What is claimed is:

1. A method of producing extracellular vesicles, comprising:
culturing stem cells in a vertical-wheel bioreactor (VWBR) containing culture media to differentiate the stem cells into organoids, wherein cells within the organoids secrete extracellular vesicles (EVs) into the culture media;
collecting a culture media supernatant, wherein the culture media supernatant comprises EVs secreted by the cells within the organoids;
filtering the culture media supernatant to obtain the EVs, thereby obtaining filtered culture media supernatant;
isolating the EVs from the filtered culture media supernatant, thereby obtaining isolated EVs; and
concentrating the isolated EVs, wherein the EVs are produced in a greater amount per unit volume of culture medium than EVs produced from a corresponding static culture of organoids.

2. The method of claim 1, wherein the stem cells are human induced pluripotent stem cells.

3. The method of claim 1, wherein the organoids comprise human induced blood vessel organoids (iBVOs).

4. The method of claim 2, wherein the human induced pluripotent stem cells are differentiated into organoids on microcarriers in a vertical wheel bioreactor.

5. The method of claim 4, wherein the microcarriers comprise synthetic matrix-coated microcarriers.

6. The method of claim 4, wherein the microcarriers are present at a concentration of about 15 g/L to about 25 g/L.

7. The method of claim 2, wherein the human induced pluripotent stem cells are seeded into VWBR at about 5×10⁴ cells/mL to about 8×10⁴ cells/mL.

8. The method of claim 1, wherein the VWBR is operated at an agitation rate of about 40 rpm to about 80 rpm during differentiating stem cells.

9. The method of claim 1, wherein the stem cells are cultured in mTeSR medium for about 2 days and subsequently in DMEM/F12 medium comprising B27, Wnt activator, and fibroblast growth factor for about 10 days.

10. The method of claim 9, wherein the Wnt activator comprises CHIR99021.

11. The method of claim 9, wherein the fibroblast growth factor comprises FGF2.

12. The method of claim 3, wherein the iBVOs express one or more vascular markers selected from the group consisting of CD31, ZO-1, VE-cadherin, VEGF, and CD105.

13. The method of claim 1, wherein the culture media supernatant is collected after about 12 days.

14. The method of claim 1, wherein filtering the culture media supernatant comprises centrifuging at about 500 g for about 5 minutes.

15. The method of claim 1, wherein isolating the filtered EVs comprise ultracentrifugation.

16. The method of claim 1, wherein concentrating the isolated EVs comprises contacting the isolated EVs with polyethylene glycol (PEG) and NaCl to precipitate the EVs.

17. The method of claim 1, wherein the EVs have a mode particle size of about 150 nm to about 250 nm.

18. The method of claim 1, wherein the EVs express one or more markers selected from the group consisting of HSC70, TSG101, and CD81.

19. The method of claim 1, wherein the EVs are produced from about 2-fold to about 6-fold greater amount per mL of culture media than EVs produced from the corresponding static culture of organoids.

20. The method of claim 1, wherein the VWBR upregulates one or more EV biogenesis genes in the cells within the organoids relative to the corresponding static culture of organoids.

21. The method of claim 20, wherein the one or more EV biogenesis genes are selected from the group consisting of Rab7a, Rab27b, SMPD2, SMPD3, ALIX, HRS, STAM1, and STAM2.

22. The method of claim 1, wherein the VWBR upregulates one or more glycolysis-associated genes in the cells within the organoids relative to the corresponding static culture of organoids.

23. The method of claim 22, wherein the one or more glycolysis-associated genes are selected from the group consisting of HK2, PDK1, LDHA, and PKM2.

24. The method of claim 1, wherein the vertical wheel bioreactor upregulates one or more autophagy-associated genes in the cells within the organoids relative to the corresponding static culture of organoids.

25. The method of claim 24, wherein the one or more autophagy-associated genes are selected from the group consisting of AMPK, ATG5, ATG16L1, BECN1, LAMP1, and TFEB.

26. The method of claim 1, wherein the EVs comprise lipids associated with sphingolipid metabolism, wherein the lipids associated with sphingolipid metabolism comprise one or more ceramide lipids, sphingomyelin lipids, hexosylceramide lipids, or sulfated hexosyl ceramide lipids.

27. The method of claim 26, wherein the EVs comprise one or more ceramide lipids selected from ceramide-non-substituted lipids (Cer-NS) and ceramide-NDS lipids (Cer-NDS).

28. The method of claim 1, wherein the EVs comprise a lipid composition comprising at least 18 upregulated lipid species or at least 14 downregulated lipid species relative to EVs produced from a corresponding static culture of organoids.

29. The method of claim 28, wherein the at least 18 upregulated lipid species comprise at least

3. ceramide lipids and the at least 14 downregulated lipid species comprise at least 1 lysophosphatidylcholine lipid or a lysophosphatidylcholine ether lipid (LPC-O).

30. The method of claim 28, wherein the lipid composition further comprises at least 1 differentially expressed sphingomyelin lipid relative to EVs produced from the corresponding static culture of organoids.
